(19) United States
(12) Patent Application Publication
(10) Pub. No.: US 2025/0312486 A1
(43) Pub. Date: Oct. 9, 2025

(54) COMPOSITIONS FOR RESTORING MECP2 GENE FUNCTION AND METHODS OF USE THEREOF

(71) Applicants: City of Hope, Duarte, CA (US); The Translational Genomics Research Institute, Phoenix, AZ (US)

(72) Inventors: Saswati Chatterjee, Altadena, CA (US); Swati Bijlani, Pasadena, CA (US); Ka Ming Pang, Arcadia, CA (US); Sampathkumar Rangasamy, Phoenix, AZ (US); Vinodh Narayanan

(21) Appl. No.: 18/856,956

(22) PCT Filed: Apr. 18, 2023
(86) PCT No.: PCT/US2023/065912

§ 371 Date: Oct. 15, 2024

Related U.S. Application Data
(60) Provisional application No. 63/332,062, filed on Apr. 18, 2022.

(51) Int. Cl. A61K 48/00; A61K 38/17; C07K 14/005; C12N 7/00; C12N 9/22; C12N 15/11; C12N 15/86; C12N 15/90

(52) U.S. Cl. CPC A61K 48/005; A61K 38/1709; C07K 14/005; C12N 7/00; C12N 9/222; C12N 15/111; C12N 15/86; C12N 15/907; C12N 2310/20; C12N 2750/14121; C12N 2750/14122; C12N 2750/14143

(57) ABSTRACT

Provided herein, inter alia, are gene therapy compositions and methods that can efficiently and safely restore MeCP2 gene function in subjects affected by Rett Syndrome.

### BACKGROUND

Rett Syndrome (RTT) is a debilitating and challenging genetic neurodevelopmental disorder observed almost exclusively in females, and is caused by heterozygous, de novo mutations in the MECP2 gene, located on chromosome Xq28. These mutations lead to a deficiency of the wild type (WT) MeCP2 protein in all cells including neurons. Males born with the MECP2 mutations rarely survive due to the presence of a single X-chromosome. Females with Rett Syndrome display random X-chromosome inactivation and are mosaic for MeCP2 expression in all tissues. A progressive loss of motor skills and speech, seizures and intellectual disability is observed in children with Rett Syndrome.

MeCP2 is a nuclear protein that functions as a methylation reader and regulates expression of thousands of genes through chromatin compaction at methylated sites and interaction with transcriptional regulators. The protein contains a methyl binding domain which binds to DNA, and a transcriptional repressor domain, with the C-terminal portion containing the NCoR/SMRT interaction domain. MeCP2 is universally expressed, but the highest levels are observed in neurons. Over 300 distinct mutations in the MECP2 gene have been reported in patients with RTT, with almost all occurring within exons 3 and 4, and mapping to the MBD and C-terminus of TRD. Missense mutations account for approximately 70% of RTT cases. It is a direct result of the loss of MeCP2 function.

RTT is caused by mutations in the X-linked MECP2 gene, leading to deficiency of the wild type MeCP2 protein in neurons. However, overexpression of MeCP2 also results in a severe neurodevelopmental disorder, demonstrating the critical importance of MeCP2 gene dosage. Thus, MECP2 gene dosage poses a significant challenge to traditional gene therapy approaches. There are currently no available therapies for RTT.

Accordingly, there is a need in the art for improved gene therapy and genome editing compositions and methods that can efficiently and safely restore MECP2 gene function in subjects affected by Rett Syndrome.

### SUMMARY

Provided herein, inter alia, are adeno-associated virus (AAV) compositions that can restore MECP2 gene function in cells, and methods for using the same to treat diseases associated with impairment of MECP2 gene function.

In embodiments, the AAV compositions and methods disclosed herein allow for highly efficient correction of mutations in a MECP2 gene in vivo, without the need for cleavage of genomic DNA using an exogenous nuclease (e.g., a meganuclease, a zinc finger nuclease, a transcriptional activator-like nuclease (TALEN), or an RNA-guided nuclease such as a Cas9).

Accordingly, provided herein, inter alia, are nonpathogenic, replication-defective stem cell-derived adeno-associated virus (AAVHSC) compositions and methods that make use of the disclosed AAV compositions to carry out high-fidelity, precise, homologous recombination-based seamless genome editing to correct pathogenic mutations associated with Rett Syndrome. In embodiments, the disclosed editing methods preserve all regulatory elements associated with the MECP2 gene, allowing physiologic expression and overcoming a major obstacle in genetic medicine.

### BRIEF DESCRIPTION OF THE DRAWINGS

FIG. 1. Map of the MECP2 editing AAV vector plasmid. The AAV vector contains the editing construct flanked by AAV2 inverted repeats (ITR) sequences. The editing region contains 522 bp of intron 2, followed by exon 3, intron 3, exon 4 and 797 bp of 3′ untranslated (UTR) sequence from the MECP2 gene. Unique linker sequences of 28 bp and 33 bp were inserted in introns 2 and 3, respectively, to allow detection of corrected insertion. In addition, a promoterless expression cassette consisting of a T2A sequence, followed by the Reporter open reading frame (ORF) was inserted immediately downstream of exon 4.

FIG. 2. Map and structure of a single-stranded AAV-based MECP2 editing vector. The MECP2 editing construct is flanked by AAV2 inverted repeats (ITR) sequences. The editing region contains 522 bp of Intron 2, followed by Exon 3, intron 3, Exon 4 and 797 bp of 3′ untranslated (UTR) region from MECP2 gene. Unique linker sequences of 28 bp and 33 bp were inserted in introns 2 and 3, respectively, to allow detection correct insertion. In addition, a promoterless expression cassette consisting of a T2A sequence, followed by the Reporter open reading frame (ORF) was inserted immediately downstream of Exon 4.

FIG. 3. Representative MECP2 editing vector titers obtained upon packaging with AAVHSC15 or AAVHSC7 using Herpes Simplex Virus (HSV) helper virus.

FIGS. 4A-C. AAVHSC15 editing of the MECP2 gene in primary fibroblasts from a Rett Syndrome patient carrying a R282X mutation. The results of flow cytometric analysis of Venus expression in Rett Syndrome patient-derived primary fibroblasts are shown. Fibroblasts from a Rett Syndrome patient carrying a heterozygous MECP2 mutation of R282X (from Dr. Narayanan, Tgen), were transduced with the AAVHSC15 MECP2 editing vector at an MOI: 150,000, 300,000 and 450,000. After 48h, Venus expression was analyzed by flow cytometry. The Venus expression cassette is promoterless. Therefore, Venus expression indicated correct targeted insertion downstream from the MECP2 promoter. Also shown is the dose response showing that the proportion of genome editing observed in patient-derived cells increases with the multiplicity of infection of the AAV editing vector.

FIGS. 5A-D. AAVHSC editing of the MECP2 gene in primary fibroblasts from Rett Syndrome patients carrying different MECP2 mutations. The results of flow cytometric analysis of Venus expression in primary patient derived fibroblasts after transduction with the AAVHSC15 MECP2 editing vector at MOI: 250,000 are shown. The results were obtained with cells from patients carrying the following mutations in the MECP2: (A) MECP2 R282X (from Dr Narayanan, TGen), (B) MECP2 R306C (GM11270 from Coriell Institute), (C) MECP2 R106W (GM11273 from Coriell Institute), and (D) MECP2 T158M (GM17880 from Coriell Institute).

FIGS. 6A-D. AAVHSC editing of the MECP2 Gene in B-lymphoblasts (B-LCLs) from Rett Syndrome patients carrying different MECP2 mutations. The results of flow cytometric analysis of Venus expression in patient derived B-lymphoblasts after transduction with the AAVHSC15 MECP2 editing vector at MOI: 250,000 are shown. The results were obtained with cells from patients carrying the following mutations in the MECP2: (A) MECP2 R106W (GM11300 from Coriell Institute), (B) MECP2 S134C (GM17538 from Coriell Institute), (C) MECP2 S134C (GM17539 from Coriell Institute), and (D) MECP2 S134C (GM17540 from Coriell Institute). B, C & D represent cells from different members of a single family where each member carries the same MECP2 mutation. GM17539 are cells from the mother who is not clinically affected, despite carrying the mutation, likely due to a gene dosage effect. GM17538 are cells from the clinically affected son and GM17540 are cells from the clinically affected daughter.

FIGS. 7A-C. Comparison of editing efficiency by AAVHSC7 and AAVHSC15 MECP2 editing vectors. The results of flow cytometric analysis of editing efficiency in Rett patient-derived cells transduced with either AAVHSC7 or AAVHSC15 MECP2 editing vectors at MOI: 150,000 are shown. (A) MeCP2 R282X fibroblasts, (B) MeCP2 S134C (GM17538 from Coriell Institute) B-LCLs, (C) MECP2 R106W fibroblasts.

FIG. 8. Summary of editing frequencies observed in Rett patient-derived cells as estimated by reporter gene expression by flow cytometry. The specific mutations are noted for each cell line. Cells were transduced with either (A) AAVHSC15 (MOI: 250,000) (GM17538, GM17539 and GM17540 are cells obtained from the same family, where GM17539 cells were obtained from the clinically unaffected mother, GM17538 were from the affected son and GM17540 were from the affected daughter), or (B) AAVHSC15 (MOI: 150,000) or AAVHSC7 (MOI: 150,000). (C) Graph showing percent editing for R282X, R106W, and male hemizygous S134C with AAVHSC15 and AAVHSC7 MECP2 editing vectors.

FIG. 9. Maps of MECP2 editing vector, and relevant portions of the MECP2 gene in wild type (WT) and mutant alleles in GM11273. GM11273 was derived from a female with two X chromosomes encoding a wild type and a mutant R106W MECP2 allele which are depicted in the bottom panel and middle panel of the figure, respectively. Genomic single nucleotide polymorphisms (SNP) and vector-encoded linker sequences are highlighted. Also shown are two SNPs: i) a T/C in intron 2 and ii) a C/T in intron 3. The editing vector encodes a T in intron 2 and a C intron 3. The mutant and WT X chromosome have C in intron 2. The mutant X chromosome has the C in intron 3. The WT X chromosome has T in intron 3. Primers used for targeted integration (TI) analyses are shown in red.

FIG. 10. Summary of the editing patterns observed from AAVHSC15-edited GM11273 patient-derived cells. The PCR product obtained from the TI assay was cloned and few of the clones were sequenced. The sequence analysis showed heterogeneous editing patterns with different homologous recombination cross-over points. SNP and linker sequences were used as markers to identify regions of crossover. The cross-over positions identified are shown in FIG. 11 (A-E).

FIGS. 11A-11E. Potential cross-over positions between the editing vector and the X chromosome (either WT or mutant allele as specified). Also shown is the outcome if homologous recombination (HR)-related crossovers occurred at positions 1 at the 5′ end and at positions either 2 or 3 at the 3′ end. GM11273 (R106W) fibroblasts were transduced with the AAVHSC15 editing vector at MOI: 250,000. The genomic DNA was extracted from transduced cells and analyzed by targeted insertion (TI) with chromosome-specific primers MeCP2-USD-F2 or MeCP2-USD-F3 which anneal to X chromosome sequences external to the region spanned by the editing vector. The 226-linker2-R anneals within linker 2 sequence in intron 3. The resulting PCR product (1474 bp with MeCP2-USD-F2 and 1450 bp with MeCP2-USD-F3) was only observed in transduced cells. The amplicon was cloned and sequenced.

FIG. 12. Sequence alignment of wild-type MECP2 gene (Line 1), unedited mutant GM17538 cells (Line 2) and edited, corrected GM17538 cells (Line 3).

FIG. 13. Potential crossover positions between the editing vector and the X chromosome resulting in correction of the S134C mutation. GM17538 (S134C) B-LCLs were transduced with the AAVHSC7 MECP2 editing vector at MOI: 150,000. The genomic DNA was extracted from transduced cells and analyzed by TI with chromosome-specific primers MeCP2-DSD-R2 which anneal to X chromosome sequences external to the region spanned by the editing vector. The 226-linker2-F1 anneals within linker 2 sequence in intron 3. The resulting PCR product was only observed in transduced cells. The amplicon was cloned and sequenced.

FIG. 14. Potential MECP2 editing outcomes after AAVHSC HR editing.

FIG. 15. Schematic representation of preclinical human MECP2 editing vectors. Top panel. MECP2 editing vector containing 1. Wild-type DNA sequence of 100 to 2000 bp of intron 2 with a 28 bp artificial linker sequence 1, 2. 351 bp of wild type Exon 3, 3. 789 bp of intron 3 with a 33 bp artificial linker sequence 4. 1081 bp of wild type Exon 4. 5.T2A ribosome skipping sequence and a reporter open reading frame is inserted downstream of the exon 4 coding sequence. 6. 100 to 2000 bp sequence of 3′ UTR downstream to reporter open reading frame and act as right homology arm. Entire editing DNA sequence is flanked by AAV2 5′ and 3′ Inverted Terminal Repeats (ITRs). Middle panel. Editing vector containing codon altered Exon 3. Other elements are identical to that of the top panel except Exon 3 is codon altered whereas the DNA sequence is altered but maintaining the frequency of codon usage and identity of the encoded amino acid sequence. Bottom panel. Editing vector containing codon altered Exon 4. Other elements are identical to that of the top panel except exon 4 is codon altered whereas the DNA sequence is altered but maintaining the frequency of codon usage and identity of the encoded amino acid sequence.

FIG. 16. Schematic representation of therapeutic editing vectors for correction of MECP2 mutations in Exon 4 of the human MECP2 gene (top), and therapeutic editing vectors for treatment of MeCP2 mutation on Exon 3 of the human MECP2 gene (bottom). Top panel. Therapeutic editing vector containing codon altered Exon 4 for correction of MeCP2 mutations in Exon 4. The vector contains 1. 100 to 2000 bp of intron 2, 2. Wild-type Exon 3, 3. Wild-type intron 3 4. Codon-altered Exon 4 whereas the DNA sequence is altered but maintaining the frequency of codon usage and identity of the encoded amino acid sequence and 5. 100 to 2000 bp of 3′ UTR that acts as right homology arm. The entire intron 2, Exon 3 and intron 3 acts as left homology arm. Entire editing DNA sequence is flanked by AAV2 5′ and 3′ Inverted Terminal Repeats (ITRs). Bottom panel. Therapeutic editing vector containing codon altered Exon 3 for correction of MeCP2 mutations in Exon 3. The vector contains 1. 100 to 2000 bp of intron 2 that acts as left homology arm, 2. Codon-altered Exon 3 whereas the DNA sequence is altered but maintaining the frequency of codon usage and identity of the encoded amino acid sequence, 3. Wild-type intron 3, 4. Wild-type Exon 4 and 5. 100 to 2000 bp of 3′ UTR The entire intron 3, exon 4 and 3′ UTR acts as right homology arm. Entire editing DNA sequence is flanked by AAV2 5′ and 3′ Inverted Terminal Repeats (ITRs).

FIG. 17. Schematic representation of mouse MECP2 editing vectors with luciferase reporter. Top panel. Mouse MeCP2 editing vector that contains 1. Wild-type Exon 3, 2. Wild-type intron 3 with a inserted 33 bp linker sequence 3. Wild-type Exon 4, 4. T2 and luciferase reporter downstream of the coding sequence of Exon 4 and 5. 100 to 2000 bp of 3′ UTR that acts as right homology arm. Entire editing DNA sequence is flanked by AAV2 5′ and 3′ Inverted Terminal Repeats (ITRs). Bottom panel. Codon altered mouse MeCP2 editing vector that contains 1. Wild-type Exon 3, 2. Wild-type intron 3 with a inserted 33 bp linker sequence 3. Codon-altered Exon 4 whereas the DNA sequence is altered but maintaining the frequency of codon usage and identity of the encoded amino acid sequence, 4. T2 and luciferase reporter downstream of coding sequence of Exon 4 and 5. 100 to 2000 bp of 3′ UTR that acts as right homology arm. Exon 3 and intron 3 acts as left homology arm. Entire editing DNA sequence is flanked by AAV2 5′ and 3′ Inverted Terminal Repeats (ITRs).

FIG. 18. Schematic representation of mouse MECP2 editing vectors for functional rescue. Top: wild type; middle: codon-altered Exon 3; Bottom: codon-altered Exon 4. Top panel. Mouse editing vector containing wild type Exon 3 and Exon 4 sequence. The vector also contains 100-2000 bp of Intron 2 and 100-2000 bp of 3′ ITR sequence. Entire editing DNA sequence is flanked by AAV2 5′ and 3′ Inverted Terminal Repeats (ITRs). Middle panel. Mouse editing vector containing codon-altered Exon 3. This vector is identical to that of the top panel except exon 3 is codon-altered. Bottom panel. Mouse editing vector containing codon-altered Exon 4. This vector is identical to that of the top panel except exon 4 is codon-altered.

FIG. 19A. Mapping of SEQ ID Nos. 1, 2, 3, 4, 5, 6, 8, 10, 11 and 12 on the MECP2 editing vector maps. FIG. 19B. Mapping of SEQ ID Nos. 7, 8, 9 and 10 on the MECP2 editing vector maps.

FIG. 20. Derivation of first generation MECP2 editing AAV vectors. VSC224 was obtained by cloning Linker 2 into Intron 3 of VSC131 to allow identification of edited genomes. VSC225 was obtained by cloning Linker 1 into VSC224. VSC226 was obtained by cloning the T2A-Venus-pA cassette into VSC225.

FIG. 21. Derivation of second generation MECP2 editing AAV vectors. VSC419 was obtained from VSC226. It encodes a codon-altered Exon 3 and a wild type Exon 4. The VSC419 vector forces the replacement of the genomic Exon 3 with the codon-altered Exon 3, which corrects all mutations in Exon 3. The VSC420 vector was obtained from VSC226. It encodes a wild type Exon 3 and a codon altered Exon 4. The VSC420 vector forces the replacement of the genomic Exon 4 with the codon-altered Exon 4, which corrects all mutations in Exon 4.

FIG. 22. Third generation MECP2 editing AAV vector VSC433, which combines the codon-altered sequence of Exon 3 and Exon 4 and forces correction of all mutations in Exons 3 and 4, which comprise ˜90% of MECP2 mutations associated with Rett Syndrome.

FIG. 23. Maps of (A) murine codon-altered Exon 4 MECP2 editing AAV vector VSC418; (B) murine MECP2 editing AAV vector VSC414.

FIG. 24. Maps of the editing vector and mutant MECP2 alleles in male GM17538 cells. GM17538 was derived from a male with the X chromosomes encoding a mutant MECP2 allele. Genomic single nucleotide polymorphisms (SNP) and vector-encoded linker sequences are highlighted. Also shown is a SNPs: a T/C in intron 2. The editing vector encodes a T in intron 2. The mutant X chromosome has a C in intron 2. Primers used for 5′ targeted insertion (TI) analyses are also shown.

FIG. 25. Editing Patterns Observed from 5′ Sequence Analysis of AAVHSC7-Edited GM17538 RTT Patient-derived Cells. The PCR product obtained from the 5′ TI assay was cloned and few of the clones were sequenced. The sequencing analysis of clones resulted in the identification of different editing patterns dependent on the point of cross-over between the vector and the genomic sequence, which was identified using the SNPs and linker sequences as markers. The sequencing indicated that the 5′ junction sequence in edited cells were intact. The respective sequences of clones are shown in text.

FIG. 26. Maps of the Editing Vector and Mutant and Wild Type MECP2 Alleles in R282X cells. R282X cells were derived from a female with two X chromosomes encoding a wild type and a mutant MECP2 allele. Genomic single nucleotide polymorphisms (SNP) and vector-encoded linker sequences are highlighted. Also shown are two SNPs: i) a T/C in intron 2, ii) a C/T in intron 3 and iii) a C/T in exon 4 resulting in a silent mutation. The editing vector encodes a T in intron 2 and a C intron 3. The mutant and WT X chromosome have C in intron 2 and T in intron 3. Primers used for 5′ and 3′ TI analyses are also shown.

FIG. 27. Editing Patterns Observed from 5′ Sequence Analysis of AAVHSC7-Edited R282X Patient-derived Cells. Summary of the editing patterns observed from AAVHSC7-edited R282X patient-derived cells. The PCR product obtained from the 5′ TI assay was cloned and few of the clones were sequenced. The sequencing analysis of clones resulted in the identification of different editing patterns dependent on the point of cross-over between the vector and the genomic sequence, which was identified using the SNPs and linker sequences as markers. The sequencing indicated that the 5′ junction sequence in edited cells were intact. The respective sequences of clones are shown in text.

FIG. 28. Editing Patterns Observed from 3′ Sequence Analysis of AAVHSC7-Edited R282X Patient-derived Cells. Summary of the editing patterns observed from AAVHSC7-edited R282X patient-derived cells. The PCR product obtained from the 3′ TI assay was cloned and few of the clones were sequenced. The sequencing analysis of clones resulted in the identification of different editing patterns dependent on the point of cross-over between the vector and the genomic sequence, which was identified using the SNPs and linker sequences as markers. The respective sequences of clones are shown in text. 7 out of 9 (78%) clones (Pattern A) had the correct amino acid at position 282.

FIG. 29. Graph showing the comparative editing efficiency between the original MECP2 editing vector VSC226 and the codon-altered (CA) Exon 4 MECP2 editing vector VSC420. The results of flow cytometric analysis of editing efficiency in Rett Syndrome patient-derived cells transduced with either vector (packaged with AAVHSC7) at MOI: 150,000 are shown. (A) MeCP2 R282X fibroblasts (n=4, 3), (B) MeCP2 r.378_384delTCCCCAG fibroblasts (GM21921; n=3, 2), and (C) MeCP2 R133C B-LCLs (GM23659; n=4, 4).

FIG. 30. MeCP2 expression using immunostaining in different cells: (A) Wild-type fibroblasts AG21802 containing wild-type MeCP2 protein; (B) MeCP2 r.378_384delTCCCCAG male-patient derived fibroblasts (GM21921) containing a deletion in the beginning of Exon 4 resulting in frame-shift mutation and stop codon. Cells are from a male donor who was hemizygous for a microdeletion in exon 4 of the MECP2 gene r.378_384delTCCCCAG, resulting in a frameshift that leads to a premature stop codon; (A) The wild-type fibroblasts AG21802 expressed MeCP2 that was localized to the nucleus. (B) The untransduced GM21921 cells did not express MeCP2 due to a deletion in C-Ter resulting in truncation of protein, which is not recognized by the C-Ter epitope binding antibody.

FIG. 31. MeCP2 expression using immunostaining in RTT patient-derived GM21921 cells 7 days after transduction with AAVHSC7-vSC226. The cells are from a male donor who was hemizygous for a microdeletion in exon 4 of the MECP2 gene r.378_384delTCCCCAG, resulting in a frameshift that leads to a premature stop codon. The cells were transduced with an AAVHSC7 editing vector at MOI:150,000. After editing approximately 10% of the cells show expression of MECP2 in the nuclei, representing edited cells.

FIG. 32. MeCP2 expression using immunostaining in RTT patient-derived GM21921 cells 7 days after transduction with AAVHSC7-vSC226. The cells are from a male donor who was hemizygous for a microdeletion in exon 4 of the MECP2 gene r.378_384delTCCCCAG, resulting in a frameshift that leads to a premature stop codon. The cells were transduced with an AAVHSC7 editing vector at MOI:150,000. After editing approximately 10% of the cells show expression of MECP2 in the nuclei, representing edited cells.

FIG. 33. MeCP2 expression using immunostaining in RTT patient-derived GM21921 cells 7 days after transduction with AAVHSC7-vSC226. The cells are from a male donor who was hemizygous for a microdeletion in exon 4 of the MECP2 gene r.378_384delTCCCCAG, resulting in a frameshift that leads to a premature stop codon. The cells were transduced with an AAVHSC7 editing vector at MOI:150,000. After editing approximately 10% of the cells show expression of MECP2 in the nuclei, representing edited cells.

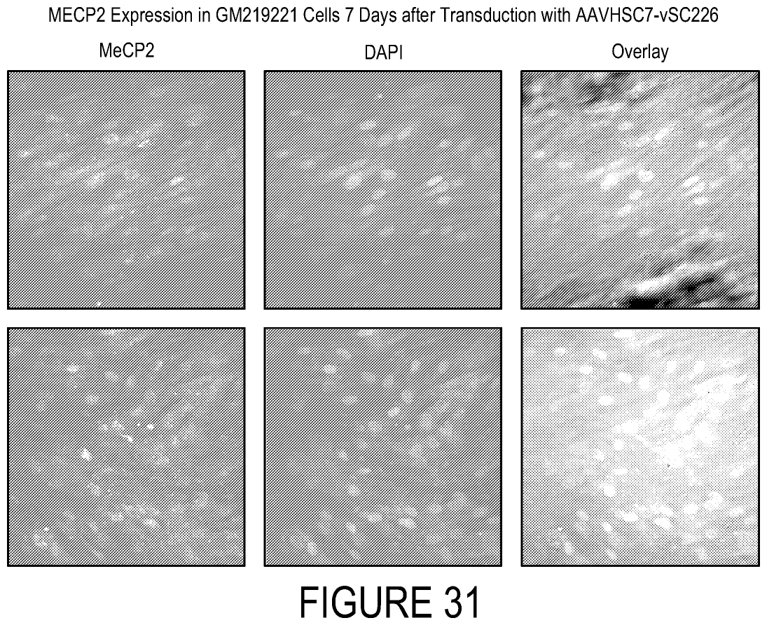

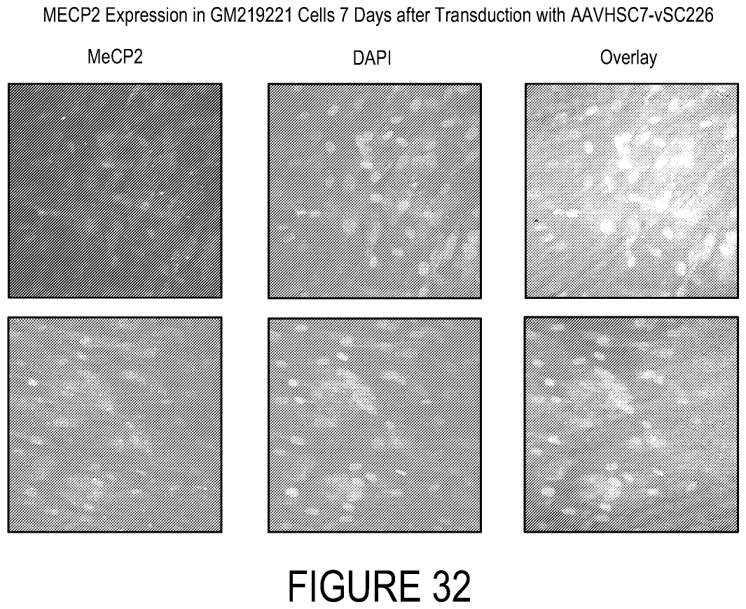

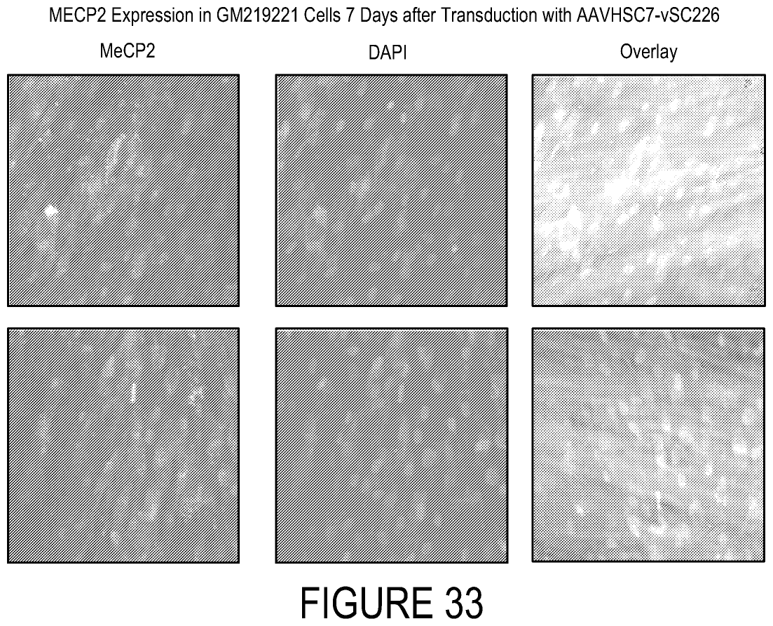

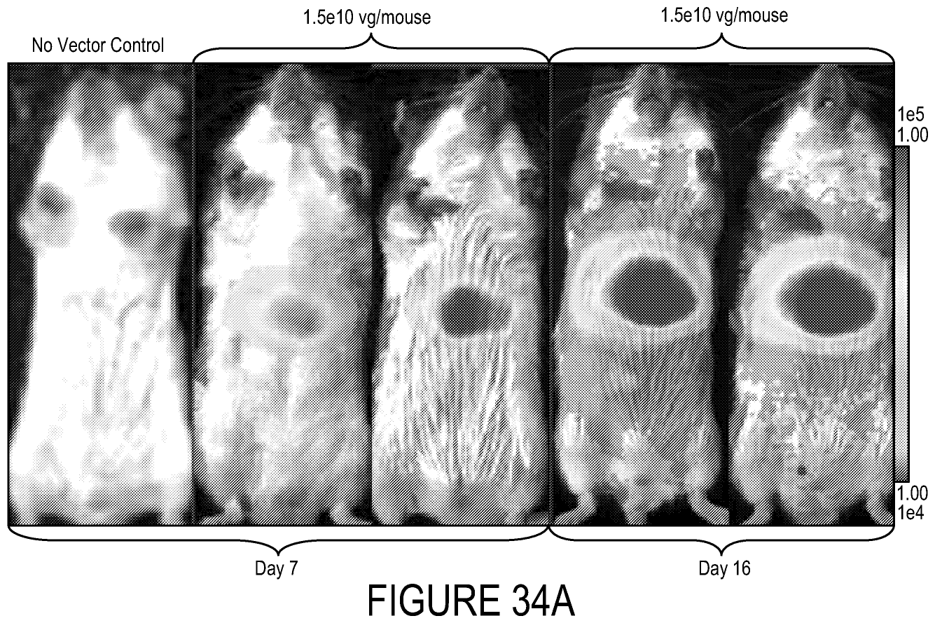

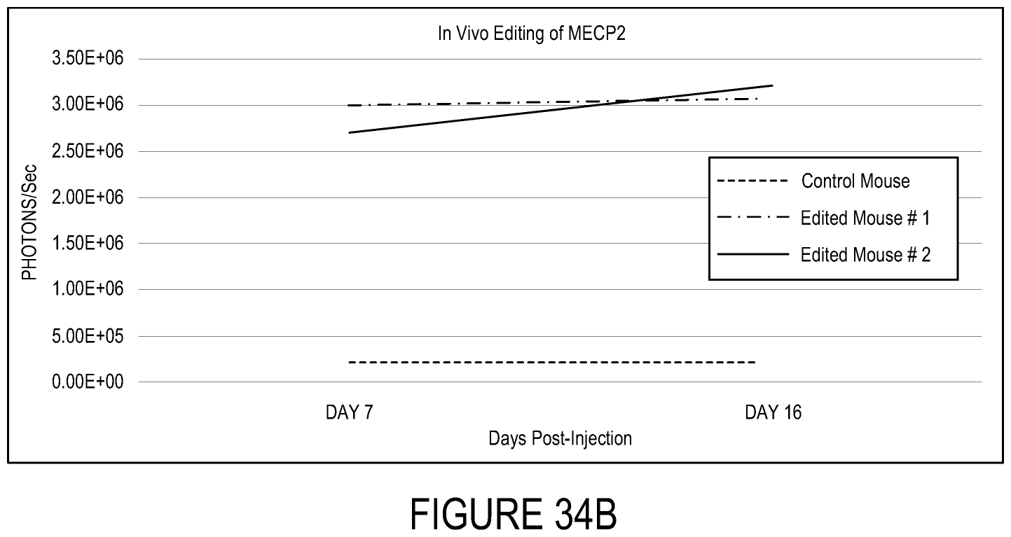

FIGS. 31-33 illustrate different fields within the RTT patient-derived GM21921 cells, showing that the restoration of MECP2 expression is widespread and not limited to a small number of cells.

FIG. 34. In vivo editing of the murine MECP2 gene by the codon-altered murine MeCP2 editing vector AAVHSC15 VSC418. The editing vector inserted a promoterless luciferase open reading frame immediately downstream of the MECP2 Exon 4. A. Serial in vivo bioluminescent imaging shows luciferase expression in mice injected intravenously with the codon-altered Exon 4 mouse MeCP2 editing vector packaged in AAVHSC15. Two mice were injected with the editing vector at 1.5e+10 vg/mouse. Also shown is a negative control uninjected mouse. The mice were injected intraperitoneally with the luciferin substrate and imaged on days 7 and 16 post vector administration to measure the flux from luciferase expression. B. Kinetics of luciferase expression over time after injection of the AAVHSC editing vector. Luciferase expression (photons per second) is plotted over time after injection.

### DETAILED DESCRIPTION

The instant disclosure provided adeno-associated virus (AAV) compositions and methods that can restore MECP2 gene function in a cell.

### Definitions

As used herein, the term “replication-defective adeno-associated virus” refers to an adeno-associated virus (AAV) that requires the presence of a helper virus, such as an adenovirus or a herpes virus. In embodiments, the replication-defective adeno-associated virus is defective for one or more functions that are essential for viral genome replication or synthesis and assembly of viral particles within a cell. In embodiments, the replication-defective adeno-associated virus has a decreased replicative capacity relative to an adeno-associated virus that replicated normally. In embodiments, the AAV comprises a genome lacking replication (Rep) genes, capside (Cap) genes, or both Rep genes and Cap genes. In embodiments, the AAV comprises a genome lacking Rep genes. In embodiments, the AAV comprises a genome lacking Cap genes. In embodiments, the AAV comprises a genome lacking Rep genes and Cap genes.

As used herein, the term “Rep gene” refers to a gene, which through the use of two promoters and alternative splicing, encodes four regulatory proteins involved in AAV genome replication. In embodiments, the four regulatory proteins include Rep78, Rep68, Rep52 and Rep40.

As used herein, the term “Cap gene” refers to a gene which encodes three capsid proteins. In embodiments, the three capsid proteins include virion protein 1 (VP1), virion protein 2 (VP2), and virion protein 3 (VP3). In embodiments, all VPs share a common C-terminal VP3 sequence. In embodiments, the VP2 N-terminal region is the VP1/VP2 common region. In embodiments, the viral proteins VP1, VP2, and VP3 assemble to form the T=1 icosahedral capsid consisting of 60 viral proteins.

As used herein, the term “MECP2 gene” refers to a wild-type or mutant gene that encodes the protein MeCP2. In embodiments, the MeCP2 protein activates and represses transcription. In embodiments, MeCP2 binds methylated CpGs. In embodiments, MeCP2 is a chromatin-associated protein. In embodiments, the MECP2 gene is located on the long (q) arm of the X chromosome in band 28 (“Xq28”), from base pair 154,021,573 to base pair 154,097,717. In embodiments, MeCP2 is X-linked and subject to X inactivation. In embodiments, genetic mutations in the coding region of the X-chromosome-linked MECP2 gene cause Rett syndrome.

As used herein, the term “correcting a mutation in a MECP2 gene” refers to the insertion, deletion, or substitution of one or more nucleotides at a target locus in a mutant MECP2 gene to create a MECP2 gene that is capable of expressing a wild-type MeCP2 polypeptide or a functional equivalent thereof. In certain embodiments, “correcting a mutation in a MECP2 gene” involves inserting a nucleotide sequence encoding at least a portion of a wild-type MeCP2 polypeptide or a functional equivalent thereof into the mutant MECP2 gene. A skilled person in the art will appreciate that the portion of a correction genome comprising the 5′ homology arm, editing element, and 3′ homology arm can be in the sense or antisense orientation relative to the target locus.

As used herein, the term “correction genome” refers to a recombinant AAV genome that is capable of inserting an editing element (e.g., one or more nucleotides or an internucleotide bond) via homologous recombination into a target locus to correct a genetic defect in a MECP2 gene. In certain embodiments, the target locus is in the human MECP2 gene. The skilled artisan will appreciate that the portion of a correction genome comprising the 5′ homology arm, editing element, and 3′ homology arm can be in the sense or antisense orientation relative to the target locus.

As used herein, the term “editing element” refers to the portion of a correction genome that when inserted at a target locus modifies the target locus. An editing element can mediate insertion, deletion, or substitution of one or more nucleotides at the target locus.

As used herein, the term “target locus” refers to a region of a chromosome or an internucleotide bond (e.g., a region or an internucleotide bond of the human MECP2 gene) that is modified by an editing element.

As used herein, the term “homology arms” or “homology arm” refers to genomic DNA fragments flanking a gene. In embodiments, the homology arms comprise two genomic DNA fragments, one at the 5′ end of the gene (5′ homology arm), and one at the 3′ end of the gene (3′ homology arm). In embodiments, each homology arm comprises a portion of a correction genome positioned 5′ or 3′ to an editing element that is substantially identical to the genome flanking a target locus. In embodiments, the target locus is in a human MECP2 gene, and the homology arm comprises a sequence substantially identical to the genome flanking the target locus.

As used herein, the term “mutation-deficient MECP2 nucleic acid sequence” refers to a sequence that encodes a wild-type MeCP2 protein. In embodiments, a mutation-deficient MECP2 nucleic acid sequence includes the wild-type MECP2 nucleotide sequence. In embodiments, a mutation-deficient MECP2 nucleic acid sequence includes a codon-altered MECP2 nucleic acid sequence that differs from the wild type nucleotide sequence by including a different single base within a specific codon and encodes the wild-type MeCP2 protein sequence. In embodiments, a mutation-deficient MECP2 nucleic acid sequence includes a codon-optimized MECP2 nucleic acid sequence that differs from the wild type nucleotide sequence by including a different single base within a specific codon and encodes the wild-type MeCP2 protein sequence. In embodiments, a “mutation-deficient” MECP2 nucleic acid sequence consists of any nucleotide sequences that encode the wild-type MeCP2 protein. In embodiments, a “mutation-deficient” MECP2 nucleic acid sequence comprises one or more different single bases within specific codons that force recombination outside the exon area while maintaining the correct protein sequence and codon usage. In embodiments, a “mutation-deficient” MECP2 nucleic acid sequence maintains the proper physiological level of functional MECP2 proteins.

As used herein, the term “capsid” refers to the protein shell of a virus. In embodiments, the capsid encloses the genetic material of the virus.

As used herein, the term “Clade F capsid protein” refers to an AAV VP1, VP2, or VP3 capsid protein.

As used herein, the term “a disease or disorder associated with a MECP2 gene mutation” refers to any disease or disorder caused by, exacerbated by, or genetically linked with mutation of a MECP2 gene. In certain embodiments, the disease or disorder associated with a MECP2 gene mutation is Rett Syndrome.

As used herein, the term “coding sequence” refers to the portion of a nucleic acid sequence, such as a complementary DNA (cDNA), which encodes a polypeptide, starting at the start codon and ending at the stop codon. A gene may have one or more coding sequences due to alternative splicing and/or alternative translation initiation. A coding sequence may either be wild-type or silently altered.

As used herein, the term “silently altered” or “silent alteration” refers to alteration of a coding sequence of a gene (e.g., by nucleotide substitution) without changing the amino acid sequence of the polypeptide encoded by the gene. In embodiments, silent alteration does not change the expression level of a coding sequence. In embodiments, silent alteration increases on-targeting editing events.

As used herein, the term “exon” refers to a portion of a gene that encodes a protein. In embodiments, exons are mRNA coding regions that code for amino acids. In embodiments, the MECP2 gene comprises four exons.

As used herein, the term “intron” refers to a portion of a gene that does not encode a protein. In embodiments, introns do not remain in the mature mRNA molecule following transcription of the gene. In embodiments, the MECP2 gene comprises three introns. In embodiments, the four exons and three introns in the MECP2 gene are alternatively spliced to generate two protein isoforms MECP2-E1 and MECP2-E2.

In the instant disclosure, exons and introns in a MECP2 gene are specified relative to the exon encompassing the first nucleotide of the start codon. The exon encompassing the first nucleotide of the start codon is exon 1. Exons 3′ to exon 1 are from 5′ to 3′: exon 2, exon 3, etc. Introns 3′ to exon 1 are from 5′ to 3′: intron 1, intron 2, etc. Accordingly, the MECP2 gene comprises from 5′ to 3′: exon 1, intron 1, exon 2, intron 2, exon 3, etc. A skilled person will appreciate that a gene may be transcribed into multiple different mRNAs. As such, a gene (e.g., MECP2) may have multiple different sets of exons and introns. As used herein, the term “insertion” refers to introduction of an editing element into a target locus of a target gene by homologous recombination between a correction genome and the target gene. Insertion of an editing element can result in substitution, insertion and/or deletion of one or more nucleotides in a target gene. For example, in certain embodiments, the term “insertion” refers to introduction of an editing element into a target locus of a MECP2 gene by homologous recombination between a correction genome and the MECP2 gene. Insertion of an editing element can result in substitution, insertion and/or deletion of one or more nucleotides in a MECP2 gene.

As used herein, the term “insertion efficiency of the editing element into the target locus” refers to the percentage of cells in a transduced population in which insertion of the editing element into the target locus has occurred.

As used herein, the term “allelic frequency of insertion of the editing element into the target locus” refers to the percentage of alleles in a population of transduced cells in which insertion of the editing element into the target locus has occurred.

As used herein, the term “standard AAV transduction conditions” refers to transduction of cells with an AAV at a multiplicity of infection (MOI) of 1.5×10⁵, wherein the cells are cultured in DMEM media supplemented with GlutaMAX and 10% heat inactivated (HI)-fetal calf serum (FCS), and 2 mmol/L L-glutamine at 37° C. in an incubator environment of 5% carbon dioxide (CO₂), wherein the cells in log phase growth are seeded at approximately 200,000 cells per ml and infected on the same day for B-LCLs or the next day for fibroblasts, wherein the AAV is formulated in phosphate buffered saline (PBS), and wherein the AAV is added to the cell culture medium containing the B lymphoblastoid cells in a volume that is less than or equal to 5% of the volume of the culture medium.

As used herein, the term “Rett Syndrome” refers to a rare neurological disorder that occurs almost exclusively in girls and leads to several impairments. In embodiments, Rett Syndrome is caused by mutations on the X chromosome on the MECP2 gene. In embodiments, Rett Syndrome includes loss of speech, loss of purposeful use of hands, involuntary hand movements, loss of mobility or gait disturbances, loss of muscle tone, seizures, scoliosis, breathing issues, sleep disturbances, and slowed rate of growth for head, feet and hands.

As used herein, the term “effective amount” in the context of the administration of an AAV to a subject refers to the amount of the AAV that achieves a desired prophylactic or therapeutic effect.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Nucleic acid” refers to deoxyribonucleotides or ribonucleotides and polymers thereof in either single- or double-stranded forms, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

The term “nucleotide” typically refers to a compound containing a nucleoside or a nucleoside analogue and at least one phosphate group or a modified phosphate group linked to it by a covalent bond. Exemplary covalent bonds include, without limitation, an ester bond between the 3′, 2′ or 5′ hydroxyl group of a nucleoside and a phosphate group.

The term “nucleoside” refers to a compound containing a sugar part and a nucleobase, e.g., a pyrimidine or purine base. Exemplary sugars include, without limitation, ribose, 2-deoxyribose, arabinose and the like. Exemplary nucleobases include, without limitation, thymine, uracil, cytosine, adenine, guanine.

The term “nucleoside analogue” may refer to a nucleoside any part of which is replaced by a chemical group of any nature. Exemplary nucleoside analogues include, without limitation, 2′-substituted nucleosides such as 2′-fluoro, 2-deoxy, 2′-O-methyl, 2′-O—P-methoxyethyl, 2′-O-allylriboribonucleosides, 2′-amino, locked nucleic acid (LNA) monomers and the like. The term “nucleoside analogue” may also refer to a nucleoside in which the sugar or base part is modified, e.g. with a non-naturally occurring modification. Exemplary nucleoside analogues in which the sugar part is replaced with another cyclic structure include, without limitation, monomeric units of morpholinos (PMO) and tricyclo-DNA. Exemplary nucleoside analogues in which the sugar part is replaced with an acyclic structure include, without limitation, monomeric units of peptide nucleic acids (PNA) and glycerol nucleic acids (GNA). Suitably, nucleoside analogues may include nucleoside analogues in which the sugar part is replaced by a morpholine ring.

Nucleoside analogues may include deoxyadenosine analogues, adenosine analogues, deoxycytidine analogues, cytidine analogues, deoxyguanosine analogues, guanosine analogues, thymidine analogues, 5-methyluridine analogues, deoxyuridine analogues, or uridine analogues. Examples of deoxyadenosine analogues include didanosine (2′, 3′-dideoxyinosine) and vidarabine (9-0-D-arabinofuranosyladenine), fludarabine, pentostatin, cladribine. Examples of adenosine analogues include DCX4430 (Immucillin-A). Examples of cytidine analogues include gemcitabine, 5-aza-2′-deoxycytidine, cytarabine. Examples of deoxycytidine analogues include cytarabine, emtricitabine, lamivudine, zalcitabine. Examples of guanosine and deoxyguanosine analogues include abacavir, acyclovir, entecavir. Examples of thymidine and 5-methyluridine analogues include stavudine, telbivudine, zidovudine. Examples of deoxyuridine analogues include idoxuridine and trifluridine.

The terms “purine analogue” or “pyrimidine analogue” refers to modifications, optionally non-naturally occurring modifications, in the nucleobase, for example hypoxanthine, xanthine, 2-aminopurine, 2,6-diaminopurine, 6-azauracil, 5-methylcytosine, 4-fluorouracil, 5-fluoruracil, 5-chlorouracil, 5-bromouracil, 5-iodouracil, 5-trifluoromethyluracil, 5-fluorocytosine, 5-chlorocytosine, 5-bromocytosine, 5-iodocytosine, 5-propynyluracil, 5-propynylcytosine, 7-deazaadenine, 7-deazaguanine, 7-deaza-8-azaadenine, 7-deaza-8-azaguanine, isocytosine, isoguanine, mercaptopurine, thioguanine. Exemplary pyrimidine analogues include, without limitation, 5-position substituted pyrimidines, e.g. substitution with 5-halo, 5′-fluoro. Examples of purine analogues include, without limitation, 6- or 8-position substituted purines, e.g., substitution with 5-halo, 5′-fluoro.

The term “phosphate group” as used herein refers to phosphoric acid H₃PO₄ wherein any hydrogen atoms are replaced by one, two or three organic radicals to give a phosphoester, phosphodiester, or phosphotriester, respectively. Oligonucleotides may be linked by phosphodiester, phosphorothioate or phosphorodithioate linkages.

In structures of this type, it will be appreciated that the labels 3′ and 5′, as applied to conventional sugar chemistry, apply by analogy.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA or mRNA produced by the cell. The level of expression of non-coding nucleic acid molecules may be detected by standard PCR or Northern blot methods well known in the art. See, Sambrook et al., 1989 Molecular Cloning: A Laboratory Manual, 18.1-18.88.

The terms “transfection”, “transduction”, “transfecting” or “transducing” are used interchangeably throughout and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, 7-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the glutamic acid 138 residue.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified (e.g., labeled).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then considered to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Any methods of alignment of sequences for comparison well known in the art are contemplated. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

Example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically or substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Point Mutation” or “Codon-Altered Mutation” as referred herein means substitution of a single base in a nucleic acid. The base substitution can be a silent mutation where the altered codon corresponds to the same amino acid, or a missense mutation where the altered codon corresponds to a different amino acid, or a nonsense mutation where the altered codon corresponds to a stop signal. In embodiments, a codon-altered mutation is one mutated base within a single codon.

“Codon-altered sequences” as provided herein are base substitutions or silent mutations, where the altered codon corresponds to the same amino acid in the MeCP2 protein. As provided herein, base substitutions in a nucleotide sequence are in lower case letters.

As disclosed herein, codon alteration ensures retention of endogenous regulatory sequences and occurrence of precise cross-over and maintains correct level of MeCP2 protein expression.

The term “sample” includes sections of tissues such as biopsy and autopsy samples, and frozen sections taken for histological purposes. Such samples include blood and blood fractions or products (e.g., bone marrow, serum, plasma, platelets, red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms; fully or partially remove the disease's underlying cause; shorten a disease's duration; or do a combination of these things.

“Treating” and “treatment” as used herein also include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” is used in accordance with its plain and ordinary meaning and includes oral administration, administration by inhalation, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Selective” or “selectivity” or the like of a compound refers to the compound's ability to discriminate between molecular targets.

“Specific”, “specifically”, “specificity”, or the like of a compound refers to the compound's ability to cause a particular action, such as inhibition, to a particular molecular target with minimal or no action to other proteins in the cell.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

As defined herein, the term “inhibition”, “inhibit”, “inhibiting” and the like in reference to a disease or disorder means reduction of a disease or disorder or reduction of symptoms of a disease or disorder. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The term “expression” includes any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “associated” or “associated with” in the context of a substance or substance activity or function associated with a disease (e.g., a protein associated with an infectious disease) means that the disease (e.g., cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease. For example, a symptom associated with a disease, such as COVID-19 may be treated with an S protein modulator or S protein inhibitor, in the instance where S protein activity or function causes the disease (e.g., COVID-19).

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

“Pharmaceutically acceptable excipient” and “pharmaceutically acceptable carrier” refer to a substance that aids the administration of an active agent to and absorption by a subject and can be included in the compositions described herein without causing a significant adverse toxicological effects on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylase or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions described herein. One of skill in the art will recognize that additional pharmaceutical excipients may be useful. The term “pharmaceutically acceptable salt” refers to salts derived from a variety of organic and inorganic counter ions well known in the art and include, by way of example only, sodium, potassium, calcium, magnesium, ammonium, tetraalkylammonium, and the like.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Adeno-Associated Virus Compositions

In embodiments, provided herein are novel replication-defective AAV compositions useful for restoring correct MECP2 expression in cells with reduced or otherwise defective MECP2 gene function. In embodiments, the AAV compositions are highly efficient at correcting mutations in the MECP2 gene or restoring MECP2 expression, and do not require cleavage of the genome at the target locus by the action of an exogenous nuclease (e.g., a meganuclease, a zinc finger nuclease, a transcriptional activator-like nuclease (TALEN), or an RNA-guided nuclease such as a Cas9) to facilitate such correction. Accordingly, in embodiments, the AAV composition disclosed herein does not comprise an exogenous nuclease or a nucleotide sequence that encodes an exogenous nuclease.

Correction genomes useful in the AAV compositions disclosed herein generally comprise: (i) an editing element for editing a target locus in a MECP2 gene, (ii) a 5′ homology arm nucleotide sequence 5′ to the editing element having homology to a first genomic region 5′ to the target locus, and (iii) a 3′ homology arm nucleotide sequence 3′ to the editing element having homology to a second genomic region 3′ to the target locus, wherein the portion of the correction genome comprising the 5′ homology arm, editing element, and 3′ homology arm can be in the sense or antisense orientation relative to the MECP2 gene locus. The correction genome may further comprise a 5′ inverted terminal repeat (5′ ITR) nucleotide sequence 5′ to the 5′ homology arm nucleotide sequence, and a 3′ inverted terminal repeat (3′ ITR) nucleotide sequence 3′ to the 3′ homology arm nucleotide sequence.

The correction genomes useful in the AAV compositions disclosed herein are particularly advantageous since their use allows preservation of all native (endogenous) regulatory signal sequences, thereby ensuring that MeCP2 protein expression remains unaltered or as close as possible to wild-type MeCP2 expression level.

Editing elements used in the correction genomes disclosed herein can mediate insertion, deletion, or substitution of one or more nucleotides at the target locus.

In embodiments, when correctly integrated by homologous recombination at the target locus, the editing element corrects a mutation in a MECP2 gene back to the wild-type MECP2 sequence or a wild-type functional equivalent thereof. In embodiments, the editing element comprises a portion of a MECP2 coding sequence (e.g., a portion of a wild-type MECP2 coding sequence or a portion of a codon-altered MECP2 coding sequence).

In embodiments, the editing element comprises a wild-type or codon-altered sequence of exon 3 of a MECP2 gene. In embodiments, the editing element comprises a wild-type or codon-altered sequence of exon 4 of a MECP2 gene. In embodiments, the editing element comprises a wild-type or codon-altered sequence of exon 3 of a MECP2 gene and a wild-type or codon-altered sequence of exon 4 of a MECP2 gene.

In embodiments, codon-altered sequences as provided herein are single base substitutions or silent mutations, where the altered codon corresponds to the same amino acid in the MECP2 protein.

In embodiments, mutation-deficient MECP2 nucleic acid sequences as provided herein are sequences that encode a wild-type MeCP2 protein. In embodiments, a mutation-deficient MECP2 nucleic acid sequence includes the wild-type MECP2 nucleotide sequence. In embodiments, a mutation-deficient MECP2 nucleic acid sequence includes a codon-altered MECP2 nucleic acid sequence that differs from the wild type nucleotide sequence by including a different single base within a specific codon. In embodiments, the codon-altered MECP2 nucleic acid sequence encodes the same amino acid as in the wild-type MeCP2 protein sequence. In embodiments, a mutation-deficient MECP2 nucleic acid sequence includes a codon-optimized MECP2 nucleic acid sequence that differs from the wild type nucleotide sequence by including a different single base within a specific codon. In embodiments, the codon-optimized MECP2 nucleic acid sequence encodes the same amino acid as in the wild-type MeCP2 protein sequence. In embodiments, a “mutation-deficient” MECP2 nucleic acid sequence consists of any nucleotide sequences that encode the wild-type MeCP2 protein or a mutation-deficient MeCP2 protein that has the same amino acid sequence as the wild-type MeCP2 protein. In embodiments, a “mutation-deficient” MECP2 nucleic acid sequence comprises one or more different single bases within specific codons that force recombination outside the exon area while maintaining the correct protein sequence and codon usage. In embodiments, a “mutation-deficient” MECP2 nucleic acid sequence maintains the proper physiological level of functional MeCP2 proteins.

Thus, in embodiments, the AAV compositions disclosed herein comprise: (a) an AAV capsid; and (b) a correction genome comprising: (i) an editing element for editing a target locus in a MECP2 gene; (ii) a 5′ homology arm nucleotide sequence 5′ to the editing element having homology to a first genomic region 5′ to the target locus; and (iii) a 3′ homology arm nucleotide sequence 3′ to the editing element having homology to a second genomic region 3′ to the target locus.

In embodiments, the editing element comprises a MECP2 Exon 3 nucleic acid sequence that encodes a mutation-deficient MeCP2 Exon 3 protein sequence or portion thereof.

In embodiments, the editing element comprises a MECP2 Exon 4 nucleic acid sequence that encodes a mutation-deficient MeCP2 Exon 4 protein sequence.

In embodiments, the editing element comprises a MECP2 Exon 3 nucleic acid sequence that encodes a mutation-deficient MeCP2 Exon 3 protein sequence or portion thereof, and a MECP2 Exon 4 nucleic acid sequence that encodes a mutation-deficient MeCP2 Exon 4 protein sequence.

In embodiments, the Exon 3 nucleic acid sequence encodes a wild-type MeCP2 Exon 3 protein sequence or a fragment thereof. In embodiments, the Exon 3 nucleic acid sequence is a codon-altered nucleic acid sequence.

In embodiments, the Exon 4 nucleic acid sequence encodes a wild-type MeCP2 Exon 4 protein sequence or a fragment thereof. In embodiments, the Exon 4 nucleic acid sequence is a codon-altered nucleic acid sequence.

In embodiments, the editing element comprises a MECP2 Exon 3 nucleic acid sequence that comprises SEQ ID NO: 1.

In embodiments, the 5′ homology arm nucleotide sequence comprises a MECP2 intron 2 nucleic acid sequence. In embodiments, the MECP2 intron 2 nucleic acid sequence comprises SEQ ID NO: 2. In embodiments, the MECP2 intron 2 nucleic acid sequence comprises SEQ ID NO: 11.

In embodiments, the 3′ homology arm nucleotide sequence comprises a MECP2 intron 3 nucleic acid sequence. In embodiments, the 3′ homology arm nucleotide sequence comprises SEQ ID NO: 3. In embodiments, the 3′ homology arm additionally comprises a MECP2 Exon 4 nucleic acid sequence. In embodiments, the 3′ homology arm nucleotide sequence comprises SEQ ID NO: 4.

In embodiments, the editing element comprises a MECP2 Exon 4 nucleic acid sequence. In embodiments, the editing element comprises SEQ ID NO: 4.

In embodiments, the 5′ homology arm nucleotide sequence comprises a MECP2 intron 2, exon 3, and intron 3 nucleic acid sequence. In embodiments, the 5′ homology arm nucleotide sequence comprises SEQ ID NO: 5.

In embodiments, the 3′ homology arm nucleotide sequence comprises a MECP2 3′ untranslated (UTR) nucleic acid sequence. In embodiments, the 3′ homology arm nucleotide sequence comprises SEQ ID NO: 6. In embodiments, the 3′ homology arm nucleotide sequence comprises SEQ ID NO: 12.

The AAV capsid proteins that can be used in the AAV compositions disclosed herein include without limitation AAV capsid proteins and derivatives thereof of Clade A AAVs, Clade B AAVs, Clade C AAVs, Clade D AAVs, Clade E AAVs, and Clade F AAVs. In embodiments, the AAV capsid protein is a Clade F AAV capsid protein. Any AAV Clade F capsid protein or derivative thereof can be used in the AAV compositions disclosed herein.

In embodiments, the Exon 3 nucleic acid sequence and the Exon 4 nucleic acid sequences are codon-altered nucleic acid sequences.

Codon-altered sequences as provided herein are single base substitutions or silent mutations, where the altered codon corresponds to the same amino acid in the MeCP2 protein.

As disclosed herein, codon alteration ensures retention of endogenous regulatory sequences and occurrence of precise cross-over and maintains correct level of MeCP2 protein expression. SEQ ID NO: 7 and SEQ ID NO: 9 are examples of codon-altered sequences useful in the AAV compositions provided herein. SEQ ID NO: 8 is the wild-type MeCP2 amino acid sequence encoded by the codon-altered sequence of SEQ ID NO: 7, and SEQ ID NO: 10 is the MeCP2 amino acid sequence encoded by the codon-altered sequence of SEQ ID NO: 9.

In embodiments, the mutation is at nucleotides 22-24 of Exon 4. In embodiments, the mutation is at nucleotide 23 of Exon 4, resulting in a mutant TGT codon (cysteine), and consequent S134C mutation in the MECP2 protein. In embodiments, following AAVHSC editing, the mutant G is replaced with the wild type C nucleotide, thereby correcting the protein sequence to the wild-type MECP2 sequence.

In embodiments, an editing element as described herein comprises at least 0, 1, 2, 10, 100, 200, 500, 1000, 1500, or 2000 nucleotides. In embodiments, the editing element comprises or consists of 1 to 2000, 1 to 1000, 1 to 500, 1 to 200, 1 to 100, 1 to 50, or 1 to 10 nucleotides.

Homology arms used in the correction genomes disclosed herein can be directed to any region of the MECP2 gene or a gene nearby on the genome. The precise identity and positioning of the homology arms are determined by the identity of the editing element and/or the target locus.

Homology arms employed in the correction genomes disclosed herein are substantially identical to the genome flanking a target locus (e.g., a target locus in the MECP2 gene). In embodiments, the 5′ homology arm has at least about 90% (e.g., at least about 95%, 96%, 97%, 98%, 99%, or 99.5%) nucleotide sequence identity to a first region 5′ to the target locus. In embodiments, the 5′ homology arm has 100% nucleotide sequence identity to the first region. In certain embodiments, the 3′ homology arm has at least about 90% (e.g., at least about 95%, 96%, 97%, 98%, 99%, or 99.5%) nucleotide sequence identity to a second region 3′ to the target locus. In embodiments, the 3′ homology arm has 100% nucleotide sequence identity to the second region. In embodiments, the 5′ and 3′ homology arms are each at least about 90% (e.g., at least about 95%, 96%, 97%, 98%, 99%, or 99.5%) identical to the first and second regions flanking the target locus (e.g., a target locus in the MECP2 gene), respectively. In embodiments, the 5′ and 3′ homology arms are each 100% identical to the first and second regions flanking the target locus (e.g., a target locus in the MECP2 gene), respectively. In embodiments, differences in nucleotide sequences of the 5′ homology arm and/or the 3′ homology arm and the corresponding regions of the genome flanking a target locus comprise, consist essentially of, or consist of non-coding differences in nucleotide sequences.

The skilled worker will appreciate that homology arms do not need to be 100% identical to the genomic sequence flanking the target locus to be able to mediate integration of an editing element into that target locus by homologous recombination. For example, the homology arms can comprise one or more genetic variations in the human population, and/or one or more modifications (e.g., nucleotide substitutions, insertions, or deletions) designed to improve expression level or specificity. Human genetic variations include both inherited variations and de novo variations that are private to the target genome, and encompass simple nucleotide polymorphisms, insertions, deletions, rearrangements, inversions, duplications, micro-repeats, and combinations thereof. Such variations are known in the art, and can be found, for example, in the databases of dnSNP (see Sherry et al. Nucleic Acids Res. 2001; 29(1):308-11), the Database of Genomic Variants (see Nucleic Acids Res. 2014; 42 (Database issue):D986-92), ClinVar (see Nucleic Acids Res. 2014; 42 (Database issue): D980-D985), Genbank (see Nucleic Acids Res. 2016; 44 (Database issue): D67-D72), ENCODE (genome.ucsc.edu/encode/terms.html), JASPAR (see Nucleic Acids Res. 2018; 46(D1): D260-D266), and PROMO (see Messeguer et al. Bioinformatics 2002; 18(2):333-334; Farré et al. Nucleic Acids Res. 2003; 31(13):3651-3653), each of which is incorporated herein by reference. The skilled worker will further appreciate that in situations where a homology arm is not 100% identical to the genomic sequence flanking the target locus, homologous recombination between the homology arm and the genome may alter the genomic sequence flanking the target locus such that it becomes identical to the sequence of the homology arm used.

In embodiments, the 5′ homology arm has a length of about 50 to about 4500 nucleotides (e.g., about 100 to about 3000, about 200 to about 2500, about 300 to about 2000, about 400 to about 1500, about 500 to about 1000 nucleotides). In embodiments, the 5′ homology arm has a length of about 700 nucleotides. In embodiments, the 5′ homology arm has a length of about 600 nucleotides. In embodiments, the 5′ homology arm has a length of about 550 nucleotides. In embodiments, the 5′ homology arm has a length of about 500 nucleotides. In embodiments, the 5′ homology arm has a length of about 450 nucleotides. In embodiments, the 5′ homology arm has a length of about 400 nucleotides. In embodiments, the 5′ homology arm has a length of about 300 nucleotides. In embodiments, the 5′ homology arm has a length of about 200 nucleotides. In embodiments, the 5′ homology arm has a length of about 100 nucleotides. In embodiments, the 3′ homology arm has a length of about 50 to about 4500 nucleotides (e.g., about 100 to about 3000, about 200 to about 2500, about 300 to about 2000, about 400 to about 1500, about 500 to about 1000 nucleotides). In embodiments, the 3′ homology arm has a length of about 800 nucleotides. In embodiments, the 3′ homology arm has a length of about 800 nucleotides. In embodiments, the 3′ homology arm has a length of about 750 nucleotides. In embodiments, the 3′ homology arm has a length of about 700 nucleotides. In embodiments, the 3′ homology arm has a length of about 650 nucleotides. In embodiments, the 3′ homology arm has a length of about 600 nucleotides. In embodiments, the 3′ homology arm has a length of about 550 nucleotides. In embodiments, the 3′ homology arm has a length of about 500 nucleotides. In embodiments, the 3′ homology arm has a length of about 400 nucleotides. In embodiments, the 3′ homology arm has a length of about 300 nucleotides. In embodiments, the 3′ homology arm has a length of about 200 nucleotides. In embodiments, the 3′ homology arm has a length of about 100 nucleotides. In embodiments, each of the 5′ and 3′ homology arms independently has a length of about 50 to about 4500 nucleotides (e.g., about 100 to about 3000, about 200 to about 2500, about 300 to about 2000, about 400 to about 1500, about 500 to about 1000 nucleotides). In embodiments, each of the 5′ and 3′ homology arms has a length of about 800 nucleotides.

In embodiments, the 5′ and 3′ homology arms have substantially equal nucleotide lengths. In embodiments, the 5′ and 3′ homology arms have asymmetrical nucleotide lengths. In embodiments, the asymmetry in nucleotide length is defined by a difference between the 5′ and 3′ homology arms of up to 90% in the length, such as up to an 80%, 70%, 60%, 50%, 40%, 30%, 20%, or 10% difference in the length.

In embodiments, the correction genomes disclosed herein further comprise a 5′ inverted terminal repeat (5′ ITR) nucleotide sequence 5′ to the 5′ homology arm nucleotide sequence, and a 3′ inverted terminal repeat (3′ ITR) nucleotide sequence 3′ to the 3′ homology arm nucleotide sequence. ITR sequences from any AAV serotype or variant thereof can be used in the correction genomes disclosed herein. The 5′ and 3′ ITR can be from an AAV of the same serotype or from AAVs of different serotypes.

The AAV compositions disclosed herein are particularly advantageous in that they are capable of correcting one or more mutations in the MECP2 gene in a cell with high efficiency both in vivo and in vitro. In embodiments, the insertion efficiency of the editing element into the target locus is at least 2% (e.g., at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%) when the AAV is contacted in vitro in the absence of an exogenous nuclease with a population of cells under standard AAV transduction conditions. In embodiments, the insertion efficiency of the editing element into the target locus is at least 3%. In embodiments, the insertion efficiency of the editing element into the target locus is at least 4%. In embodiments, the insertion efficiency of the editing element into the target locus is at least 5%. In embodiments, the insertion efficiency of the editing element into the target locus is at least 6%. In embodiments, the insertion efficiency of the editing element into the target locus is at least 7%. In embodiments, the insertion efficiency of the editing element into the target locus is at least 8%. In embodiments, the insertion efficiency of the editing element into the target locus is at least 9%. In embodiments, the insertion efficiency of the editing element into the target locus is at least 10%. In embodiments, the insertion efficiency of the editing element into the target locus is at least 15%. In embodiments, the insertion efficiency of the editing element into the target locus is at least 20%. In embodiments, the insertion efficiency of the editing element into the target locus is at least 25%. In embodiments, the insertion efficiency of the editing element into the target locus is at least 30%. In embodiments, the insertion efficiency of the editing element into the target locus is at least 35%. In embodiments, the insertion efficiency of the editing element into the target locus is at least 40%. In embodiments, the insertion efficiency of the editing element into the target locus is at least 45%. In embodiments, the insertion efficiency of the editing element into the target locus is at least 50%. In embodiments, the insertion efficiency of the editing element into the target locus is at least 55%. In embodiments, the insertion efficiency of the editing element into the target locus is at least 60%. In embodiments, the insertion efficiency of the editing element into the target locus is at least 65%. In embodiments, the insertion efficiency of the editing element into the target locus is at least 70%. In embodiments, the insertion efficiency of the editing element into the target locus is at least 75%. In embodiments, the insertion efficiency of the editing element into the target locus is at least 80%. In embodiments, the insertion efficiency of the editing element into the target locus is at least 85%. In embodiments, the insertion efficiency of the editing element into the target locus is at least 90%. In embodiments, the insertion efficiency of the editing element into the target locus is at least 95%. In embodiments, the insertion efficiency of the editing element into the target locus is 100%.

In embodiments, the allelic frequency of insertion of the editing element into the target locus is at least 1% (e.g., at least 1%, 1.5%, 2%, 2.5%, 3%, 3.5%, 4%, 4.5%, 5%, 7.5%, 10%, 15%, 20%, 25%, 30%, 35%, 40%, 45%, 50%, 55%, 60%, 65%, 70%, 75%, 80%, 85%, 90%, or 95%) when the AAV is contacted in vitro in the absence of a exogenous nuclease with a population of cells under standard AAV transduction conditions. In embodiments, the allelic frequency of insertion of the editing element into the target locus is at least 1.5%. In embodiments, the allelic frequency of insertion of the editing element into the target locus is at least 2%. In embodiments, the allelic frequency of insertion of the editing element into the target locus is at least 2.5%. In embodiments, the allelic frequency of insertion of the editing element into the target locus is at least 3%. In embodiments, the allelic frequency of insertion of the editing element into the target locus is at least 3.5%. In embodiments, the allelic frequency of insertion of the editing element into the target locus is at least 4%. In embodiments, the allelic frequency of insertion of the editing element into the target locus is at least 4.5%. In embodiments, the allelic frequency of insertion of the editing element into the target locus is at least 5%. In embodiments, the allelic frequency of insertion of the editing element into the target locus is at least 7.5%. In embodiments, the allelic frequency of insertion of the editing element into the target locus is at least 10%. In embodiments, the allelic frequency of insertion of the editing element into the target locus is at least 15%. In embodiments, the allelic frequency of insertion of the editing element into the target locus is at least 20%. In embodiments, the allelic frequency of insertion of the editing element into the target locus is at least 25%. In embodiments, the allelic frequency of insertion of the editing element into the target locus is at least 30%. In embodiments, the allelic frequency of insertion of the editing element into the target locus is at least 35%. In embodiments, the allelic frequency of insertion of the editing element into the target locus is at least 40%. In embodiments, the allelic frequency of insertion of the editing element into the target locus is at least 45%. In embodiments, the allelic frequency of insertion of the editing element into the target locus is at least 50%. In embodiments, the allelic frequency of insertion of the editing element into the target locus is at least 55%. In embodiments, the allelic frequency of insertion of the editing element into the target locus is at least 60%. In embodiments, the allelic frequency of insertion of the editing element into the target locus is at least 65%. In embodiments, the allelic frequency of insertion of the editing element into the target locus is at least 70%. In embodiments, the allelic frequency of insertion of the editing element into the target locus is at least 75%. In embodiments, the allelic frequency of insertion of the editing element into the target locus is at least 80%. In embodiments, the allelic frequency of insertion of the editing element into the target locus is at least 85%. In embodiments, the allelic frequency of insertion of the editing element into the target locus is at least 90%. In embodiments, the allelic frequency of insertion of the editing element into the target locus is at least 95%. In embodiments, the allelic frequency of insertion of the editing element into the target locus is 100%.

Cells may include, but are not limited to, neurons, astrocytes, microganglial cells, lymphoblastoid cells, and fibroblasts. In embodiments, the insertion efficiency of the editing element into the target locus in the liver is at least 2% (e.g., at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%) when the AAV is administered to a subject in the absence of an exogenous nuclease or a nuclease sequence that encodes an exogenous nuclease under standard AAV administration conditions. In embodiments, the insertion efficiency of the editing element into the target locus in the liver is at least 3%. In embodiments, the insertion efficiency of the editing element into the target locus in the liver is at least 4%. In embodiments, the insertion efficiency of the editing element into the target locus in the liver is at least 5%. In embodiments, the insertion efficiency of the editing element into the target locus in the liver is at least 6%. In embodiments, the insertion efficiency of the editing element into the target locus in the liver is at least 7%. In embodiments, the insertion efficiency of the editing element into the target locus in the liver is at least 8%. In embodiments, the insertion efficiency of the editing element into the target locus in the liver is at least 9%. In embodiments, the insertion efficiency of the editing element into the target locus in the liver is at least 10%. In embodiments, the insertion efficiency of the editing element into the target locus in the liver is at least 15%. In embodiments, the insertion efficiency of the editing element into the target locus in the liver is at least 20%. In embodiments, the insertion efficiency of the editing element into the target locus in the liver is at least 25%. In embodiments, the insertion efficiency of the editing element into the target locus in the liver is at least 30%. In embodiments, the insertion efficiency of the editing element into the target locus in the liver is at least 35%. In embodiments, the insertion efficiency of the editing element into the target locus in the liver is at least 40%. In embodiments, the insertion efficiency of the editing element into the target locus in the liver is at least 45%. In embodiments, the insertion efficiency of the editing element into the target locus in the liver is at least 50%. In embodiments, the insertion efficiency of the editing element into the target locus in the liver is at least 55%. In embodiments, the insertion efficiency of the editing element into the target locus in the liver is at least 60%. In embodiments, the insertion efficiency of the editing element into the target locus in the liver is at least 65%. In embodiments, the insertion efficiency of the editing element into the target locus in the liver is at least 70%. In embodiments, the insertion efficiency of the editing element into the target locus in the liver is at least 75%. In embodiments, the insertion efficiency of the editing element into the target locus in the liver is at least 80%. In embodiments, the insertion efficiency of the editing element into the target locus in the liver is at least 85%. In embodiments, the insertion efficiency of the editing element into the target locus in the liver is at least 90%. In embodiments, the insertion efficiency of the editing element into the target locus in the liver is at least 95%. In embodiments, the insertion efficiency of the editing element into the target locus in the liver is 100%.

In embodiments, the allelic frequency of integration of the editing element into the target locus is at least 1% (e.g., at least 1%, 1.5%, 2%, 2.5%, 3%, 3.5%, 4%, 4.5%, 5%, 7.5%, 10%, 15%, 20%, 25%, 30%, 35%, 40%, 45%, 50%, 55%, 60%, 65%, 70%, 75%, 80%, 85%, 90%, or 95%) when the AAV is administered to a subject in the absence of a exogenous nuclease or a nuclease sequence that encodes an exogenous nuclease under standard AAV administration conditions. In embodiments, the allelic frequency of insertion of the editing element into the target locus is at least 1.5%. In embodiments, the allelic frequency of insertion of the editing element into the target locus is at least 2%. In embodiments, the allelic frequency of insertion of the editing element into the target locus is at least 2.5%. In embodiments, the allelic frequency of insertion of the editing element into the target locus is at least 3%. In embodiments, the allelic frequency of insertion of the editing element into the target locus is at least 3.5%. In embodiments, the allelic frequency of insertion of the editing element into the target locus is at least 4%. In embodiments, the allelic frequency of insertion of the editing element into the target locus is at least 4.5%. In embodiments, the allelic frequency of insertion of the editing element into the target locus is at least 5%. In embodiments, the allelic frequency of insertion of the editing element into the target locus is at least 7.5%. In embodiments, the allelic frequency of insertion of the editing element into the target locus is at least 10%. In embodiments, the allelic frequency of insertion of the editing element into the target locus is at least 15%. In embodiments, the allelic frequency of insertion of the editing element into the target locus is at least 20%. In embodiments, the allelic frequency of insertion of the editing element into the target locus is at least 25%. In embodiments, the allelic frequency of insertion of the editing element into the target locus is at least 30%. In embodiments, the allelic frequency of insertion of the editing element into the target locus is at least 35%. In embodiments, the allelic frequency of insertion of the editing element into the target locus is at least 40%. In embodiments, the allelic frequency of insertion of the editing element into the target locus is at least 45%. In embodiments, the allelic frequency of insertion of the editing element into the target locus is at least 50%. In embodiments, the allelic frequency of insertion of the editing element into the target locus is at least 55%. In embodiments, the allelic frequency of insertion of the editing element into the target locus is at least 60%. In embodiments, the allelic frequency of insertion of the editing element into the target locus is at least 65%. In embodiments, the allelic frequency of insertion of the editing element into the target locus is at least 70%. In embodiments, the allelic frequency of insertion of the editing element into the target locus is at least 75%. In embodiments, the allelic frequency of insertion of the editing element into the target locus is at least 80%. In embodiments, the allelic frequency of insertion of the editing element into the target locus is at least 85%. In embodiments, the allelic frequency of insertion of the editing element into the target locus is at least 90%. In embodiments, the allelic frequency of insertion of the editing element into the target locus is at least 95%. In embodiments, the allelic frequency of insertion of the editing element into the target locus is 100%. As used herein, the term “standard AAV administration conditions” refers to administration of an AAV intravenously at a dose of 1.5×10¹¹ or 1.5×10¹³ vector genomes per kilogram of body weight for a subject having the size and body shape of a mouse. A skilled worker will appreciate that the dose should be adjusted according to the size and body shape of the subject to achieve similar predicted efficacy. An exemplary dose conversion between species is provided by Nair et al. (2016) J. Basic Clin. Pharm. 7(2): 27-31, which is incorporated by reference herein in its entirety.

Any methods of determining the efficiency of editing of the MECP2 gene can be employed. In embodiments, individual cells are separated from the population of transduced cells and subject to single-cell PCR using PCR primers that can identify the presence of an editing element correctly integrated into the target locus of the MECP2 gene. Such method can further comprise single-cell PCR of the same cells using PCR primers that selectively amplify an unmodified target locus. In this way, the genotype of the cells can be determined. For example, if the single-cell PCR showed that a cell has both an edited target locus and an unmodified target locus, then the cell would be considered heterozygous for the edited MECP2 gene.

Additionally or alternatively, in embodiments, linear amplification mediated PCR (LAM-PCR), quantitative PCR (qPCR), or digital droplet PCR (ddPCR) can be performed on DNA extracted from the population of transduced cells using primers and probes that only detect edited MECP2 alleles. Such method can further comprise an additional qPCR or ddPCR (either in the same reaction or a separate reaction) to determine the number of total genomes in the sample and the number of unedited MECP2 alleles. These numbers can be used to determine the allelic frequency of integration of the editing element into the target locus.

Additionally or alternatively, in embodiments, the MECP2 locus can be amplified from DNA extracted from the population of transduced cells either by PCR using primers that bind to regions of the MECP2 gene flanking the genomic region encompassed by the correction genome, or by linear amplification mediated PCR (LAM-PCR) using a primer that binds a region within the correction genome (e.g., a region comprising an exogenous sequence non-native to the locus. The resultant PCR amplicons can be individually sequenced using single molecule next generation sequencing (NGS) techniques to determine the relative number of edited and unedited MECP2 alleles present in the population of transduced cells. These numbers can be used to determine the allelic frequency of integration of the editing element into the target locus.

### Pharmaceutical Compositions

In another aspect, the instant disclosure provides pharmaceutical compositions comprising an AAV as disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current ed., Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed. Amer. Pharmaceutical Assoc.

### Methods of Use

In embodiments, disclosed herein is a method for correcting a mutation in a MECP2 gene in a cell, the method comprising transducing the cell with anyone of the replication-defective adeno-associated virus (AAV) provided herein, wherein the cell is transduced without co-transducing or co-administering an exogenous nuclease or a nucleotide sequence that encodes an exogenous nuclease.

The disclosed method may be used in any cell of any organ. Exemplary organs include the central nervous system, the brain, the heart, the liver, and the lungs. Suitable cells include, but are not limited to, hepatocytes, endothelial cells, neuronal cells, glial cells, astrocytes, fibroblasts, β-lymphoblasts, dermal cells, osteocytes, mucosal cells, myocytes, blood cells, adipocytes, and connective tissue cells.

In embodiments, the cell is in a mammalian subject and the AAV is administered to the subject in an amount effective to transduce the cell in the subject.

Additionally provided herein, is a method of treating a subject having a disease or disorder associated with a MECP2 gene mutation, the method comprising administering to the subject an effective amount of anyone of the replication-defective adeno-associated viruses (AAVs) disclosed herein; wherein the AAV is administered to the subject without an exogenous nuclease or a nucleotide sequence that encodes an exogenous nuclease.

In embodiments, the disease or disorder is Rett syndrome. In embodiments, the disease or disorder is any disease or disorder associated with a mutation in the MECP2 gene.

In embodiments, the subject is a human subject.

An AAV composition disclosed herein can be administered to a subject by any appropriate route including, without limitation, intravenous, intraperitoneal, intraarticular, intraparenchymal, intraspinal, subcutaneous, intramuscular, intranasal, topical or intradermal routes. In certain embodiments, the composition is formulated for administration via intravenous injection or subcutaneous injection.

### EXAMPLES

### Background

Rett Syndrome (RTT) is a debilitating and challenging genetic neurodevelopmental disorder observed almost exclusively in females, and is caused by heterozygous, de novo mutations in the MECP2 gene, located on chromosome Xq28. These mutations lead to a deficiency of the wild-type MECP2 protein in all cells including neurons. Males born with the MECP2 mutations rarely survive due to the presence of a single X-chromosome. Females with Rett Syndrome display random X-chromosome inactivation and are mosaic for MECP2 expression in all tissues. A progressive loss of motor skills and speech, seizures and intellectual disability is observed in children with Rett Syndrome.

MeCP2 is a 486-amino acid or 498-amino acid nuclear protein that functions as a methylation reader and regulates expression of thousands of genes through chromatin compaction at methylated sites and interaction with transcriptional regulators. The protein contains a methyl binding domain which binds to DNA, and a transcriptional repressor domain, with the C-terminal portion containing the NCoR/SMRT interaction domain. MECP2 is universally expressed, but the highest levels are observed in neurons. Over 600 distinct mutations in the MECP2 gene have been reported in patients with RTT, with almost all occurring within exons 3 and 4, and mapping to the MBD and C-terminus of TRD. Missense mutations account for approximately 70% of RTT cases. It is a direct result of the loss of MeCP2 function.

RTT is caused by mutations in the X-linked MECP2 gene, leading to deficiency of the wild type MECP2 protein in neurons. However, overexpression of MECP2 also results in a severe neurodevelopmental disorder, demonstrating the critical importance of MeCP2 gene dosage. Thus, MeCP2 gene dosage presents a “Goldilocks problem” and poses a significant challenge to traditional gene therapy approaches. There are currently no available therapies for RTT.

To overcome these challenges, we explored the ability of the nonpathogenic, replication-defective stem cell-derived adeno-associated virus (AAVHSC) to carry out high-fidelity, precise, homologous recombination-based seamless genome editing to correct pathogenic mutations associated with Rett Syndrome. Importantly, our editing strategy preserves all regulatory elements associated with the MECP2 gene, allowing physiologic expression and overcoming a major obstacle in genetic medicine.

The recombinant AAV vectors disclosed herein mediate highly efficient gene editing or gene transfer in vitro and in vivo. The following examples demonstrate the efficient correction of mutations in the MECP2 gene. These examples are offered by way of illustration, and not by way of limitation.

### Example 1: Generation of Codon Altered Correction Sequences to Precisely Control Location of Cross-Over Events for Homologous Recombination and Maximal Correction of Mutations

### Rationale for Codon Alteration

In AAVHSC mediated gene editing, a genomic segment carrying the defective mutation is replaced by a “correction”/“replacement” sequence specified in the editing vector genome. Typically, these sequences are bounded on the 5′ and 3′ ends by homology arms (HA), which specify the exact genomic location to be edited. Often, the correction sequences represent either the wildtype or a codon optimized/altered copy of the corresponding genomic segment. Replacement of the mutant sequence with the Correction Sequence then restores the normal function of the newly corrected version of the original mutant gene. This process is mediated by homologous recombination (HR). HR is directed by left and right homology arms that are identical (homologous) to specific locations in genomic DNA. The process of HR is initiated by the crossover events that are located within the regions of homology between the genome and the editing vector. The location of crossover events is stochastic and can occur anywhere within the stretch of homology between the genome and the vector.

When the Correction Sequence is identical to the genome, this creates an extra region of homology in addition to the HAs. In this case, we have observed that crossover events can occur at any location along the stretch of homology. Our observations suggest that the probability of recombination is directly proportional to the distance from the distal ends of the HAs, with the greatest frequency of crossovers observed at or near the center of the homology region. The frequency of crossovers decreases as we move outwards from the center.

Most pathogenic mutations are usually scattered at multiple locations throughout genic regions. Thus, if they are located closer to the center of the homology region, there will be a higher probability of correction. However, if they are located closer to the periphery of the homology region, the probability of correction will be lower, and editing may not result in correction of the defective allele.

To overcome this problem, we reasoned that we could use the degeneracy of the genetic code to force recombination to occur outside the Correction Sequence and solely within the HAs, thereby ensuring that all mutations in the region between the HAs will be corrected. We accomplished this by replacing the Correction Sequence with a different synonymous nucleotide sequence that encodes the same wild type amino acid. This may be accomplished using a process known as codon optimization. Codon optimization results in an altered DNA sequence but maintains the wild type amino acid sequence. This serves two purposes: 1. it prevents unwanted recombinations within the coding region and 2. may increase expression of the corrected protein.

However, for certain tightly regulated proteins like MeCP2, the expression level is critical to avoid toxicity and to ensure proper cellular functions. Either too much or too little MeCP2 expression is detrimental, and both are associated with pathogenesis.

To overcome this problem, we designed a different strategy to design “Codon Altered” Correction Sequences. Here we again used the degeneracy of the genetic code to alter the nucleotide sequence of exons to be corrected. However, the Codon Altered Correction Sequences were designed such that codon usage and distribution of the coding exons reflected the original physiologic codon usage and therefore is expected to maintain the same expression level as the endogenous protein. Using this strategy, we can now force recombination to occur only within the HAs, instead of within the coding regions while also maintaining physiologic expression of the edited protein which now has the wild type amino acid sequence (as opposed to the mutant protein).

In the genetic code, 64 combinations of triplet codons generate redundancy to encode 20 amino acids plus stop codons. Apart from methionine and tryptophan which are each encoded by a single codon, all other amino acids and stop codons are encoded by more than one codon with serine, arginine and leucine having the most diversity at six. Despite encoding the same amino acid, frequency of codon usage is not uniform within all tissues. Some variations can range as much as 0.07% to 35%. This variation in codon usage in conjunction with the abundance of corresponding tRNAs in different cell types enables fine tuning of the protein production in different tissues and more critically, ensures proper protein folding, which is important for function.

### Example 2: How Codon Alteration was Achieved

The goal of Codon Alteration is to change the DNA protein coding sequence while maintaining the same codon usage frequency as the wildtype thereby maintaining the physiologic protein expression level. For each amino acid within the coding exon, we counted the total number of each amino acid and the usage frequency of each codon. Our goal was to use alternate codons for which the codon usage and therefore tRNAs, existed at same frequency and ratio as wildtype for each codon used. For most of the amino acids that are encoded by two synonymous codons, including phenylalanine, tyrosine, asparagine, lysine, glutamic acid, cysteine and aspartic acid, the usage frequencies of the two codons are within 10%. In these cases, we simply changed the codons from the original to the other. For example, each of the TTT (46%) codon was changed to TTC (54%) and vice versa for phenylalanine. For codons that have greater than 10% usage frequency, such glutamine, the change to another codon is restricted by the overall ratio of each. For example, if in exon 4 there are 7 CAA (27%) codons and 4 CAG (73%) codons, only 4 of the CAA codons were changed to CAG and the other 3 were left unchanged in order to maintain the overall ratio of 7 CAA to 4 CAG codons. For those amino acids that are encoded by more than 3 different synonymous codons, each codon was replaced by a different synonymous codon with the closest frequency. For example, a CAA (28%) codon would be changed to CCT (29%) but not to CCG (11%), as the latter has a much lower usage frequency. No changes were made to codons with usage frequency of less than 15% since these may represent positions of potential translational pausing which is a critical step in ensuring proper protein folding. In addition, the two codons located at the 5′ and 3′ exon-intron junctions were not altered to ensure proper splicing.

### Example 3: Design of Editing Vectors

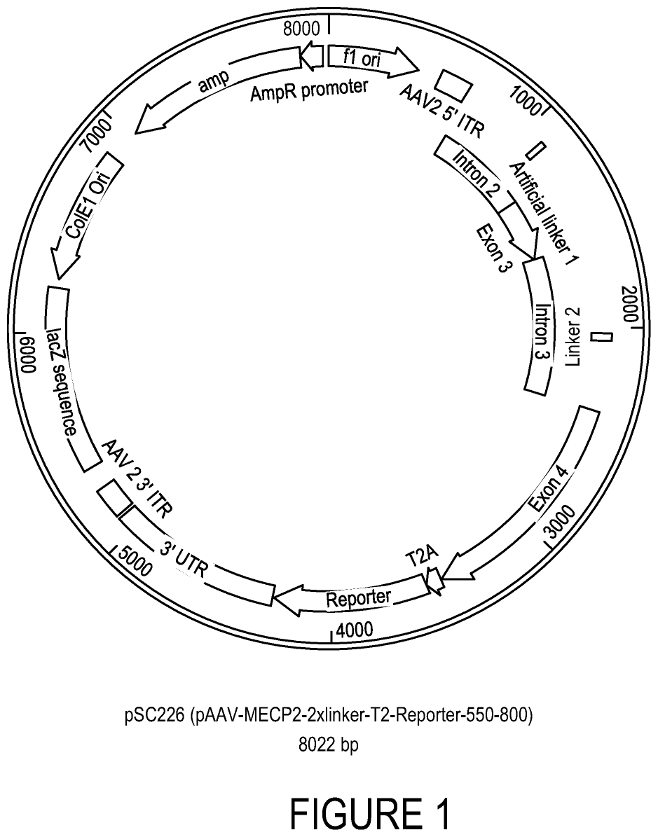

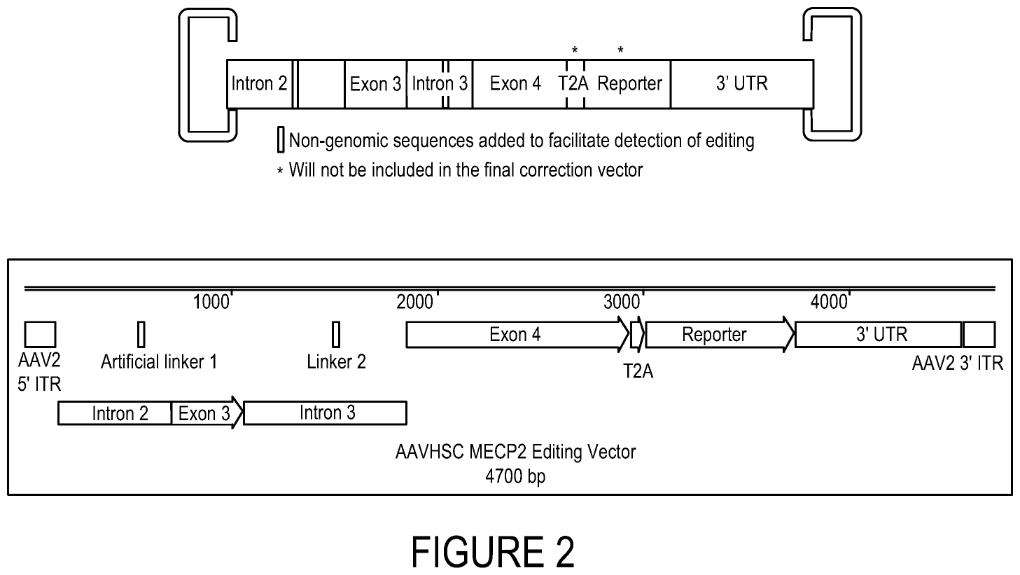

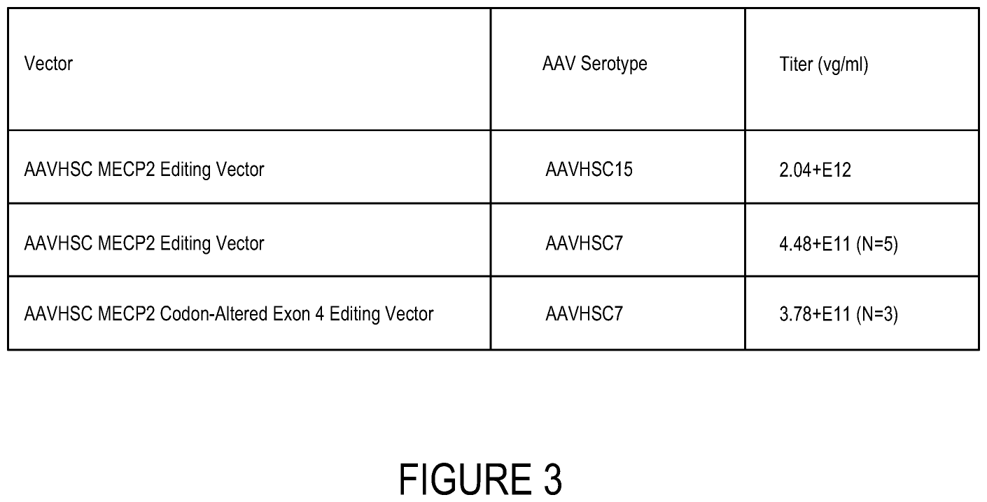

We designed AAVHSC-based editing vectors to edit the MECP2 gene to correct mutations in exons 3 and 4, which account for approximately 90% of the mutations associated with RTT. In addition to correcting mutations in exons 3 and 4, the initial MECP2 editing vector was designed to insert a promoterless Venus open reading frame immediately (ORF) downstream of exon 4. The Venus ORF was preceded by a T2A sequence to allow independent translation (FIGS. 1 and 2). To facilitate detection of editing, this vector also encoded a novel “Linker Sequence 1” in intron 2 and a ‘Linker sequence 2’ in intron 3. The MECP2 editing vector was packaged in AAVHSC7 and AAVHSC15. A representative set of titers after purification in shown in FIG. 3.

### List of AAV MECP2 Editing Vectors Containing Codon Altered MECP2

vSC418

vSC418 is an AAV vector for editing Exon 4 of the mouse MECP2 gene. It contains: left homology arm (LHA) that consists of 231 bp of wild type Exon3 and 520 bp of wild type Intron 3 sequence. A 33 bp artificial linker 2 is inserted into Intron 3 for detection of edited alleles. A Codon Altered Exon 4 that maintains the amino acid sequence and codon usage frequency as the wild type gene. A T2A self-cleaving peptide sequence and reporter gene firefly luciferase. Right homology arm (RHA) that consists of 800 bp of wild type non-coding Exon 4 sequence. AAV2 5′ and 3′ ITR sequences.

vSC419

vSC419 is an AAV vector for editing Exon 3 of the human MECP2 gene. It contains: a left homology arm (LHA) that consists of 549 bp of wild type Intron 2 with 28 bp artificial Linker 1 sequence for detection of edited alleles. A Codon Altered Exon 3 that maintains the wild type amino acid sequence and codon usage frequency. Wild type Intron 3 that contains 33 bp artificial Linker 2 sequence for detection of edited alleles. Wild type coding region of Exon 4. A T2A self-cleaving peptide sequence and a Venus reporter open reading frame. A right homology arm (RHA) that consists of 800 bp of wildtype non-coding Exon 4 sequence. AAV2 5′ and 3′ ITR sequences. vSC420

vSC420 is an AAV vector for editing Exon 4 of human MECP2 gene. It contains: left homology arm that consists of i) 549 bp of wild type Intron 2 with 28 bp artificial Linker 1 sequence; ii) 349 bp of wildtype Exon 3; iii) 789 bp of wild type Intron 3 with 33 bp artificial Linker 2. A Codon Altered coding region of Exon 4 that maintains amino acid sequence and codon usage frequency. A T2A self-cleaving peptide sequence and a Venus reporter open reading frame. A right homology arm (RHA) that consists of 800 bp of wild type non-coding Exon 4 sequence. E. AAV2 5′ and 3′ ITR sequences.

vSC433

vSC433 is an AAV vector for editing Exon 3 and Exon 4 of the human MECP2 gene. It contains: a left homology arm that consists of 923 bp of wild type Intron 2 with 28 bp artificial Linker 1. A codon altered Exon3 and a codon altered coding region of Exon 4. A T2A self-cleaving peptide sequence and a Venus reporter open reading frame. A right homology arm that consists of 800 bp of wild type non-coding Exon 4 sequence. AAV2 5′ and 3′ ITR sequences.

vSC414

vSC414 is an AAV vector for editing Exon 4 of mouse MECP2 gene. It contains: a left homology arm (LHA) that consists of 273 bp of wild type Exon 3, 520 bp of wild type Intron 3 sequence with a 33 bp artificial linker 2 inserted into Intron 3 for detection of edited alleles by PCR, and 1075 bp of wild type Exon 4. A T2A self-cleaving peptide sequence and firefly luciferase reporter open reading frame. A right homology arm (RHA) that consists of 782 bp of wildtype non-coding Exon 4 sequence. AAV2 5′ and 3′ ITR sequences.

### Example 4: Editing of the MECP2 Gene in RTT Patient Cells

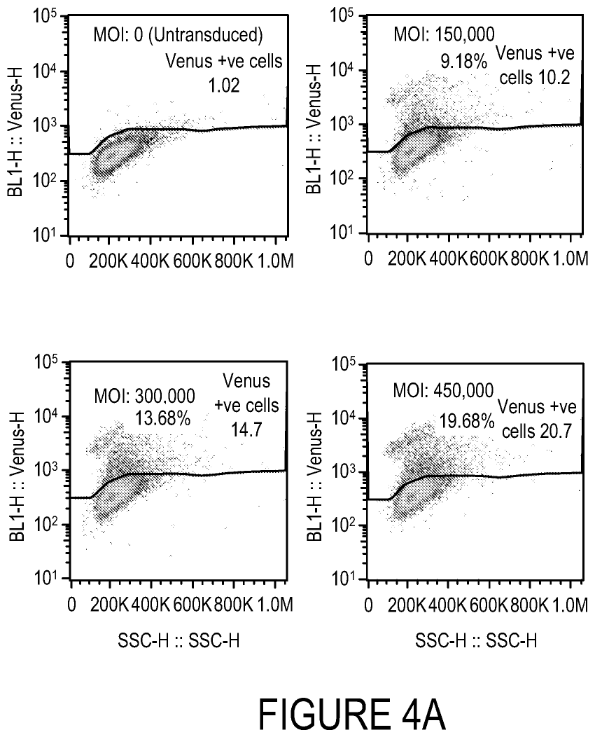

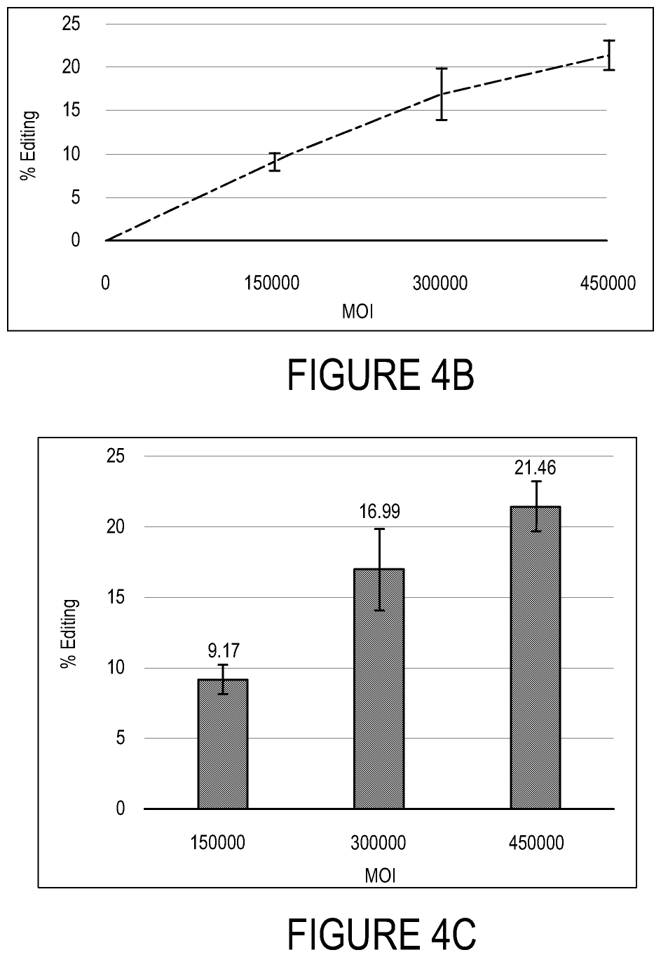

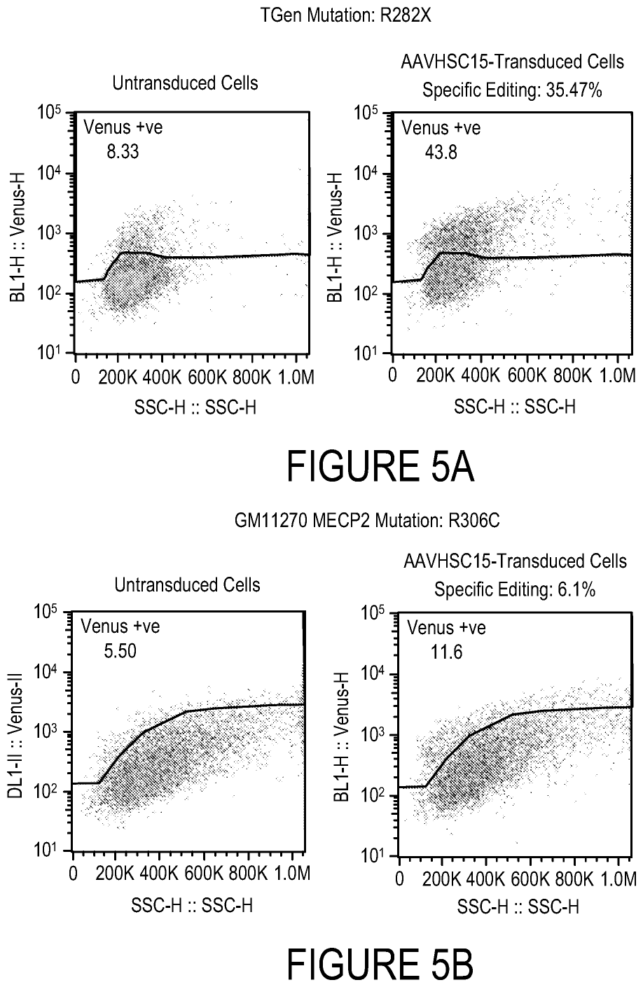

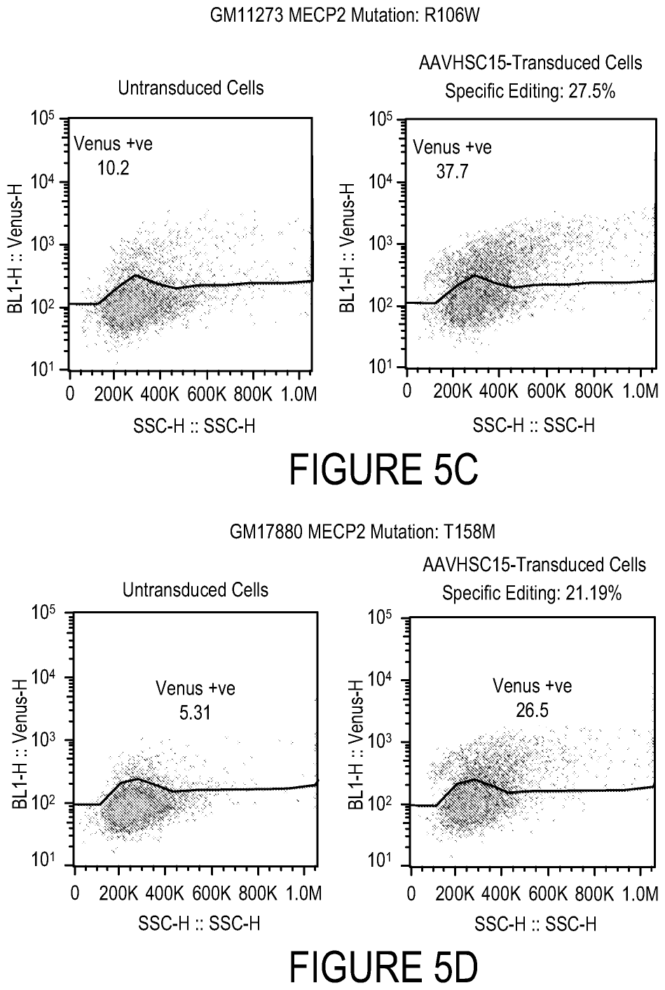

We next transduced primary fibroblasts derived from a patient with RTT carrying the heterozygous R282X mutation. Flow cytometric analyses 48 hours after transduction indicated specific Venus expression (FIG. 5A). Dose response experiments indicated that the frequency of editing increased linearly with increasing multiplicity of infection (FIGS. 4B, 4C).

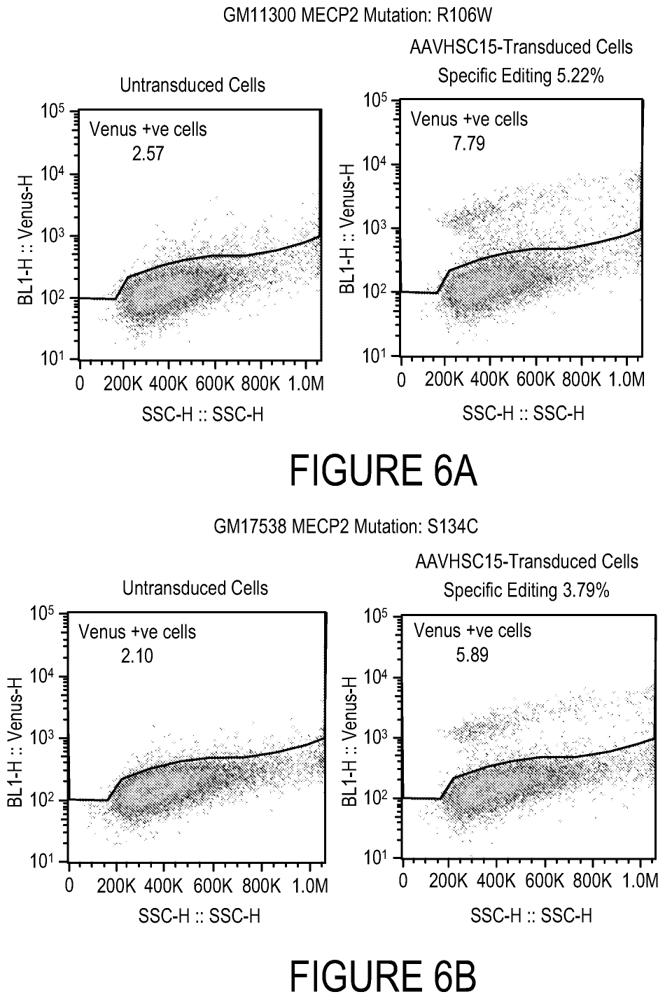

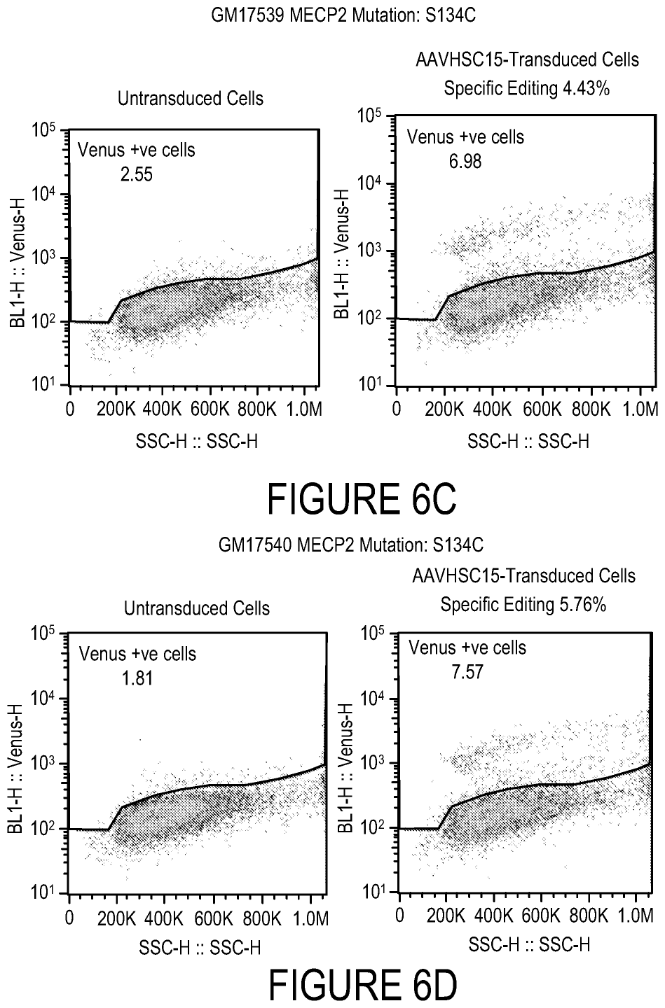

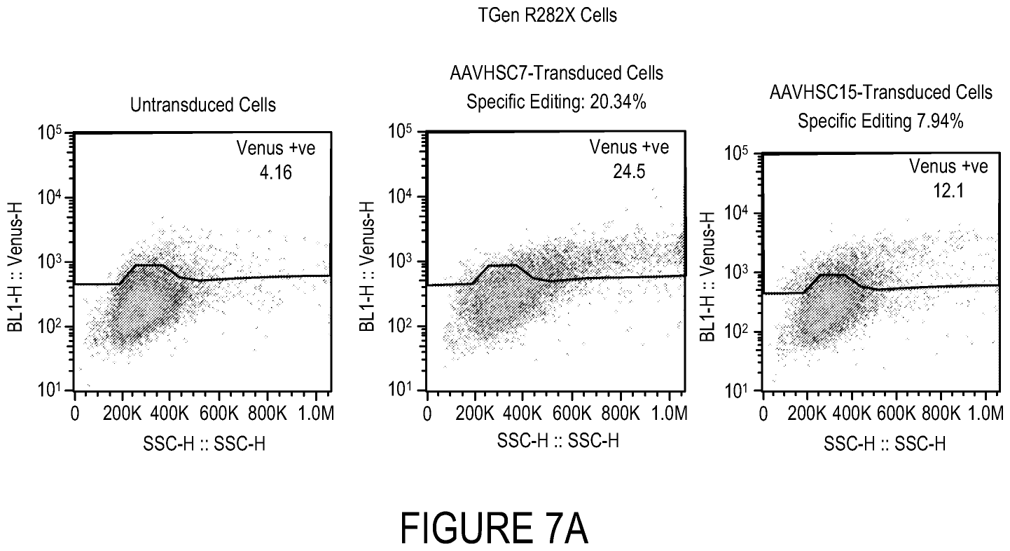

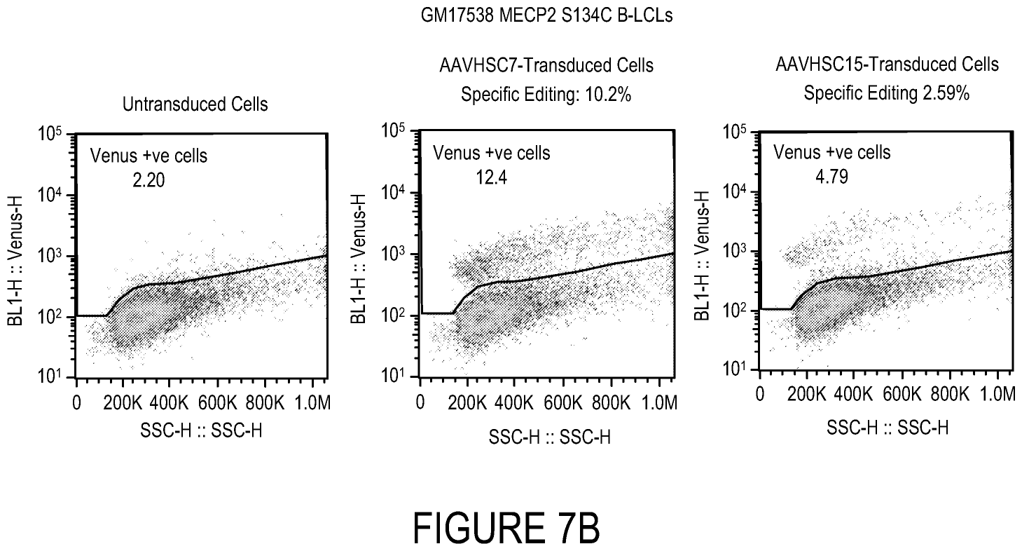

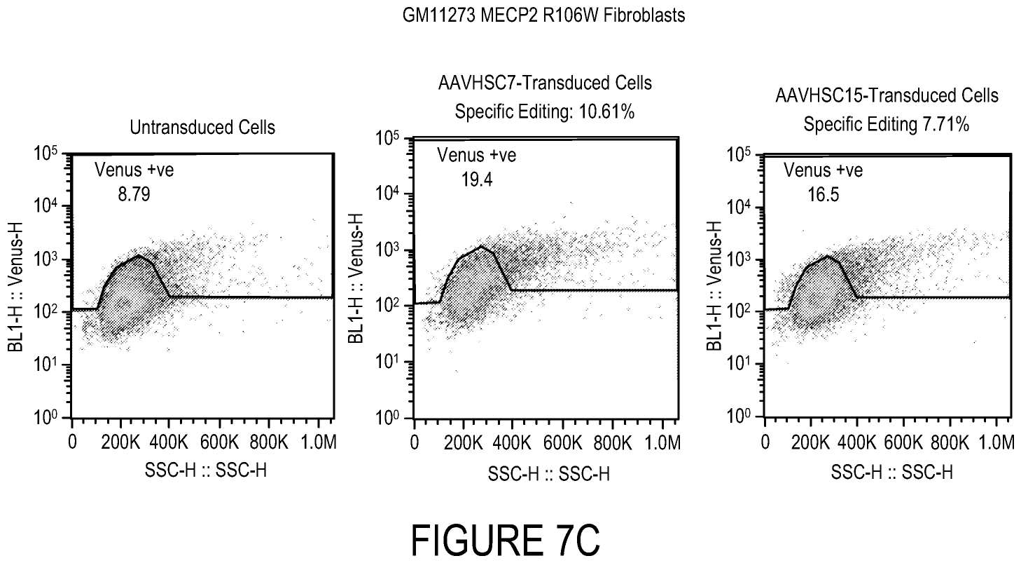

Editing of the MECP2 gene was next evaluated in a panel of primary fibroblasts and EBV-immortalized B lymphoblasts (B-LCLs) derived from eight patients with Rett Syndrome (FIGS. 5, 6). Specific editing was observed in each cell line analyzed and ranged from 3.79% to 35.47% (FIGS. 6, 7, 8, 9). Venus expression in cells from each patient analyzed demonstrated the feasibility of our editing strategy to edit the MECP2 gene to correct mutations associated with Rett Syndrome.

We evaluated cells from three members of a single family, all of whom carry the S134C mutation. This analysis included an asymptomatic mother and one affected son and one affected daughter. Editing was observed in all three cell lines. Importantly, editing of the cells derived from the son confirmed that the X chromosome carrying the mutant MECP2 can be successfully edited.

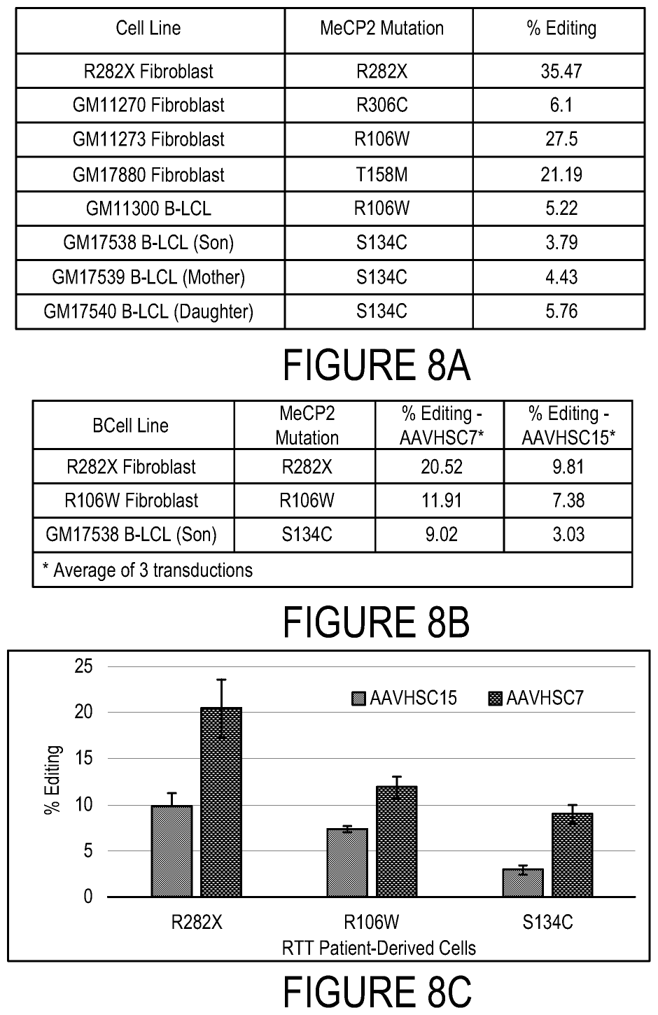

Comparison of MECP2 editing in R282X and in GM17538 cells with AAVHSC7 and AAVHSC15 revealed that both vectors edited both sets of cells. With AAVHSC7, 20.34% of R282X and 10.2% of GM17538 cells were edited, while 7.94% and 2.59% were edited AAVHSC15, respectively (FIGS. 8A-B). These results indicated that different AAVHSC serotypes, edit at different efficiencies.

### Example 5: Confirmation of MECP2 Editing at the Sequence Level

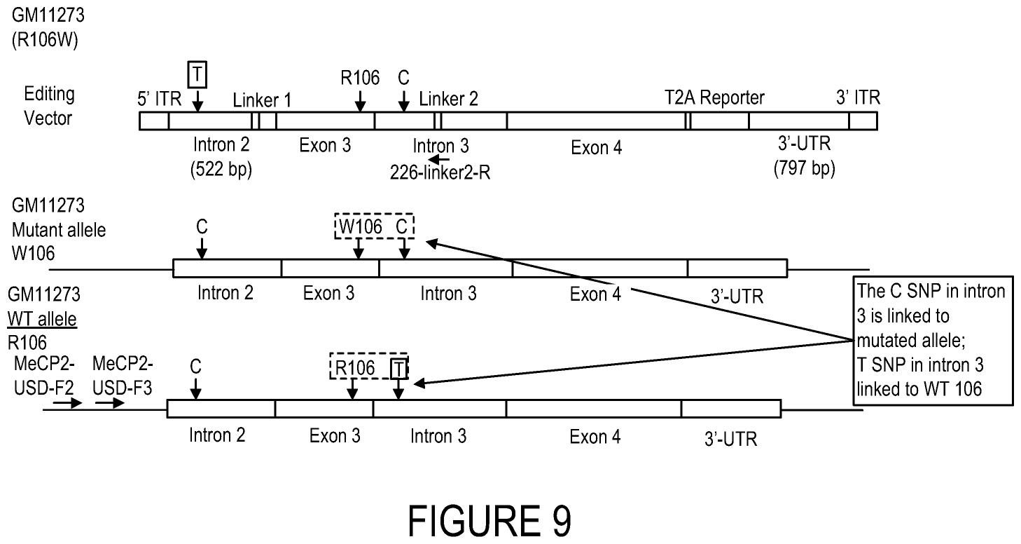

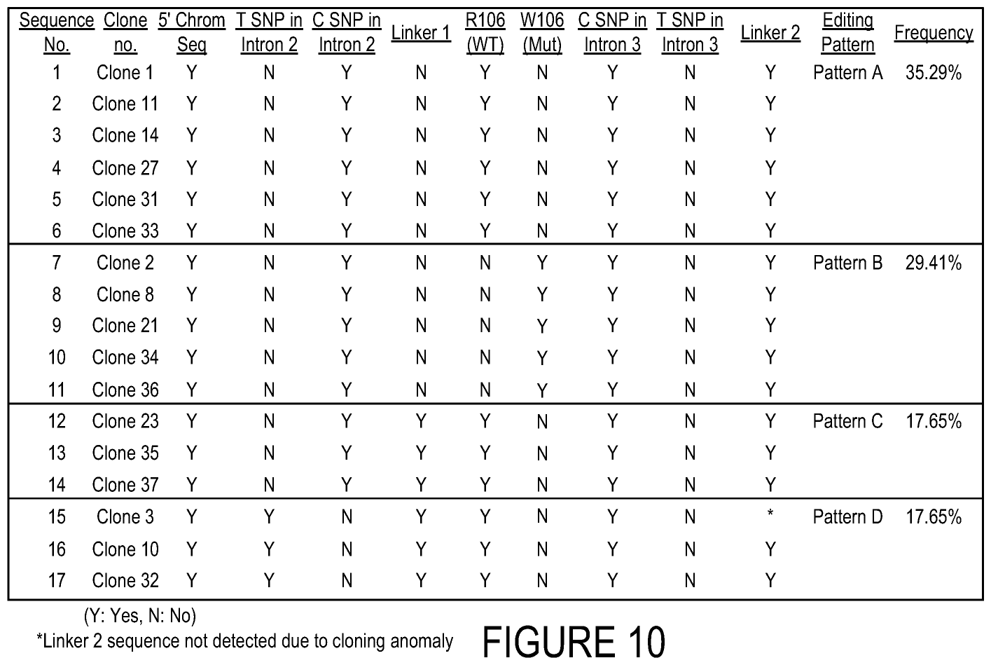

We next evaluated editing of the MECP2 gene at the sequence level in the genomic DNA isolated from AAVHSC-transduced RTT patient-derived cells. FIG. 10 shows the maps of the editing vector and the mutant and wild type MECP2 alleles in GM11273 cells derived from a patient with a heterozygous R106W mutation in MeCP2. Sequencing of unedited patient-derived cells indicated the existence of two single nucleotide polymorphisms (SNP), a T/C located in Intron 2 and another C/T SNP located in intron 3 (FIG. 9).

To confirm editing at the sequence level, we performed targeted integration (TI) assays. Using a chromosome-specific primer that hybridized to a sequence external to the MECP2 homologous region of the editing vector and a vector-specific Linker 2 primer, we amplified the 5′ (left) junction of the edited MECP2 locus (FIG. 9). The amplicon was cloned, and 17 clones were analyzed by Sanger sequencing. The results are summarized in FIG. 10.

### Sequence Analysis of the Edited MECP2 Gene in AAVHSC15-Transduced GM11273 (R106W) Fibroblasts Genomic DNA.

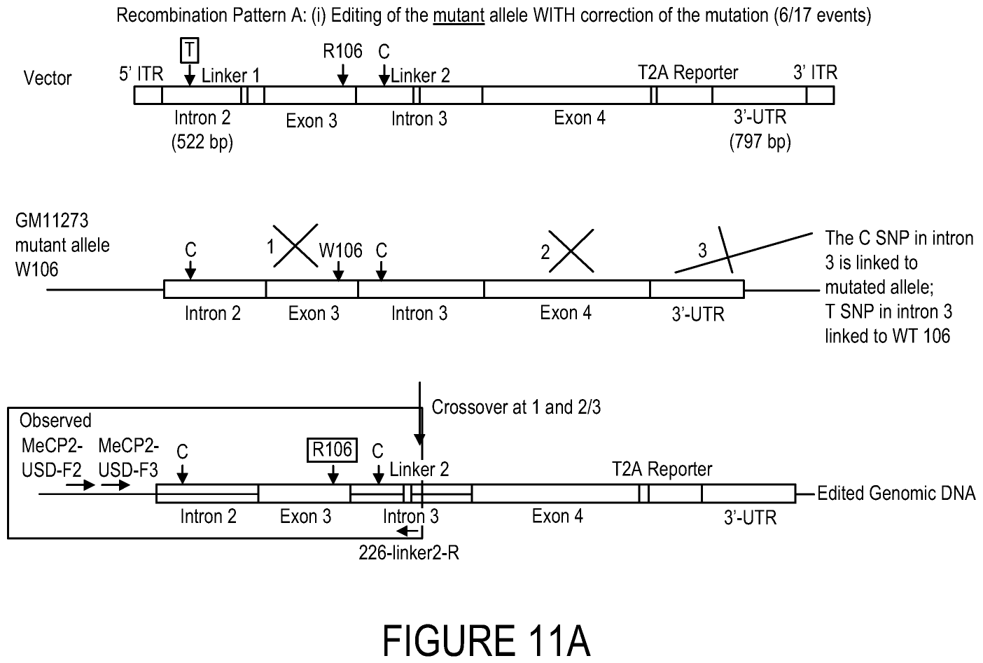

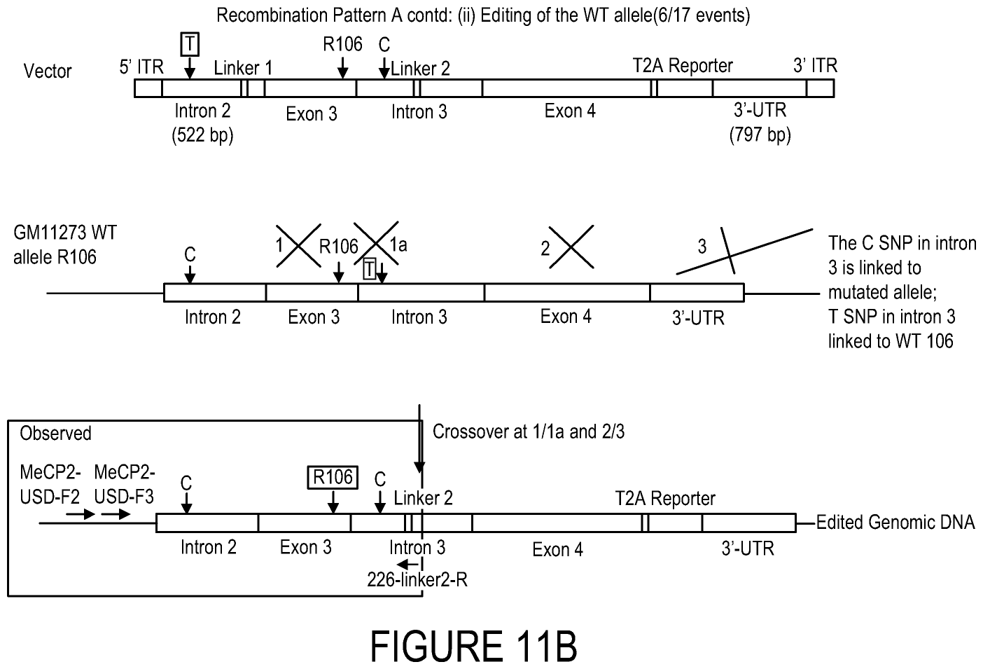

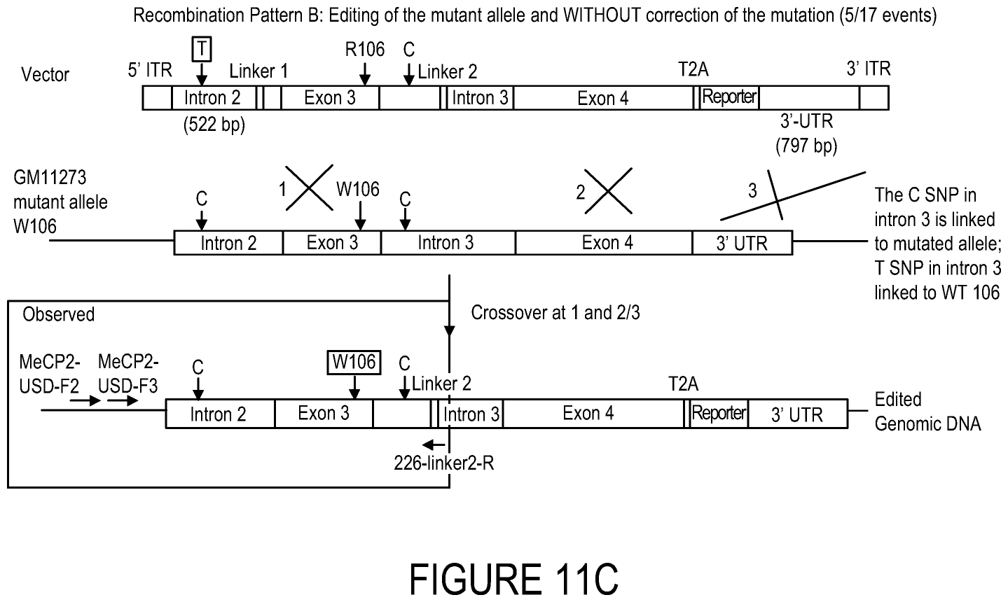

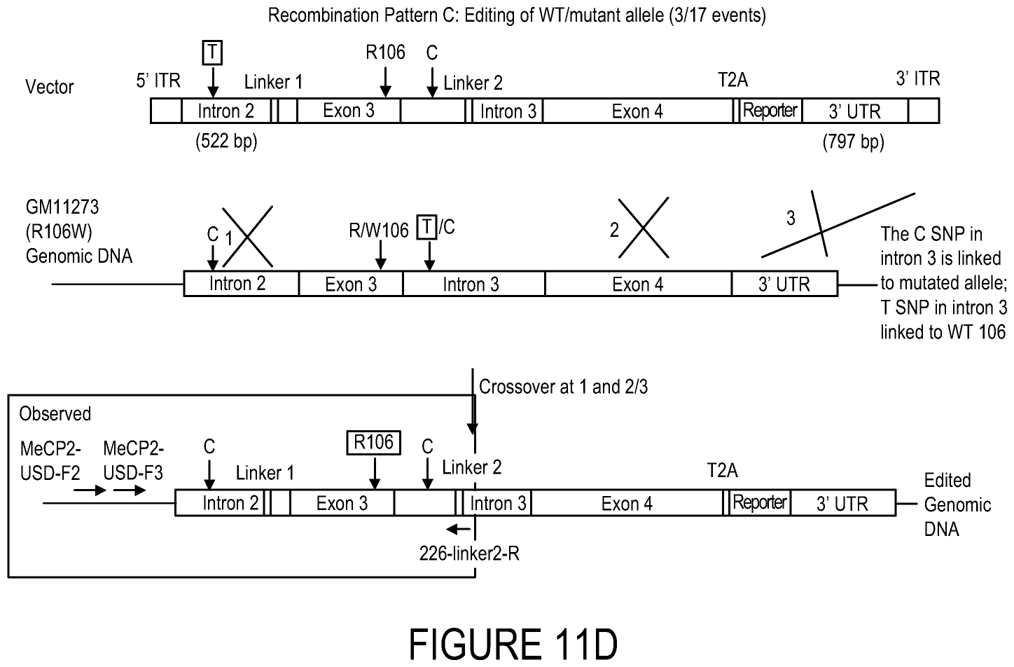

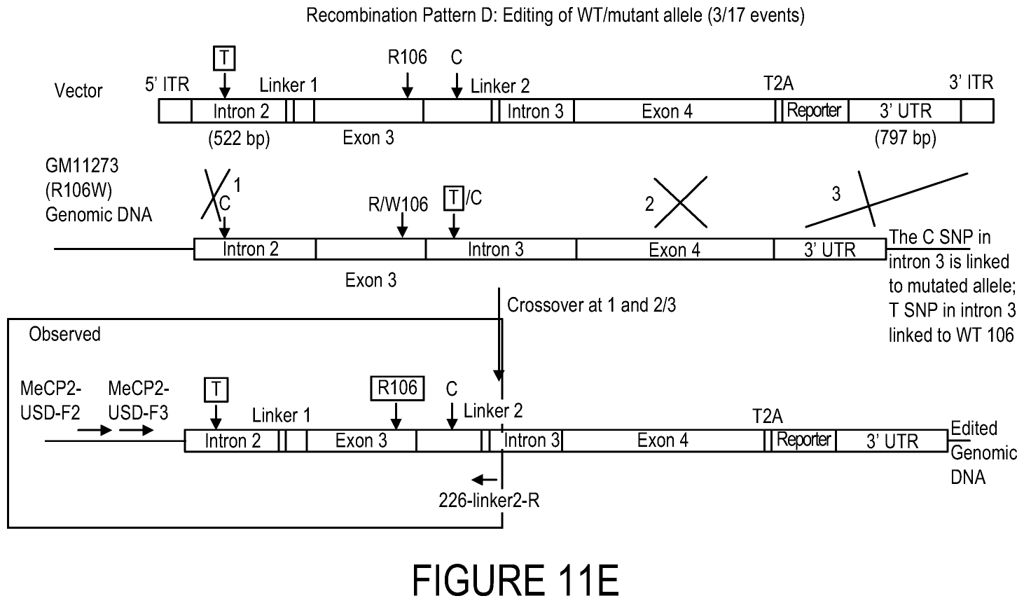

GM11273 cells were transduced with AAVHSC15 MECP2 editing vector and after 48 h, the cells were harvested to extract genomic DNA. Targeted integration (TI) assays were performed using a genomic primer complementary to genomic sequences upstream of homology arm (HA) and a reverse primer annealing to linker 2 sequence. The amplified product was cloned, and individual clones were sequenced. The sequencing of clones revealed different recombination patterns that are represented in FIG. 11. The sequencing of clones revealed the correction of the mutation at nucleotide 289 of exon 3 in 12 out of 17 clones, where the corrected codon CGG encodes for amino acid arginine. The sequences of clones exhibiting different recombination patterns are shown below.

### Correction of the R106W Mutation (Exon 3)

(A) Crossover pattern A (Clones 1, 11, 14, 27, 31, 33)
(SEQ ID NO: 18)
GCTCACAGTAAGGATGCCTAGATGGGGTTACCTGTTGTCAAGATAAGGAAACTGAA
GCACAGAATGCTGAGGTCATTTGCTGGGTTCATGTTTGGAAAGCGGCAAAGGATTTC
AGTGCAGGTTGGCTGGCTCCAAACCTGTGTGTGCTTTCCATGACACTGTACTGTGTG
CCTCATTGAGCCTCATTCTAGAAAACCAAAAACACACCCAAGGCCCGGCCTTCACA
AAGGAGACCCCTCCCCCATTTGGCTCCCTTTCCAGCAGTCGACGGCCTCTTGTCAGC
CATCGAGCCCAGAGTCCCTTGAAGTGCGACTCATGCTGGGGTGGTATGCTCAGGAGC
CGCAGTGTTTCCGCTCAGAGGAAAGGGCTCTGATTCTCCTGCAGTGCTAGGAGACTT
GTGGGTGGCCACAGTGCAGGTCAGGCACACCGGCCAGCACCACCCACAGCCCAAAT
TCCTAAAGAAATATTTGGGTCCCAGCTTGGCCCGAGTCTCTGTTGTCCTGGGGAAGG
ACATCAAGATCTGAGTGTATGATGGCCTGGGGCCTTGCATGTGGTGGGGGTCCAAGC
CTGCCTCTGCTCACTTGTTCTGCAGACTGGCATGTTCTCTGTGATACTTACATACTTG
TTTAACACTTCAGGGAAGAAAAGTCAGAAGACCAGGACCTCCAGGGCCTCAAGGAC
AAACCCCTCAAGTTTAAAAAGGTGAAGAAAGATAAGAAAGAAGAGAAAGAGGGCA
AGCATGAGCCCGTGCAGCCATCAGCCCACCACTCTGCTGAGCCCGCAGAGGCAGGC
AAAGCAGAGACATCAGAAGGGTCAGGCTCCGCCCCGGCTGTGCCGGAAGCTTCTGC
CTCCCCCAAACAGCGGCGCTCCATCATCCGTGACCGGGGACCCATGTATGATGACCC
CACCCTGCCTGAAGGCTGGACACGGAAGCTTAAGCAAAGGAAATCTGGCCGCTCTG
CTGGGAAGTATGATGTGTATTTGATCAAGTAAGTAAGAGCAACTCCTATCTCTACAG
GGCAGGGAGGGCAGGGACAAGGATCCCTCATGGAGCAGGAAAATGTATGTGCCCA
GGGTGGGGTCGGGGGGAACATAAACAATGAACACTGAGACCAGGTGTGCTTGAAAT
GACCGTGTACAGAGGTCGCTGCCCTGAGTGGGAAGTTCTCAAGGTAGCAGGCCCTCT
ATCCTCTCCACACCTCAAGTCTTTATCTGGGGATGGAATAGCTGCGGAAGCAGAGGA
ACTTGCAGAGCCAGGGGTTCAGAGGGGTGAAGAAGCATGTTTCAGTTCTGCCTTTTA
AATGATCCCAAAAAGGTTAGCAGTTTTCAAATGACATTTGCAGACAGCCTCATTTAA
TTCCATGAGAAGGGTGAGCAAAGGATTATCTTGTTGAAACTGATTCCTGGAGAGACT
GAGCACACTAGTGACTACAAAGACCATGACGG
(B) Crossover pattern B (Clones 2, 8, 21, 34, 36)
(SEQ ID NO: 19)
GGGTTACCTGTTGTCAAGATAAGGAAACTGAAGCACAGAATGCTGAGGTCATTTGCT
GGGTTCATGTTTGGAAAGCGGCAAAGGATTTCAGTGCAGGTTGGCTGGCTCCAAACC
TGTGTGTGCTTTCCATGACACTGTACTGTGTGCCTCATTGAGCCTCATTCTAGAAAAC
CAAAAACACACCCAAGGCCCGGCCTTCACAAAGGAGACCCCTCCCCCATTTGGCTC
CCTTTCCAGCAGTCGACGGCCTCTTGTCAGCCATCGAGCCCAGAGTCCCTTGAAGTG
CGACTCATGCTGGGGTGGTATGCTCAGGAGCCGCAGTGTTTCCGCTCAGAGGAAAG
GGCTCTGATTCTCCTGCAGTGCTAGGAGACTTGTGGGTGGCCACAGTGCAGGTCAGG
CACACCGGCCAGCACCACCCACAGCCCAAATTCCTAAAGAAATATTTGGGTCCCAG
CTTGGCCCGAGTCTCTGTTGTCCTGGGGAAGGACATCAAGATCTGAGTGTATGATGG
CCTGGGGCCTTGCATGTGGTGGGGGTCCAAGCCTGCCTCTGCTCACTTGTTCTGCAG
ACTGGCATGTTCTCTGTGATACTTACATACTTGTTTAACACTTCAGGGAAGAAAAGT
CAGAAGACCAGGACCTCCAGGGCCTCAAGGACAAACCCCTCAAGTTTAAAAAGGTG
AAGAAAGATAAGAAAGAAGAGAAAGAGGGCAAGCATGAGCCCGTGCAGCCATCAG
CCCACCACTCTGCTGAGCCCGCAGAGGCAGGCAAAGCAGAGACATCAGAAGGGTCA
GGCTCCGCCCCGGCTGTGCCGGAAGCTTCTGCCTCCCCCAAACAGCGGCGCTCCATC
ATCCGTGACCGGGGACCCATGTATGATGACCCCACCCTGCCTGAAGGCTGGACATG
GAAGCTTAAGCAAAGGAAATCTGGCCGCTCTGCTGGGAAGTATGATGTGTATTTGAT
CAAGTAAGTAAGAGCAACTCCTATCTCTACAGGGCAGGGAGGGCAGGGACAAGGAT
CCCTCATGGAGCAGGAAAATGTATGTGCCCAGGGTGGGGTCGGGGGGAACATAAAC
AATGAACACTGAGACCAGGTGTGCTTGAAATGACCGTGTACAGAGGTCGCTGCCCT
GAGTGGGAAGTTCTCAAGGTAGCAGGCCCTCTATCCTCTCCACACCTCAAGTCTTTA
TCTGGGGATGGAATAGCTGCGGAAGCAGAGGAACTTGCAGAGCCAGGGGTTCAGAG
GGGTGAAGAAGCATGTTTCAGTTCTGCCTTTTAAATGATCCCAAAAAGGTTAGCAGT
TTTCAAATGACATTTGCAGACAGCCTCATTTAATTCCATGAGAAGGGTGAGCAAAGG
ATTATCTTGTTGAAACTGATTCCTGGAGAGACTGAGCACACTAGTGACTACAAAGAC
CATGACGG
(C) Crossover pattern C (Clones 23, 35, 37)
(SEQ ID NO: 20)
GCTCACAGTAAGGATGCCTAGATGGGGTTACCTGTTGTCAAGATAAGGAAACTGAA
GCACAGAATGCTGAGGTCATTTGCTGGGTTCATGTTTGGAAAGCGGCAAAGGATTTC
AGTGCAGGTTGGCTGGCTCCAAACCTGTGTGTGCTTTCCATGACACTGTACTGTGTG
CCTCATTGAGCCTCATTCTAGAAAACCAAAAACACACCCAAGGCCCGGCCTTCACA
AAGGAGACCCCTCCCCCATTTGGCTCCCTTTCCAGCAGTCGACGGCCTCTTGTCAGC
CATCGAGCCCAGAGTCCCTTGAAGTGCGACTCATGCTGGGGTGGTATGCTCAGGAGC
CGCAGTGTTTCCGCTCAGAGGAAAGGGCTCTGATTCTCCTGCAGTGCTAGGAGACTT
GTGGGTGGCCACAGTGCAGGTCAGGCACACCGGCCAGCACCACCCACAGCCCAAAT
TCCTAAAGAAATATTTGGGTCCCAGCTTGGCCCGAGTCTCTGTTGTCCTGGGAGGTT
TCCCGGAACCTTTTGGAAAACCGGAAGGACATCAAGATCTGAGTGTATGATGGCCT
GGGGCCTTGCATGTGGTGGGGGTCCAAGCCTGCCTCTGCTCACTTGTTCTGCAGACT
GGCATGTTCTCTGTGATACTTACATACTTGTTTAACACTTCAGGGAAGAAAAGTCAG
AAGACCAGGACCTCCAGGGCCTCAAGGACAAACCCCTCAAGTTTAAAAAGGTGAAG
AAAGATAAGAAAGAAGAGAAAGAGGGCAAGCATGAGCCCGTGCAGCCATCAGCCC
ACCACTCTGCTGAGCCCGCAGAGGCAGGCAAAGCAGAGACATCAGAAGGGTCAGG
CTCCGCCCCGGCTGTGCCGGAAGCTTCTGCCTCCCCCAAACAGCGGCGCTCCATCAT
CCGTGACCGGGGACCCATGTATGATGACCCCACCCTGCCTGAAGGCTGGACACGGA
AGCTTAAGCAAAGGAAATCTGGCCGCTCTGCTGGGAAGTATGATGTGTATTTGATCA
AGTAAGTAAGAGCAACTCCTATCTCTACAGGGCAGGGAGGGCAGGGACAAGGATCC
CTCATGGAGCAGGAAAATGTATGTGCCCAGGGTGGGGTCGGGGGGAACATAAACAA
TGAACACTGAGACCAGGTGTGCTTGAAATGACCGTGTACAGAGGTCGCTGCCCTGA
GTGGGAAGTTCTCAAGGTAGCAGGCCCTCTATCCTCTCCACACCTCAAGTCTTTATCT
GGGGATGGAATAGCTGCGGAAGCAGAGGAACTTGCAGAGCCAGGGGTTCAGAGGG
GTGAAGAAGCATGTTTCAGTTCTGCCTTTTAAATGATCCCAAAAAGGTTAGCAGTTT
TCAAATGACATTTGCAGACAGCCTCATTTAATTCCATGAGAAGGGTGAGCAAAGGA
TTATCTTGTTGAAACTGATTCCTGGAGAGACTGAGCACACTAGTGACTACAAAGACC
ATGACGG
(D) Crossover pattern D (Clones 3, 10, 32)
(SEQ ID NO: 21)
GGGTTACCTGTTGTCAAGATAAGGAAACTGAAGCACAGAATGCTGAGGTCATTTGCT
GGGTTCATGTTTGGAAAGCGGCAAAGGATTTCAGTGCAGGTTGGCTGGCTCCAAACC
TGTGTGTGCTTTCCATGACACTGTACTGTGTGCCTCATTGAGCCTCATTCTAGAAAAC
CAAAAACACACCCAAGGCCCGGCCTTCACAAAGGAGACCCCTTCCCCATTTGGCTCC
CTTTCCAGCAGTCGACGGCCTCTTGTCAGCCATCGAGCCCAGAGTCCCTTGAAGTGC
GACTCATGCTGGGGTGGTATGCTCAGGAGCCGCAGTGTTTCCGCTCAGAGGAAAGG
GCTCTGATTCTCCTGCAGTGCTAGGAGACTTGTGGGTGGCCACAGTGCAGGTCAGGC
ACACCGGCCAGCACCACCCACAGCCCAAATTCCTAAAGAAATATTTGGGTCCCAGC
TTGGCCCGAGTCTCTGTTGTCCTGGGAGGTTTCCCGGAACCTTTTGGAAAACCGGAA
GGACATCAAGATCTGAGTGTATGATGGCCTGGGGCCTTGCATGTGGTGGGGGTCCA
AGCCTGCCTCTGCTCACTTGTTCTGCAGACTGGCATGTTCTCTGTGATACTTACATAC
TTGTTTAACACTTCAGGGAAGAAAAGTCAGAAGACCAGGACCTCCAGGGCCTCAAG
GACAAACCCCTCAAGTTTAAAAAGGTGAAGAAAGATAAGAAAGAAGAGAAAGAGG
GCAAGCATGAGCCCGTGCAGCCATCAGCCCACCACTCTGCTGAGCCCGCAGAGGCA
GGCAAAGCAGAGACATCAGAAGGGTCAGGCTCCGCCCCGGCTGTGCCGGAAGCTTC
TGCCTCCCCCAAACAGCGGCGCTCCATCATCCGTGACCGGGGACCCATGTATGATGA
CCCCACCCTGCCTGAAGGCTGGACACGGAAGCTTAAGCAAAGGAAATCTGGCCGCT
CTGCTGGGAAGTATGATGTGTATTTGATCAAGTAAGTAAGAGCAACTCCTATCTCTA
CAGGGCAGGGAGGGCAGGGACAAGGATCCCTCATGGAGCAGGAAAATGTATGTGC
CCAGGGTGGGGTCGGGGGGAACATAAACAATGAACACTGAGACCAGGTGTGCTTGA
AATGACCGTGTACAGAGGTCGCTGCCCTGAGTGGGAAGTTCTCAAGGTAGCAGGCC
CTCTATCCTCTCCACACCTCAAGTCTTTATCTGGGGATGGAATAGCTGCGGAAGCAG
AGGAACTTGCAGAGCCAGGGGTTCAGAGGGGTGAAGAAGCATGTTTCAGTTCTGCC
TTTTAAATGATCCCAAAAAGGTTAGCAGTTTTCAAATGACATTTGCAGACAGCCTCA
TTTAATTCCATGAGAAGGGTGAGCAAAGGATTATCTTGTTGAAACTGATTCCTGGAG
AGACTGAGCACACTAGTGACTACAAAGACCATGACGG

### Correction of S134C Mutation (Exon 4)

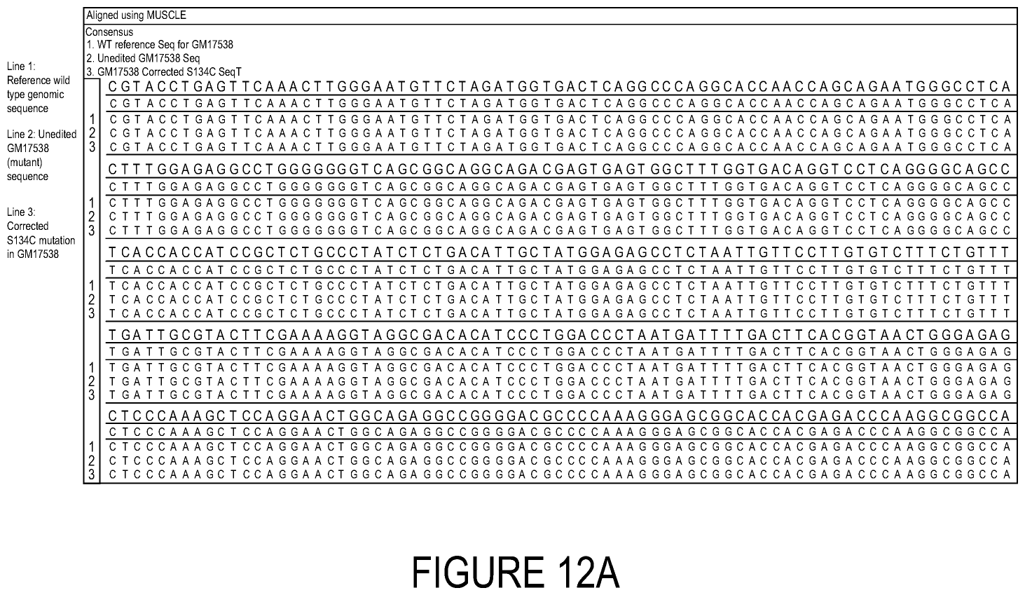

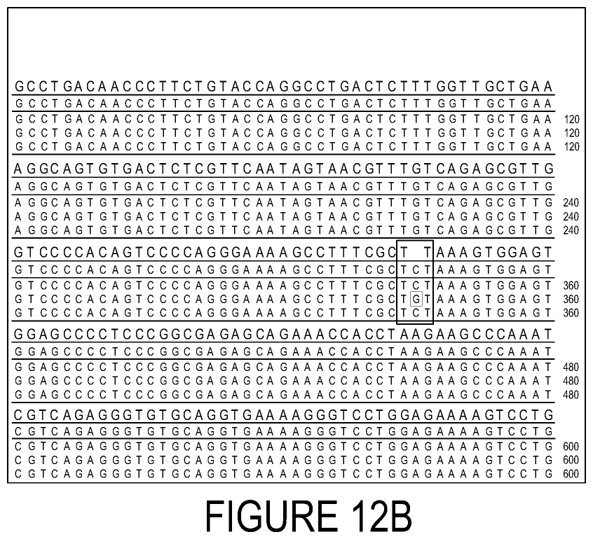

Sequences of a Region of the Wild Type Reference MECP2 Gene, the Unedited GM17538 Mutant MECP2 Gene and Edited (Corrected) MECP2 Gene in AAVHSC7-Transduced Male GM17538 Cells were Aligned (FIG. 12).

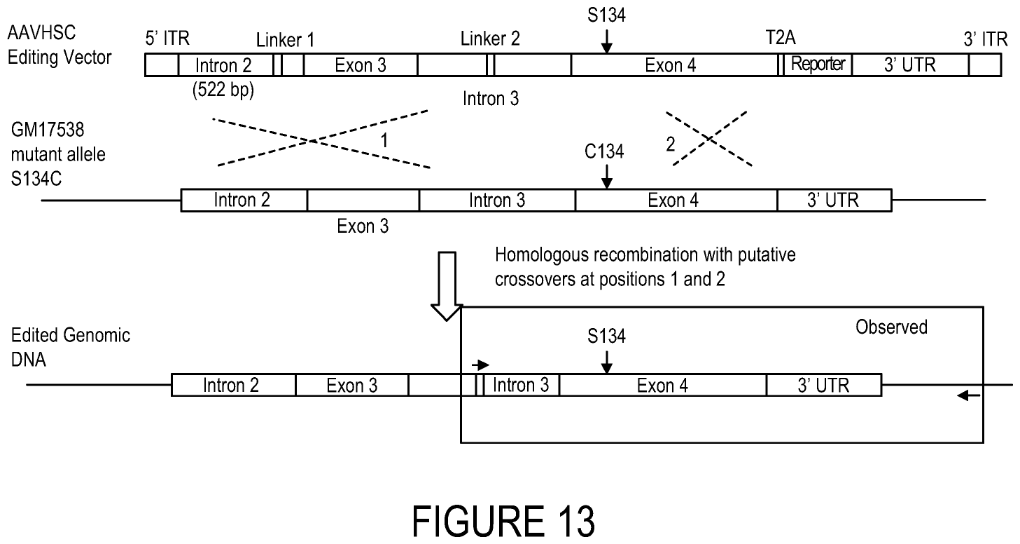

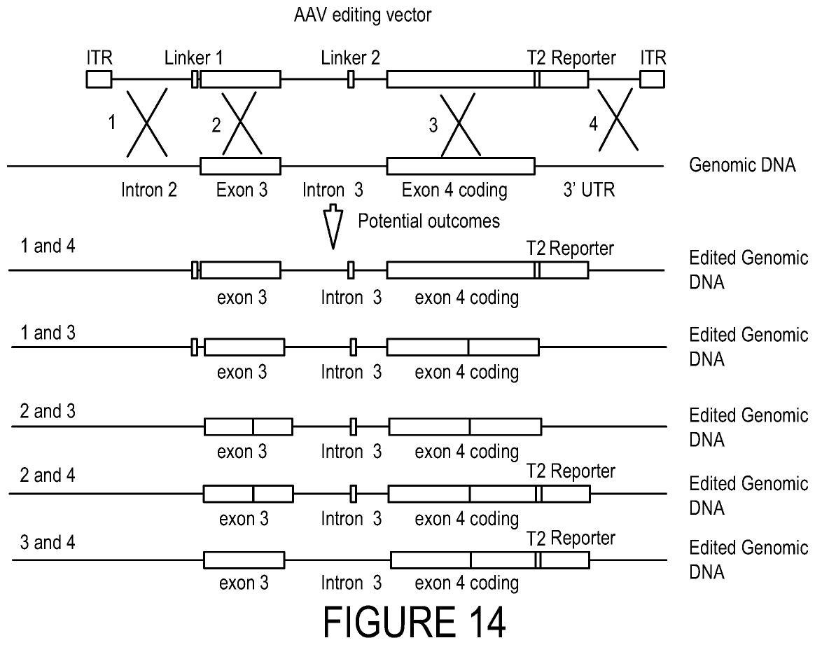

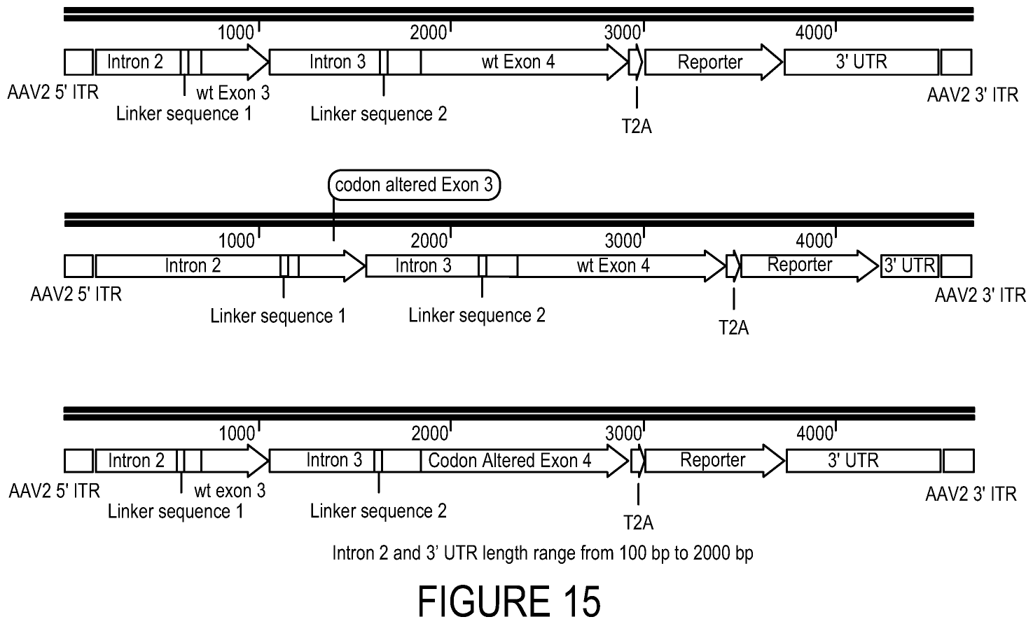

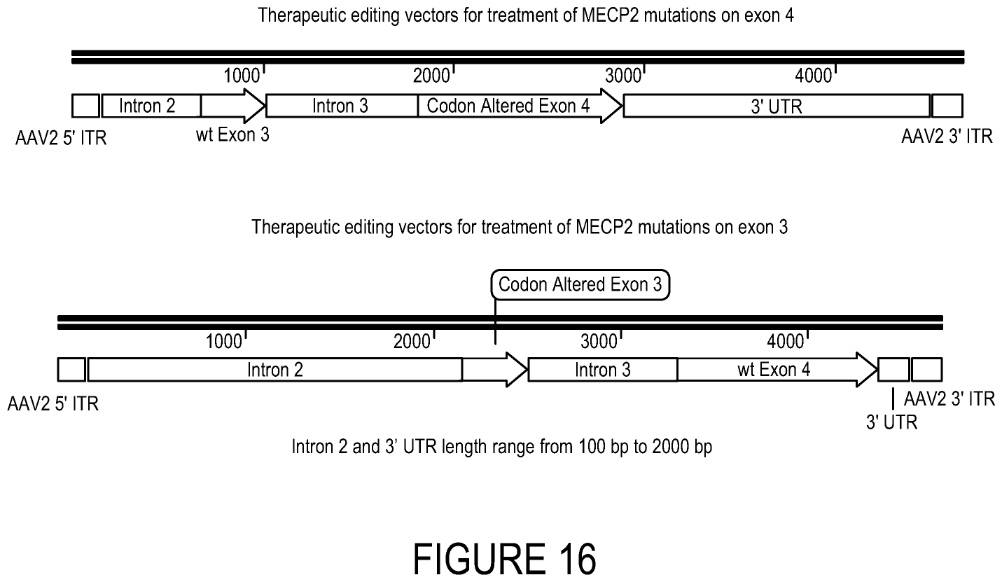

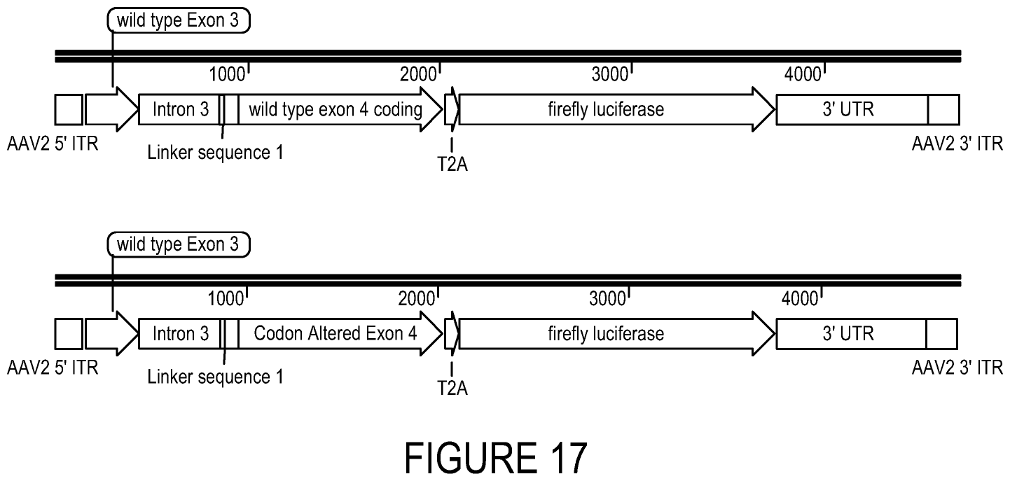

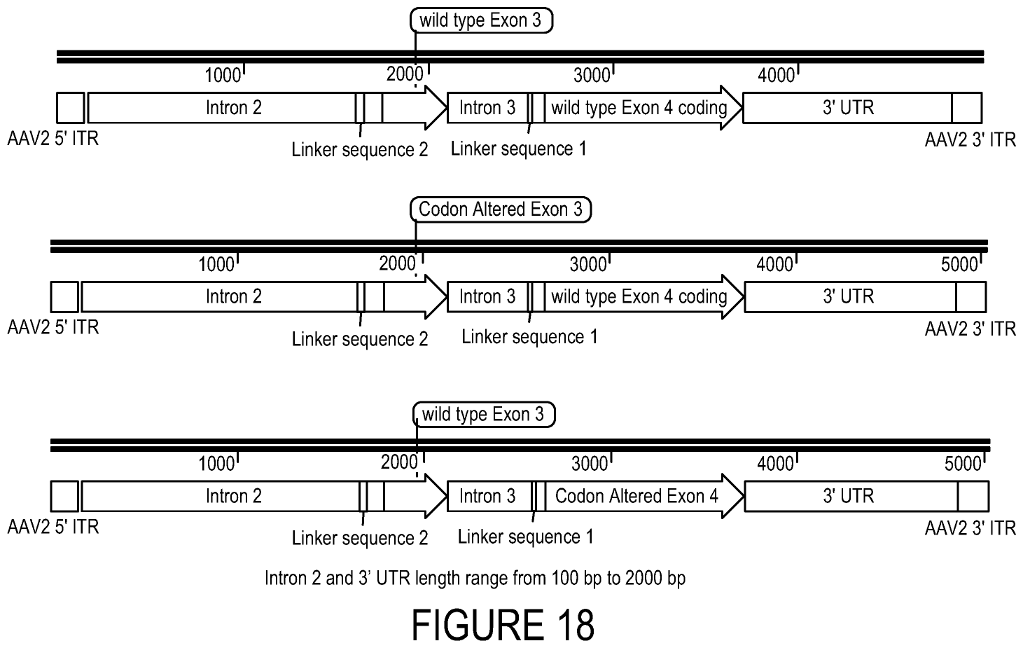

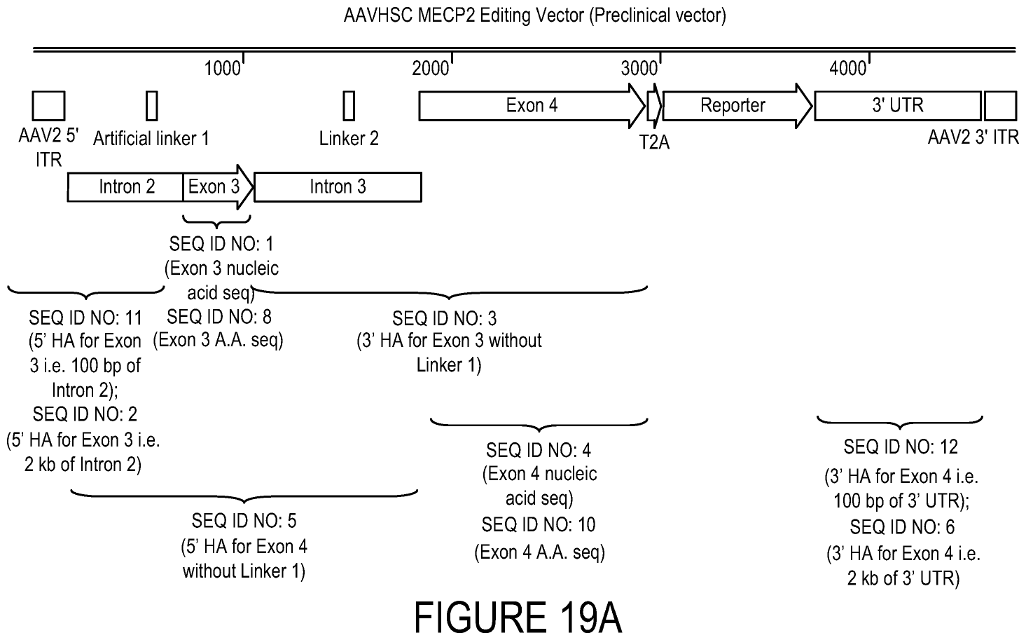

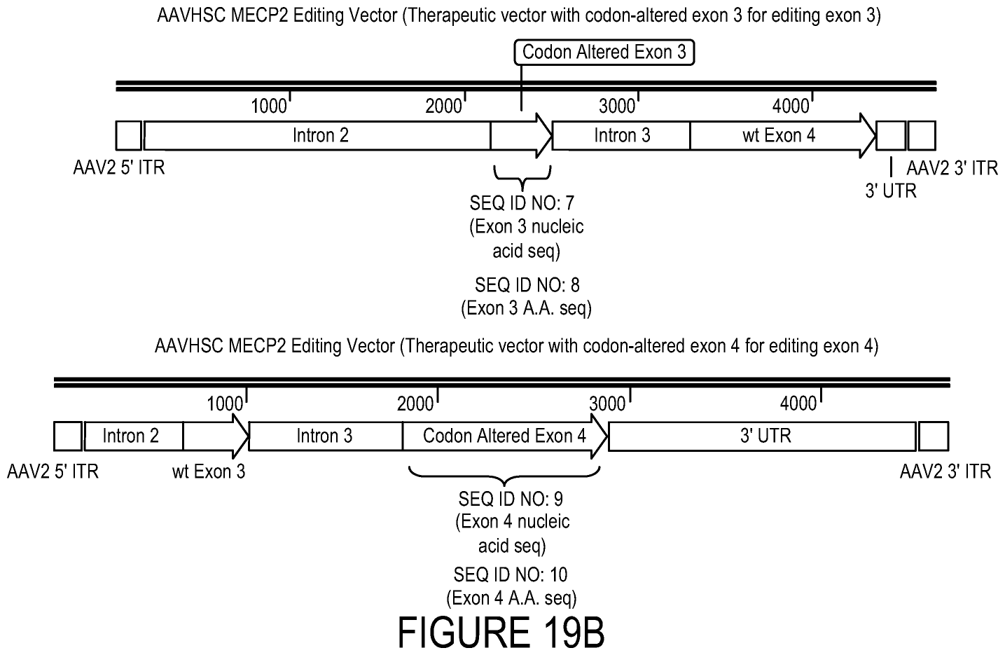

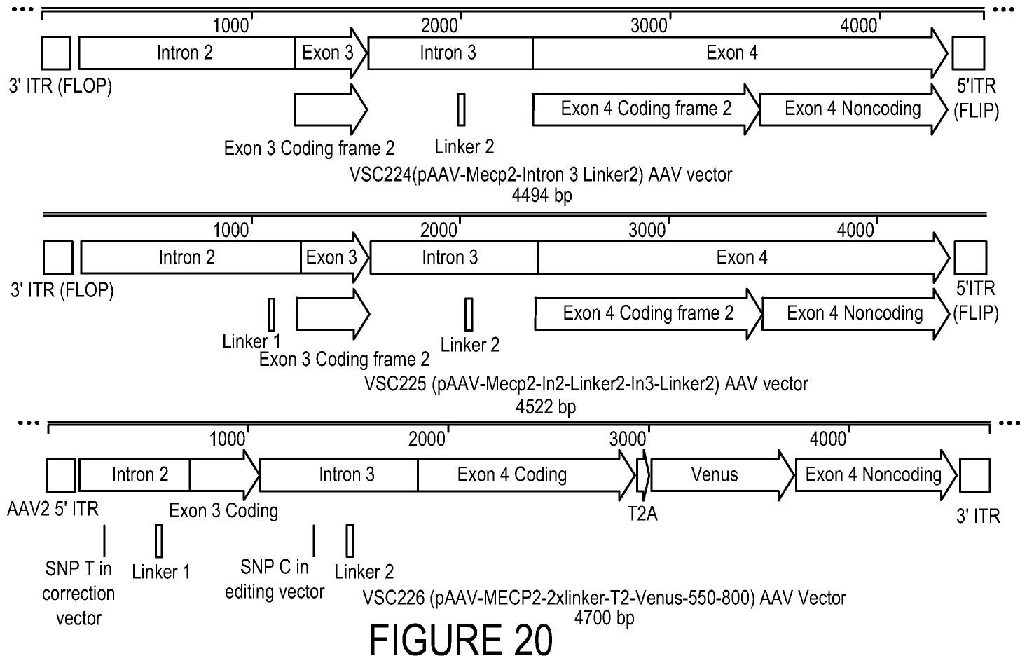

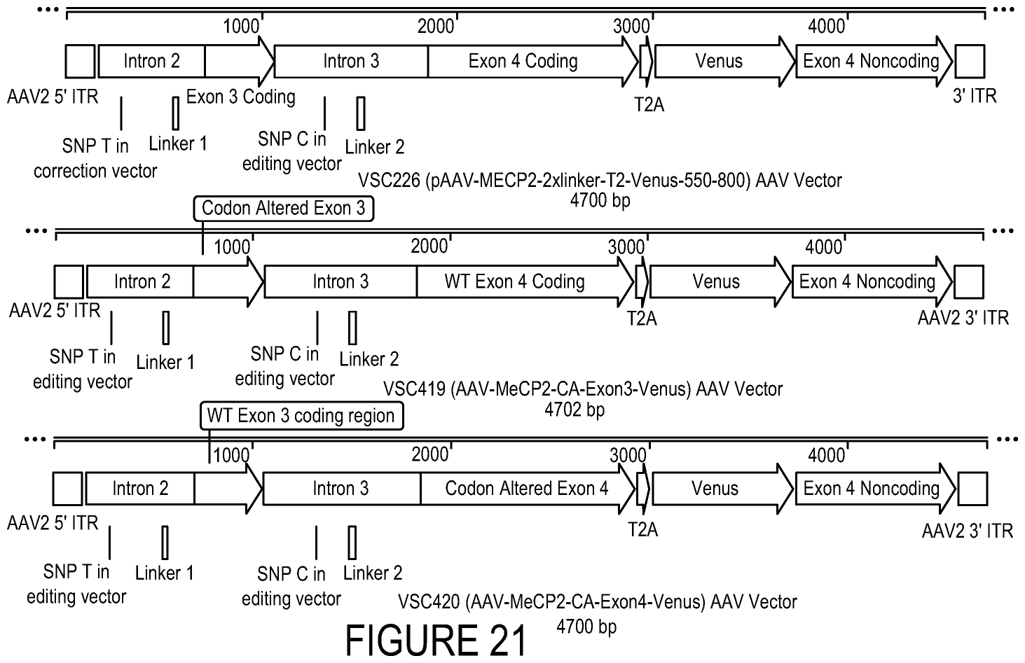

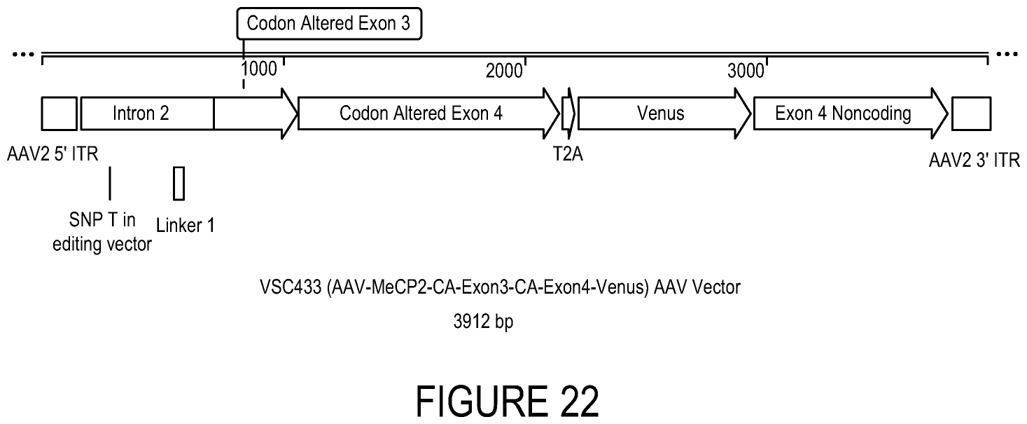

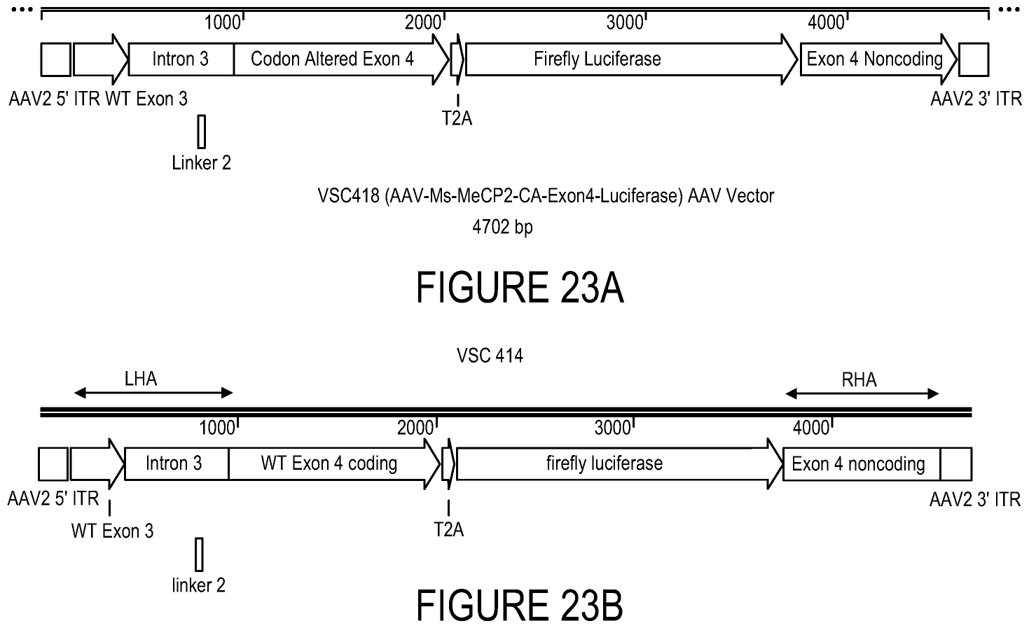

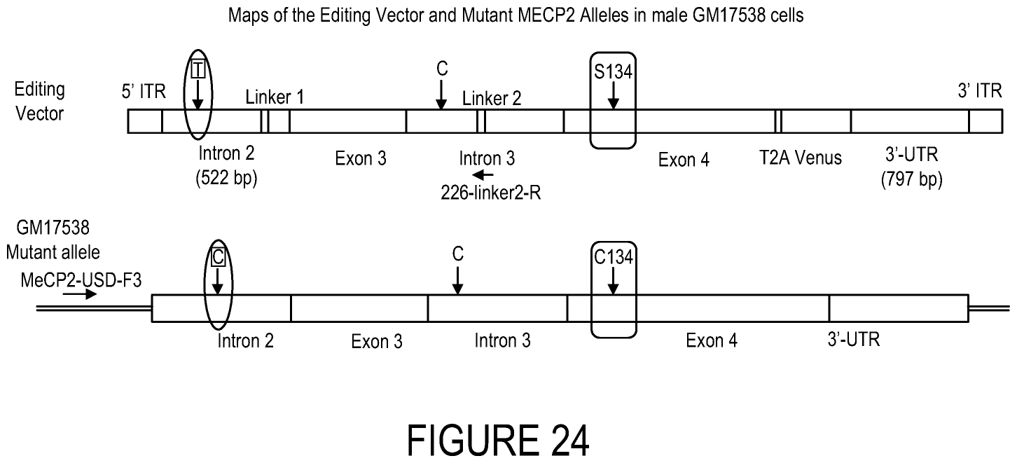

Sequences were amplified with primers linker2-F1 and MeCP2-DSD-R2. GM17538 cells were transduced with the AAVHSC7 MECP2 editing vector and after 48 h, the cells were harvested and genomic DNA was extracted. TI assays were then performed using a forward primer annealing to linker 2 and a genomic primer downstream of 3′ HA. The amplified product obtained was cloned and the clones were sequenced. The sequencing of clones revealed the correction of mutation at nucleotide 23 of exon 4, where the corrected codon TCT encodes for amino acid serine. The potential crossover pattern resulting in correction of mutation is represented in FIG. 13. The sequences of clones with correction of mutation are shown in the annotated sequence below.

Reference wild type MECP2 gene sequence (SEQ ID NO: 22):
CGTACCTGAGTTCAAACTTGGGAATGTTCTAGATGGTGACTCAGGCCCAGGCACCAA
CCAGCAGAATGGGCCTCAGCCTGACAACCCTTCTGTACCAGGCCTGACTCTTTGGTT
GCTGAACTTTGGAGAGGCCTGGGGGGGTCAGCGGCAGGCAGACGAGTGAGTGGCTT
TGGTGACAGGTCCTCAGGGGCAGCCAGGCAGTGTGACTCTCGTTCAATAGTAACGTT
TGTCAGAGCGTTGTCACCACCATCCGCTCTGCCCTATCTCTGACATTGCTATGGAGA
GCCTCTAATTGTTCCTTGTGTCTTTCTGTTTGTCCCCACAGTCCCCAGGGAAAAGCCT
TTCGCTCTAAAGTGGAGTTGATTGCGTACTTCGAAAAGGTAGGCGACACATCCCTGG
ACCCTAATGATTTTGACTTCACGGTAACTGGGAGAGGGAGCCCCTCCCGGCGAGAG
CAGAAACCACCTAAGAAGCCCAAATCTCCCAAAGCTCCAGGAACTGGCAGAGGCCG
GGGACGCCCCAAAGGGAGCGGCACCACGAGACCCAAGGCGGCCACGTCAGAGGGT
GTGCAGGTGAAAAGGGTCCTGGAGAAAAGTCCTGGGAAGCTCCTTGTCAAGATGCC
TTTTCAAACTTCGCCAGGGGGCAAGGCTGAGGGGGGTGGGGCCACCACATCCACCC
AGGTCATGGTGATCAAACGCCCCGGCAGGAAGCGAAAAGCTGAGGCCGACCCTCAG
GCCATTCCCAAGAAACGGGGCCGAAAGCCGGGGAGTGTGGTGGCAGCCGCTGCCGC
CGAGGCCAAAAAGAAAGCCGTGAAGGAGTCTTCTATCCGATCTGTGCAGGAGACCG
TACTCCCCATCAAGAAGCGCAAGACCCGGGAGACGGTCAGCATCGAGGTCAAGGAA
GTGGTGAAGCCCCTGCTGGTGTCCACCCTCGGTGAGAAGAGCGGGAAAGGACTGAA
GACCTGTAAGAGCCCTGGGCGGAAAAGCAAGGAGAGCAGCCCCAAGGGGCGCAGC
AGCAGCGCCTCCTCACCCCCCAAGAAGGAGCACCACCACCATCACCACCACTCAGA
GTCCCCAAAGGCCCCCGTGCCACTGCTCCCACCCCTGCCCCCACCTCCACCTGAGCC
CGAGAGCTCCGAGGACCCCACCAGCCCCCCTGAGCCCCAGGACTTGAGCAGCAGCG
TCTGCAAAGAGGAGAAGATGCCCAGAGGAGGCTCACTGGAGAGCGACGGCTGCCCC
AAGGAGCCAGCTAAGACTCAGCCCGCGGTTGCCACCGCCGCCACGGCCGCAGAAAA
GTACAAACACCGAGGGGAGGGAGAGCGCAAAGACATTGTTTCATCCTCCATGCCAA
GGCCAAACAGAGAGGAGCCTGTGGACAGCCGGACGCCCGTGACCGAGAGAGTTAG
CTGACTTTACACGGAGCGGATTGCAAAGCAAACCAACAAGAATAAAGGCAGCTGTT
GTCTCTTCTCCTTATGGGTAGGGCTCTGACAAAGCTTCCCGATTAACTGAAATAAAA
AATATTTTTTTTTCTTTCAGTAAACTTAGAGTTTCGTGGCTTCAGGGTGGGAGTAGTT
GGAGCATTGGGGATGTTTTTCTTACCGACAAGCACAGTCAGGTTGAAGACCTAACCA
GGGCCAGAAGTAGCTTTGCACTTTTCTAAACTAGGCTCCTTCAACAAGGCTTGCTGC
AGATACTACTGACCAGACAAGCTGTTGACCAGGCACCTCCCCTCCCGCCCAAACCTT
TCCCCCATGTGGTCGTTAGAGACAGAGCGACAGAGCAGTTGAGAGGACACTCCCGT
TTTCGGTGCCATCAGTGCCCCGTCTACAGCTCCCCCAGCTCCCCCCACCTCCCCCACT
CCCAACCACGTTGGGACAGGGAGGTGTGAGGCAGGAGAGACAGTTGGATTCTTTAG
AGAAGATGGATATGACCAGTGGCTATGGCCTGTGCGATCCCACCCGTGGTGGCTCA
AGTCTGGCCCCACACCAGCCCCAATCCAAAACTGGCAAGGACGCTTCACAGGACAG
GAAAGTGGCACCTGTCTGCTCCAGCTCTGGCATGGCTAGGAGGGGGGAGTCCCTTG
AACTACTGGGTGTAGACTGGCCTGAACCACAGGAGAGGATGGCCCAGGGTGAGGTG
GCATGGTCCATTCTCAAGGGACGTCCTCCAACGGGTGGCGCTAGAGGCCATGGAGG
CAGTAGGACAAGGTGCAGGCAGGCTGGCCTGGGGTCAGGCCGGGCAGAGCACAGC
GGGGTGAGAGGGATTCCTAATCACTCAGAGCAGTCTGTGACTTAGTGGACAGGGGA
GGGGGCAAAGGGGGAGGAGAAGAAAATGTTCTTCCAGTTACTTTCCAATTCTCCTTT
AGGGACAGCTTAGAATTATTTGCACTATTGAGTCTTCATGTTCCC
Unedited Mutant GM17538 (S134C) sequence (SEQ ID NO: 23):
CGTACCTGAGTTCAAACTTGGGAATGTTCTAGATGGTGACTCAGGCCCAGGCACCAA
CCAGCAGAATGGGCCTCAGCCTGACAACCCTTCTGTACCAGGCCTGACTCTTTGGTT
GCTGAACTTTGGAGAGGCCTGGGGGGGTCAGCGGCAGGCAGACGAGTGAGTGGCTT
TGGTGACAGGTCCTCAGGGGCAGCCAGGCAGTGTGACTCTCGTTCAATAGTAACGTT
TGTCAGAGCGTTGTCACCACCATCCGCTCTGCCCTATCTCTGACATTGCTATGGAGA
GCCTCTAATTGTTCCTTGTGTCTTTCTGTTTGTCCCCACAGTCCCCAGGGAAAAGCCT
TTCGCTGTAAAGTGGAGTTGATTGCGTACTTCGAAAAGGTAGGCGACACATCCCTGG
ACCCTAATGATTTTGACTTCACGGTAACTGGGAGAGGGAGCCCCTCCCGGCGAGAG
CAGAAACCACCTAAGAAGCCCAAATCTCCCAAAGCTCCAGGAACTGGCAGAGGCCG
GGGACGCCCCAAAGGGAGCGGCACCACGAGACCCAAGGCGGCCACGTCAGAGGGT
GTGCAGGTGAAAAGGGTCCTGGAGAAAAGTCCTGGGAAGCTCCTTGTCAAGATGCC
TTTTCAAACTTCGCCAGGGGGCAAGGCTGAGGGGGGTGGGGCCACCACATCCACCC
AGGTCATGGTGATCAAACGCCCCGGCAGGAAGCGAAAAGCTGAGGCCGACCCTCAG
GCCATTCCCAAGAAACGGGGCCGAAAGCCGGGGAGTGTGGTGGCAGCCGCTGCCGC
CGAGGCCAAAAAGAAAGCCGTGAAGGAGTCTTCTATCCGATCTGTGCAGGAGACCG
TACTCCCCATCAAGAAGCGCAAGACCCGGGAGACGGTCAGCATCGAGGTCAAGGAA
GTGGTGAAGCCCCTGCTGGTGTCCACCCTCGGTGAGAAGAGCGGGAAAGGACTGAA
GACCTGTAAGAGCCCTGGGCGGAAAAGCAAGGAGAGCAGCCCCAAGGGGCGCAGC
AGCAGCGCCTCCTCACCCCCCAAGAAGGAGCACCACCACCATCACCACCACTCAGA
GTCCCCAAAGGCCCCCGTGCCACTGCTCCCACCCCTGCCCCCACCTCCACCTGAGCC
CGAGAGCTCCGAGGACCCCACCAGCCCCCCTGAGCCCCAGGACTTGAGCAGCAGCG
TCTGCAAAGAGGAGAAGATGCCCAGAGGAGGCTCACTGGAGAGCGACGGCTGCCCC
AAGGAGCCAGCTAAGACTCAGCCCGCGGTTGCCACCGCCGCCACGGCCGCAGAAAA
GTACAAACACCGAGGGGAGGGAGAGCGCAAAGACATTGTTTCATCCTCCATGCCAA
GGCCAAACAGAGAGGAGCCTGTGGACAGCCGGACGCCCGTGACCGAGAGAGTTAG
CTGACTTTACACGGAGCGGATTGCAAAGCAAACCAACAAGAATAAAGGCAGCTGTT
GTCTCTTCTCCTTATGGGTAGGGCTCTGACAAAGCTTCCCGATTAACTGAAATAAAA
AATATTTTTTTTTCTTTCAGTAAACTTAGAGTTTCGTGGCTTCAGGGTGGGAGTAGTT
GGAGCATTGGGGATGTTTTTCTTACCGACAAGCACAGTCAGGTTGAAGACCTAACCA
GGGCCAGAAGTAGCTTTGCACTTTTCTAAACTAGGCTCCTTCAACAAGGCTTGCTGC
AGATACTACTGACCAGACAAGCTGTTGACCAGGCACCTCCCCTCCCGCCCAAACCTT
TCCCCCATGTGGTCGTTAGAGACAGAGCGACAGAGCAGTTGAGAGGACACTCCCGT
TTTCGGTGCCATCAGTGCCCCGTCTACAGCTCCCCCAGCTCCCCCCACCTCCCCCACT
CCCAACCACGTTGGGACAGGGAGGTGTGAGGCAGGAGAGACAGTTGGATTCTTTAG
AGAAGATGGATATGACCAGTGGCTATGGCCTGTGCGATCCCACCCGTGGTGGCTCA
AGTCTGGCCCCACACCAGCCCCAATCCAAAACTGGCAAGGACGCTTCACAGGACAG
GAAAGTGGCACCTGTCTGCTCCAGCTCTGGCATGGCTAGGAGGGGGGAGTCCCTTG
AACTACTGGGTGTAGACTGGCCTGAACCACAGGAGAGGATGGCCCAGGGTGAGGTG
GCATGGTCCATTCTCAAGGGACGTCCTCCAACGGGTGGCGCTAGAGGCCATGGAGG
CAGTAGGACAAGGTGCAGGCAGGCTGGCCTGGGGTCAGGCCGGGCAGAGCACAGC
GGGGTGAGAGGGATTCCTAATCACTCAGAGCAGTCTGTGACTTAGTGGACAGGGGA
GGGGGCAAAGGGGGAGGAGAAGAAAATGTTCTTCCAGTTACTTTCCAATTCTCCTTT
AGGGACAGCTTAGAATTATTTGCACTATTGAGTCTTCATGTTCCC
Edited (Corrected) GM17538 sequence with correction of mutation
(clones 6, 23) (SEQ ID NO: 24):
CAAAGACCATGACGGTATCGATCGTACCTGAGTTCAAACTTGGGAATGTTCTAGATGGTGA
CTCAGGCCCAGGCACCAACCAGCAGAATGGGCCTCAGCCTGACAACCCTTCTGTAC
CAGGCCTGACTCTTTGGTTGCTGAACTTTGGAGAGGCCTGGGGGGGTCAGCGGCAG
GCAGACGAGTGAGTGGCTTTGGTGACAGGTCCTCAGGGGCAGCCAGGCAGTGTGAC
TCTCGTTCAATAGTAACGTTTGTCAGAGCGTTGTCACCACCATCCGCTCTGCCCTATC
TCTGACATTGCTATGGAGAGCCTCTAATTGTTCCTTGTGTCTTTCTGTTTGTCCCCAC
AGTCCCCAGGGAAAAGCCTTTCGCTCTAAAGTGGAGTTGATTGCGTACTTCGAAAAG
GTAGGCGACACATCCCTGGACCCTAATGATTTTGACTTCACGGTAACTGGGAGAGGG
AGCCCCTCCCGGCGAGAGCAGAAACCACCTAAGAAGCCCAAATCTCCCAAAGCTCC
AGGAACTGGCAGAGGCCGGGGACGCCCCAAAGGGAGCGGCACCACGAGACCCAAG
GCGGCCACGTCAGAGGGTGTGCAGGTGAAAAGGGTCCTGGAGAAAAGTCCTGGGAA
GCTCCTTGTCAAGATGCCTTTTCAAACTTCGCCAGGGGGCAAGGCTGAGGGGGGTGG
GGCCACCACATCCACCCAGGTCATGGTGATCAAACGCCCCGGCAGGAAGCGAAAAG
CTGAGGCCGACCCTCAGGCCATTCCCAAGAAACGGGGCCGAAAGCCGGGGAGTGTG
GTGGCAGCCGCTGCCGCCGAGGCCAAAAAGAAAGCCGTGAAGGAGTCTTCTATCCG
ATCTGTGCAGGAGACCGTACTCCCCATCAAGAAGCGCAAGACCCGGGAGACGGTCA
GCATCGAGGTCAAGGAAGTGGTGAAGCCCCTGCTGGTGTCCACCCTCGGTGAGAAG
AGCGGGAAAGGACTGAAGACCTGTAAGAGCCCTGGGCGGAAAAGCAAGGAGAGCA
GCCCCAAGGGGCGCAGCAGCAGCGCCTCCTCACCCCCCAAGAAGGAGCACCACCAC
CATCACCACCACTCAGAGTCCCCAAAGGCCCCCGTGCCACTGCTCCCACCCCTGCCC
CCACCTCCACCTGAGCCCGAGAGCTCCGAGGACCCCACCAGCCCCCCTGAGCCCCA
GGACTTGAGCAGCAGCGTCTGCAAAGAGGAGAAGATGCCCAGAGGAGGCTCACTGG
AGAGCGACGGCTGCCCCAAGGAGCCAGCTAAGACTCAGCCCGCGGTTGCCACCGCC
GCCACGGCCGCAGAAAAGTACAAACACCGAGGGGAGGGAGAGCGCAAAGACATTG
TTTCATCCTCCATGCCAAGGCCAAACAGAGAGGAGCCTGTGGACAGCCGGACGCCC
GTGACCGAGAGAGTTAGCTGACTTTACACGGAGCGGATTGCAAAGCAAACCAACAA
GAATAAAGGCAGCTGTTGTCTCTTCTCCTTATGGGTAGGGCTCTGACAAAGCTTCCC
GATTAACTGAAATAAAAAATATTTTTTTTTCTTTCAGTAAACTTAGAGTTTCGTGGCT
TCAGGGTGGGAGTAGTTGGAGCATTGGGGATGTTTTTCTTACCGACAAGCACAGTCA
GGTTGAAGACCTAACCAGGGCCAGAAGTAGCTTTGCACTTTTCTAAACTAGGCTCCT
TCAACAAGGCTTGCTGCAGATACTACTGACCAGACAAGCTGTTGACCAGGCACCTCC
CCTCCCGCCCAAACCTTTCCCCCATGTGGTCGTTAGAGACAGAGCGACAGAGCAGTT
GAGAGGACACTCCCGTTTTCGGTGCCATCAGTGCCCCGTCTACAGCTCCCCCAGCTC
CCCCCACCTCCCCCACTCCCAACCACGTTGGGACAGGGAGGTGTGAGGCAGGAGAG
ACAGTTGGATTCTTTAGAGAAGATGGATATGACCAGTGGCTATGGCCTGTGCGATCC
CACCCGTGGTGGCTCAAGTCTGGCCCCACACCAGCCCCAATCCAAAACTGGCAAGG
ACGCTTCACAGGACAGGAAAGTGGCACCTGTCTGCTCCAGCTCTGGCATGGCTAGG
AGGGGGGAGTCCCTTGAACTACTGGGTGTAGACTGGCCTGAACCACAGGAGAGGAT
GGCCCAGGGTGAGGTGGCATGGTCCATTCTCAAGGGACGTCCTCCAACGGGTGGCG
CTAGAGGCCATGGAGGCAGTAGGACAAGGTGCAGGCAGGCTGGCCTGGGGTCAGGC
CGGGCAGAGCACAGCGGGGTGAGAGGGATTCCTAATCACTCAGAGCAGTCTGTGAC
TTAGTGGACAGGGGAGGGGGCAAAGGGGGAGGAGAAGAAAATGTTCTTCCAGTTAC
TTTCCAATTCTCCTTTAGGGACAGCTTAGAATTATTTGCACTATTGAGTCTTCATGTT
CCC

Sequence analysis for the R106W mutation revealed that the MECP2 gene had been successfully edited by the AAVHSC editing vector. Four distinct recombination patterns were observed over the region of homology between the editing vector and the MECP2 gene. The major patterns of recombination (Patterns A to D) were identified using the following markers: i. the SNP in intron 2; ii. Linker sequence 1; iii. the mutation at amino acid 106; iv. the SNP in intron 3; and v. the Linker sequence 2 (FIG. 11). FIG. 11 details the potential crossover events that may have generated the combinations of markers observed. Analysis of the sequences of the 17 TI clones showed that the sequence started in the chromosome external to the region of homology with the vector, demonstrating that the sequences represented the edited MECP2 gene and contained the 5′ junctions. No insertion/deletion mutations of AAV inverted terminal repeat sequences were observed in the edited MECP2 gene.

Sequence analysis revealed that 70% of edited alleles showed the correct (wild type) nucleotides at residue 106, indicating the feasibility of correcting pathogenic mutations associated with Rett Syndrome. Sequence analysis of the 17 clones indicated that the frequency of recombination increased with increasing distance from the ends of the region of homology. Using this information, we plan to design overlapping editing vectors to tile across exons 3 and 4 so that the pathogenic mutations are centrally located and therefore, be corrected at higher efficiency.

These data show a successful correction in male cells bearing S134C mutation, indicating that MECP2 gene on X chromosome was edited. In conclusion, the data obtained in our group demonstrate that AAVHSC vectors successfully and accurately edit the MECP2 gene without inducing mutations.

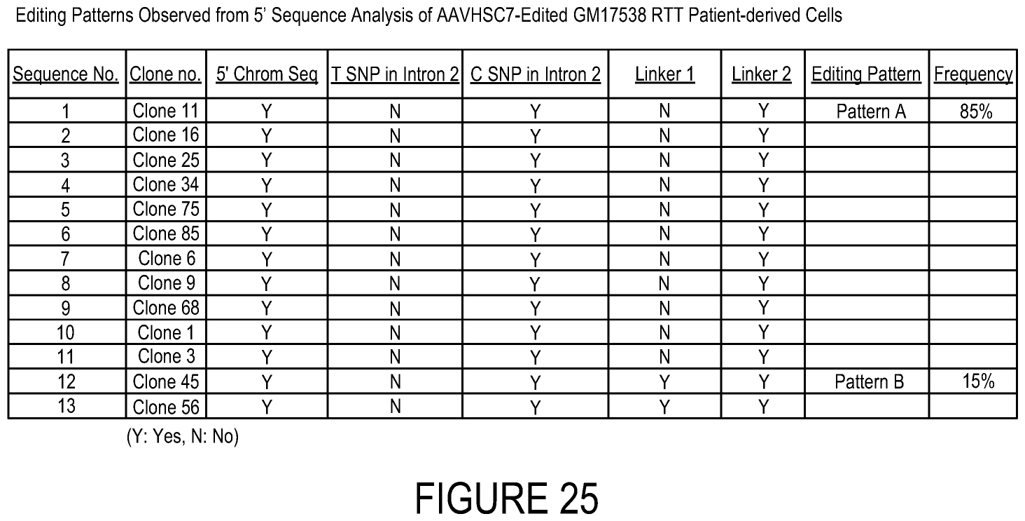

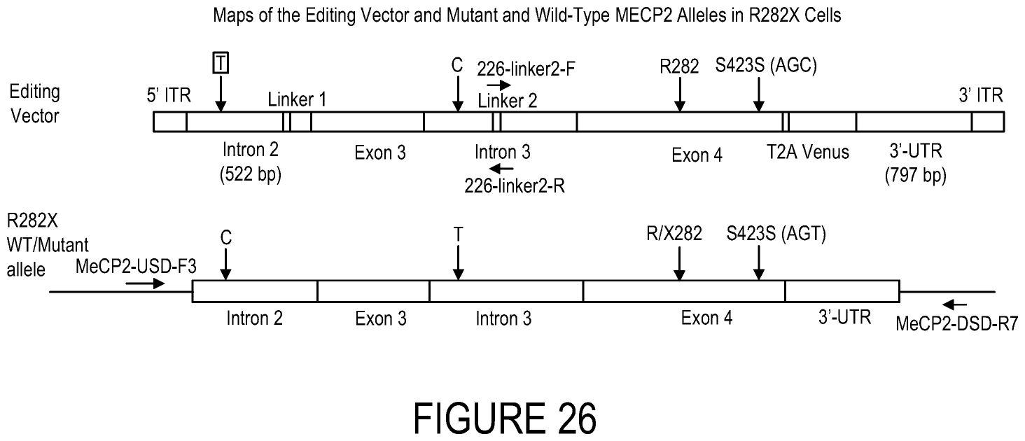

Editing Patterns Observed from 5′ Sequence Analysis of AAVHSC7-Edited GM17538 RTT Patient-Derived Cells (FIG. 25).

(A) Recombination Pattern A sequence
(SEQ ID NO: 35)
GGGTTACCTGTTGTCAAGATAAGGAAACTGAAGCACAGAATGCTGAGGTCATTTGCT
GGGTTCATGTTTGGAAAGCGGCAAAGGATTTCAGTGCAGGTTGGCTGGCTCCAAACC
TGTGTGTGCTTTCCATGACACTGTACTGTGTGCCTCATTGAGCCTCATTCTAGAAAAC
CAAAAACACACCCAAGGCCCGGCCTTCACAAAGGAGACCCCTCCCCCATTTGGCTC
CCTTTCCAGCAGTCGACGGCCTCTTGTCAGCCATCGAGCCCAGAGTCCCTTGAAGTG
CGACTCATGCTGGGGTGGTATGCTCAGGAGCCGCAGTGTTTCCGCTCAGAGGAAAG
GGCTCTGATTCTCCTGCAGTGCTAGGAGACTTGTGGGTGGCCACAGTGCAGGTCAGG
CACACCGGCCAGCACCACCCACAGCCCAAATTCCTAAAGAAATATTTGGGTCCCAG
CTTGGCCCGAGTCTCTGTTGTCCTGGGGAAGGACATCAAGATCTGAGTGTATGATGG
CCTGGGGCCTTGCATGTGGTGGGGGTCCAAGCCTGCCTCTGCTCACTTGTTCTGCAG
ACTGGCATGTTCTCTGTGATACTTACATACTTGTTTAACACTTCAGGGAAGAAAAGT
CAGAAGACCAGGACCTCCAGGGCCTCAAGGACAAACCCCTCAAGTTTAAAAAGGTG
AAGAAAGATAAGAAAGAAGAGAAAGAGGGCAAGCATGAGCCCGTGCAGCCATCAG
CCCACCACTCTGCTGAGCCCGCAGAGGCAGGCAAAGCAGAGACATCAGAAGGGTCA
GGCTCCGCCCCGGCTGTGCCGGAAGCTTCTGCCTCCCCCAAACAGCGGCGCTCCATC
ATCCGTGACCGGGGACCCATGTATGATGACCCCACCCTGCCTGAAGGCTGGACACG
GAAGCTTAAGCAAAGGAAATCTGGCCGCTCTGCTGGGAAGTATGATGTGTATTTGAT
CAAGTAAGTAAGAGCAACTCCTATCTCTACAGGGCAGGGAGGGCAGGGACAAGGAT
CCCTCATGGAGCAGGAAAATGTATGTGCCCAGGGTGGGGTCGGGGGGAACATAAAC
AATGAACACTGAGACCAGGTGTGCTTGAAATGACCGTGTACAGAGGTCGCTGCCCT
GAGTGGGAAGTTCTCAAGGTAGCAGGCCCTCTATCCTCTCCACACCTCAAGTCTTTA
TCTGGGGATGGAATAGCTGCGGAAGCAGAGGAACTTGCAGAGCCAGGGGTTCAGAG
GGGTGAAGAAGCATGTTTCAGTTCTGCCTTTTAAATGATCCCAAAAAGGTTAGCAGT
TTTCAAATGACATTTGCAGACAGCCTCATTTAATTCCATGAGAAGGGTGAGCAAAGG
ATTATCTTGTTGAAACTGATTCCTGGAGAGACTGAGCACACTAGTGACTACAAAGAC
CATGACGG
(B) Recombination Pattern B sequence
(SEQ ID NO: 36)
GGGTTACCTGTTGTCAAGATAAGGAAACTGAAGCACAGAATGCTGAGGTCATTTGCT
GGGTTCATGTTTGGAAAGCGGCAAAGGATTTCAGTGCAGGTTGGCTGGCTCCAAACC
TGTGTGTGCTTTCCATGACACTGTACTGTGTGCCTCATTGAGCCTCATTCTAGAAAAC
CAAAAACACACCCAAGGCCCGGCCTTCACAAAGGAGACCCCTCCCCCATTTGGCTC
CCTTTCCAGCAGTCGACGGCCTCTTGTCAGCCATCGAGCCCAGAGTCCCTTGAAGTG
CGACTCATGCTGGGGTGGTATGCTCAGGAGCCGCAGTGTTTCCGCTCAGAGGAAAG
GGCTCTGATTCTCCTGCAGTGCTAGGAGACTTGTGGGTGGCCACAGTGCAGGTCAGG
CACACCGGCCAGCACCACCCACAGCCCAAATTCCTAAAGAAATATTTGGGTCCCAG
CTTGGCCCGAGTCTCTGTTGTCCTGGGAGGTTTCCCGGAACCTTTTGGAAAACCGGA
AGGACATCAAGATCTGAGTGTATGATGGCCTGGGGCCTTGCATGTGGTGGGGGTCC
AAGCCTGCCTCTGCTCACTTGTTCTGCAGACTGGCATGTTCTCTGTGATACTTACATA
CTTGTTTAACACTTCAGGGAAGAAAAGTCAGAAGACCAGGACCTCCAGGGCCTCAA
GGACAAACCCCTCAAGTTTAAAAAGGTGAAGAAAGATAAGAAAGAAGAGAAAGAG
GGCAAGCATGAGCCCGTGCAGCCATCAGCCCACCACTCTGCTGAGCCCGCAGAGGC
AGGCAAAGCAGAGACATCAGAAGGGTCAGGCTCCGCCCCGGCTGTGCCGGAAGCTT
CTGCCTCCCCCAAACAGCGGCGCTCCATCATCCGTGACCGGGGACCCATGTATGATG
ACCCCACCCTGCCTGAAGGCTGGACACGGAAGCTTAAGCAAAGGAAATCTGGCCGC
TCTGCTGGGAAGTATGATGTGTATTTGATCAAGTAAGTAAGAGCAACTCCTATCTCT
ACAGGGCAGGGAGGGCAGGGACAAGGATCCCTCATGGAGCAGGAAAATGTATGTG
CCCAGGGTGGGGTCGGGGGGAACATAAACAATGAACACTGAGACCAGGTGTGCTTG
AAATGACCGTGTACAGAGGTCGCTGCCCTGAGTGGGAAGTTCTCAAGGTAGCAGGC
CCTCTATCCTCTCCACACCTCAAGTCTTTATCTGGGGATGGAATAGCTGCGGAAGCA
GAGGAACTTGCAGAGCCAGGGGTTCAGAGGGGTGAAGAAGCATGTTTCAGTTCTGC
CTTTTAAATGATCCCAAAAAGGTTAGCAGTTTTCAAATGACATTTGCAGACAGCCTC
ATTTAATTCCATGAGAAGGGTGAGCAAAGGATTATCTTGTTGAAACTGATTCCTGGA
GAGACTGAGCACACTAGTGACTACAAAGACCATGACGG

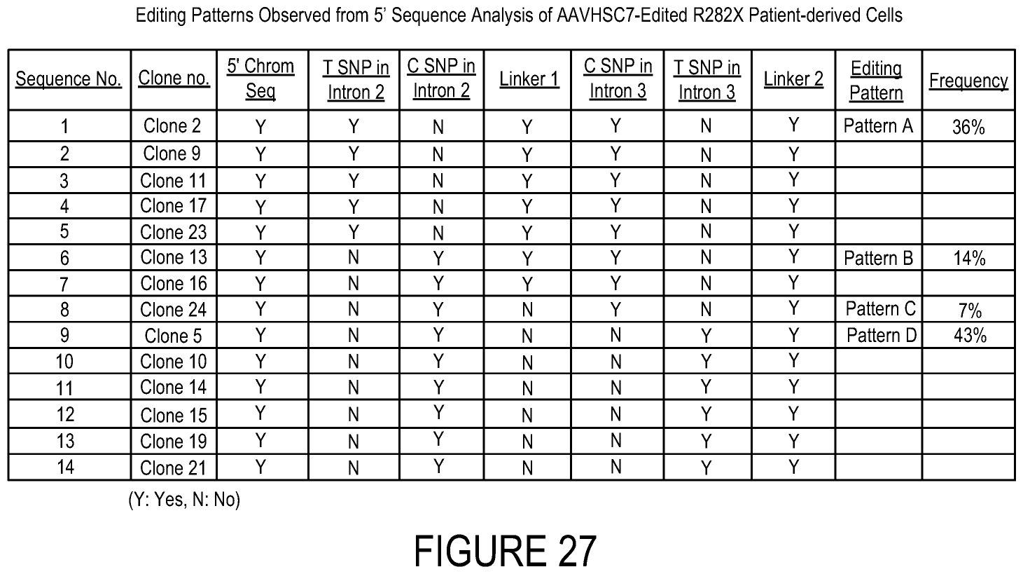

Editing Patterns Observed from 5′ Sequence Analysis of AAVHSC7-Edited R282X Patient-Derived Cells (FIG. 27)

(A) Recombination Pattern A sequence
(SEQ ID NO: 37)
GGGTTACCTGTTGTCAAGATAAGGAAACTGAAGCACAGAATGCTGAGGTCATTTGCT
GGGTTCATGTTTGGAAAGCGGCAAAGGATTTCAGTGCAGGTTGGCTGGCTCCAAACC
TGTGTGTGCTTTCCATGACACTGTACTGTGTGCCTCATTGAGCCTCATTCTAGAAAAC
CAAAAACACACCCAAGGCCCGGCCTTCACAAAGGAGACCCCTTCCCCATTTGGCTCC
CTTTCCAGCAGTCGACGGCCTCTTGTCAGCCATCGAGCCCAGAGTCCCTTGAAGTGC
GACTCATGCTGGGGTGGTATGCTCAGGAGCCGCAGTGTTTCCGCTCAGAGGAAAGG
GCTCTGATTCTCCTGCAGTGCTAGGAGACTTGTGGGTGGCCACAGTGCAGGTCAGGC
ACACCGGCCAGCACCACCCACAGCCCAAATTCCTAAAGAAATATTTGGGTCCCAGC
TTGGCCCGAGTCTCTGTTGTCCTGGGAGGTTTCCCGGAACCTTTTGGAAAACCGGAA
GGACATCAAGATCTGAGTGTATGATGGCCTGGGGCCTTGCATGTGGTGGGGGTCCA
AGCCTGCCTCTGCTCACTTGTTCTGCAGACTGGCATGTTCTCTGTGATACTTACATAC
TTGTTTAACACTTCAGGGAAGAAAAGTCAGAAGACCAGGACCTCCAGGGCCTCAAG
GACAAACCCCTCAAGTTTAAAAAGGTGAAGAAAGATAAGAAAGAAGAGAAAGAGG
GCAAGCATGAGCCCGTGCAGCCATCAGCCCACCACTCTGCTGAGCCCGCAGAGGCA
GGCAAAGCAGAGACATCAGAAGGGTCAGGCTCCGCCCCGGCTGTGCCGGAAGCTTC
TGCCTCCCCCAAACAGCGGCGCTCCATCATCCGTGACCGGGGACCCATGTATGATGA
CCCCACCCTGCCTGAAGGCTGGACACGGAAGCTTAAGCAAAGGAAATCTGGCCGCT
CTGCTGGGAAGTATGATGTGTATTTGATCAAGTAAGTAAGAGCAACTCCTATCTCTA
CAGGGCAGGGAGGGCAGGGACAAGGATCCCTCATGGAGCAGGAAAATGTATGTGC
CCAGGGTGGGGTCGGGGGGAACATAAACAATGAACACTGAGACCAGGTGTGCTTGA
AATGACCGTGTACAGAGGTCGCTGCCCTGAGTGGGAAGTTCTCAAGGTAGCAGGCC
CTCTATCCTCTCCACACCTCAAGTCTTTATCTGGGGATGGAATAGCTGCGGAAGCAG
AGGAACTTGCAGAGCCAGGGGTTCAGAGGGGTGAAGAAGCATGTTTCAGTTCTGCC
TTTTAAATGATCCCAAAAAGGTTAGCAGTTTTCAAATGACATTTGCAGACAGCCTCA
TTTAATTCCATGAGAAGGGTGAGCAAAGGATTATCTTGTTGAAACTGATTCCTGGAG
AGACTGAGCACACTAGTGACTACAAAGACCATGACGG
(B) Recombination Pattern B sequence
(SEQ ID NO: 38)
GGGTTACCTGTTGTCAAGATAAGGAAACTGAAGCACAGAATGCTGAGGTCATTTGCT
GGGTTCATGTTTGGAAAGCGGCAAAGGATTTCAGTGCAGGTTGGCTGGCTCCAAACC
TGTGTGTGCTTTCCATGACACTGTACTGTGTGCCTCATTGAGCCTCATTCTAGAAAAC
CAAAAACACACCCAAGGCCCGGCCTTCACAAAGGAGACCCCTCCCCCATTTGGCTC
CCTTTCCAGCAGTCGACGGCCTCTTGTCAGCCATCGAGCCCAGAGTCCCTTGAAGTG
CGACTCATGCTGGGGTGGTATGCTCAGGAGCCGCAGTGTTTCCGCTCAGAGGAAAG
GGCTCTGATTCTCCTGCAGTGCTAGGAGACTTGTGGGTGGCCACAGTGCAGGTCAGG
CACACCGGCCAGCACCACCCACAGCCCAAATTCCTAAAGAAATATTTGGGTCCCAG
CTTGGCCCGAGTCTCTGTTGTCCTGGGAGGTTTCCCGGAACCTTTTGGAAAACCGGA
AGGACATCAAGATCTGAGTGTATGATGGCCTGGGGCCTTGCATGTGGTGGGGGTCC
AAGCCTGCCTCTGCTCACTTGTTCTGCAGACTGGCATGTTCTCTGTGATACTTACATA
CTTGTTTAACACTTCAGGGAAGAAAAGTCAGAAGACCAGGACCTCCAGGGCCTCAA
GGACAAACCCCTCAAGTTTAAAAAGGTGAAGAAAGATAAGAAAGAAGAGAAAGAG
GGCAAGCATGAGCCCGTGCAGCCATCAGCCCACCACTCTGCTGAGCCCGCAGAGGC
AGGCAAAGCAGAGACATCAGAAGGGTCAGGCTCCGCCCCGGCTGTGCCGGAAGCTT
CTGCCTCCCCCAAACAGCGGCGCTCCATCATCCGTGACCGGGGACCCATGTATGATG
ACCCCACCCTGCCTGAAGGCTGGACACGGAAGCTTAAGCAAAGGAAATCTGGCCGC
TCTGCTGGGAAGTATGATGTGTATTTGATCAAGTAAGTAAGAGCAACTCCTATCTCT
ACAGGGCAGGGAGGGCAGGGACAAGGATCCCTCATGGAGCAGGAAAATGTATGTG
CCCAGGGTGGGGTCGGGGGGAACATAAACAATGAACACTGAGACCAGGTGTGCTTG
AAATGACCGTGTACAGAGGTCGCTGCCCTGAGTGGGAAGTTCTCAAGGTAGCAGGC
CCTCTATCCTCTCCACACCTCAAGTCTTTATCTGGGGATGGAATAGCTGCGGAAGCA
GAGGAACTTGCAGAGCCAGGGGTTCAGAGGGGTGAAGAAGCATGTTTCAGTTCTGC
CTTTTAAATGATCCCAAAAAGGTTAGCAGTTTTCAAATGACATTTGCAGACAGCCTC
ATTTAATTCCATGAGAAGGGTGAGCAAAGGATTATCTTGTTGAAACTGATTCCTGGA
GAGACTGAGCACACTAGTGACTACAAAGACCATGACGG
(C) Recombination Pattern C sequence
(SEQ ID NO: 39)
GGGTTACCTGTTGTCAAGATAAGGAAACTGAAGCACAGAATGCTGAGGTCATTTGCT
GGGTTCATGTTTGGAAAGCGGCAAAGGATTTCAGTGCAGGTTGGCTGGCTCCAAACC
TGTGTGTGCTTTCCATGACACTGTACTGTGTGCCTCATTGAGCCTCATTCTAGAAAAC
CAAAAACACACCCAAGGCCCGGCCTTCACAAAGGAGACCCCTCCCCCATTTGGCTC
CCTTTCCAGCAGTCGACGGCCTCTTGTCAGCCATCGAGCCCAGAGTCCCTTGAAGTG
CGACTCATGCTGGGGTGGTATGCTCAGGAGCCGCAGTGTTTCCGCTCAGAGGAAAG
GGCTCTGATTCTCCTGCAGTGCTAGGAGACTTGTGGGTGGCCACAGTGCAGGTCAGG
CACACCGGCCAGCACCACCCACAGCCCAAATTCCTAAAGAAATATTTGGGTCCCAG
CTTGGCCCGAGTCTCTGTTGTCCTGGGGAAGGACATCAAGATCTGAGTGTATGATGG
CCTGGGGCCTTGCATGTGGTGGGGGTCCAAGCCTGCCTCTGCTCACTTGTTCTGCAG
ACTGGCATGTTCTCTGTGATACTTACATACTTGTTTAACACTTCAGGGAAGAAAAGT
CAGAAGACCAGGACCTCCAGGGCCTCAAGGACAAACCCCTCAAGTTTAAAAAGGTG
AAGAAAGATAAGAAAGAAGAGAAAGAGGGCAAGCATGAGCCCGTGCAGCCATCAG
CCCACCACTCTGCTGAGCCCGCAGAGGCAGGCAAAGCAGAGACATCAGAAGGGTCA
GGCTCCGCCCCGGCTGTGCCGGAAGCTTCTGCCTCCCCCAAACAGCGGCGCTCCATC
ATCCGTGACCGGGGACCCATGTATGATGACCCCACCCTGCCTGAAGGCTGGACACG
GAAGCTTAAGCAAAGGAAATCTGGCCGCTCTGCTGGGAAGTATGATGTGTATTTGAT
CAAGTAAGTAAGAGCAACTCCTATCTCTACAGGGCAGGGAGGGCAGGGACAAGGAT
CCCTCATGGAGCAGGAAAATGTATGTGCCCAGGGTGGGGTCGGGGGGAACATAAAC
AATGAACACTGAGACCAGGTGTGCTTGAAATGACCGTGTACAGAGGTCGCTGCCCT
GAGTGGGAAGTTCTCAAGGTAGCAGGCCCTCTATCCTCTCCACACCTCAAGTCTTTA
TCTGGGGATGGAATAGCTGCGGAAGCAGAGGAACTTGCAGAGCCAGGGGTTCAGAG
GGGTGAAGAAGCATGTTTCAGTTCTGCCTTTTAAATGATCCCAAAAAGGTTAGCAGT
TTTCAAATGACATTTGCAGACAGCCTCATTTAATTCCATGAGAAGGGTGAGCAAAGG
ATTATCTTGTTGAAACTGATTCCTGGAGAGACTGAGCACACTAGTGACTACAAAGAC
CATGACGG
(D) Recombination Pattern D sequence
(SEQ ID NO: 40)
GGGTTACCTGTTGTCAAGATAAGGAAACTGAAGCACAGAATGCTGAGGTCATTTGCT
GGGTTCATGTTTGGAAAGCGGCAAAGGATTTCAGTGCAGGTTGGCTGGCTCCAAACC
TGTGTGTGCTTTCCATGACACTGTACTGTGTGCCTCATTGAGCCTCATTCTAGAAAAC
CAAAAACACACCCAAGGCCCGGCCTTCACAAAGGAGACCCCTCCCCCATTTGGCTC
CCTTTCCAGCAGTCGACGGCCTCTTGTCAGCCATCGAGCCCAGAGTCCCTTGAAGTG
CGACTCATGCTGGGGTGGTATGCTCAGGAGCCGCAGTGTTTCCGCTCAGAGGAAAG
GGCTCTGATTCTCCTGCAGTGCTAGGAGACTTGTGGGTGGCCACAGTGCAGGTCAGG
CACACCGGCCAGCACCACCCACAGCCCAAATTCCTAAAGAAATATTTGGGTCCCAG
CTTGGCCCGAGTCTCTGTTGTCCTGGGGAAGGACATCAAGATCTGAGTGTATGATGG
CCTGGGGCCTTGCATGTGGTGGGGGTCCAAGCCTGCCTCTGCTCACTTGTTCTGCAG
ACTGGCATGTTCTCTGTGATACTTACATACTTGTTTAACACTTCAGGGAAGAAAAGT
CAGAAGACCAGGACCTCCAGGGCCTCAAGGACAAACCCCTCAAGTTTAAAAAGGTG
AAGAAAGATAAGAAAGAAGAGAAAGAGGGCAAGCATGAGCCCGTGCAGCCATCAG
CCCACCACTCTGCTGAGCCCGCAGAGGCAGGCAAAGCAGAGACATCAGAAGGGTCA
GGCTCCGCCCCGGCTGTGCCGGAAGCTTCTGCCTCCCCCAAACAGCGGCGCTCCATC
ATCCGTGACCGGGGACCCATGTATGATGACCCCACCCTGCCTGAAGGCTGGACACG
GAAGCTTAAGCAAAGGAAATCTGGCCGCTCTGCTGGGAAGTATGATGTGTATTTGAT
CAAGTAAGTAAGAGCAACTCCTATCTCTACAGGGCAGGGAGGGCAGGGACAAGGAT
CCCTCATGGAGCAGGAAAATGTATGTGCCCAGGGTGGGGTCGGGGGGAACATAAAC
AATGAACACTGAGACCAGGTGTGCTTGAAATGACCGTGTACAGAGGTCGCTGCCCT
GAGTGGGAAGTTCTCAAGGTAGCAGGCCCTCTATCCTCTCCACACCTCAAGTCTTTA
TCTGGGGATGGAATAGCTGCGGAAGCAGAGGAACTTGCAGAGCTAGGGGTTCAGAG
GGGTGAAGAAGCATGTTTCAGTTCTGCCTTTTAAATGATCCCAAAAAGGTTAGCAGT
TTTCAAATGACATTTGCAGACAGCCTCATTTAATTCCATGAGAAGGGTGAGCAAAGG
ATTATCTTGTTGAAACTGATTCCTGGAGAGACTGAGCACACTAGTGACTACAAAGAC
CATGACGG

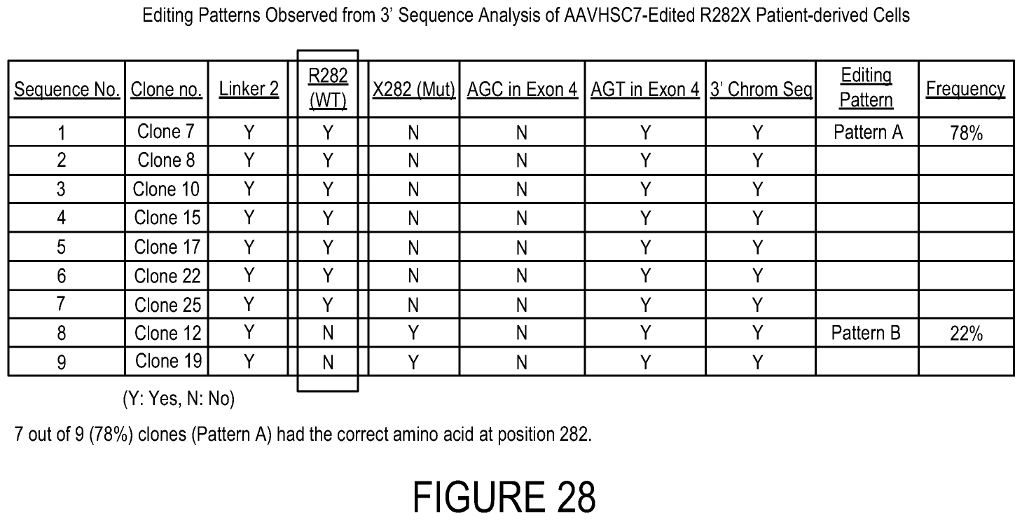

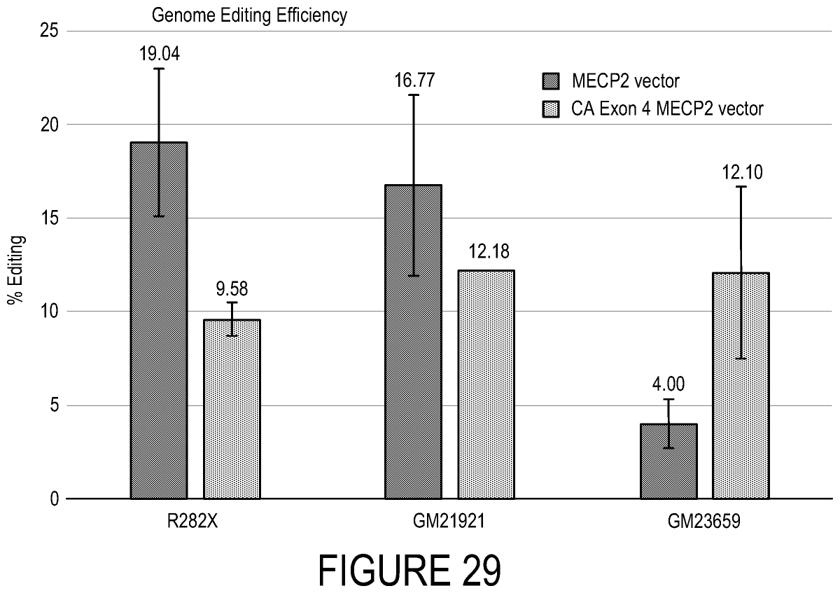

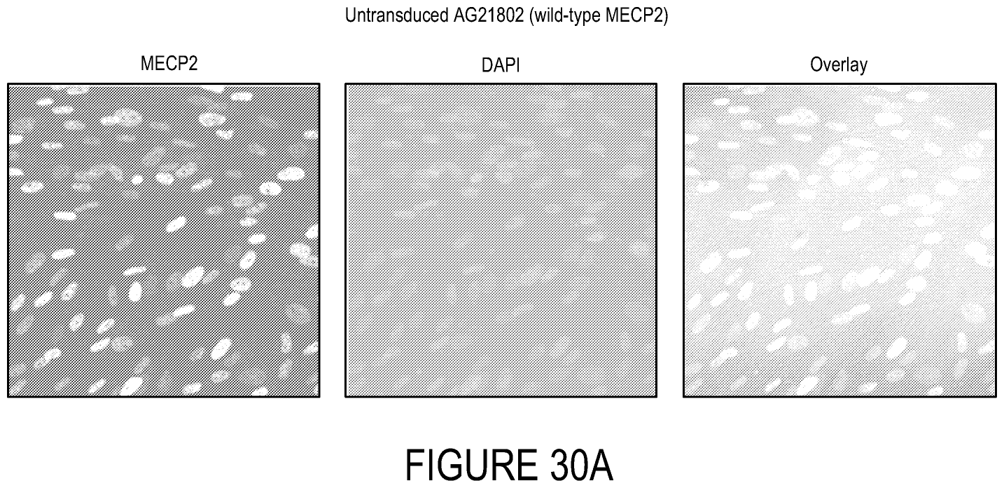

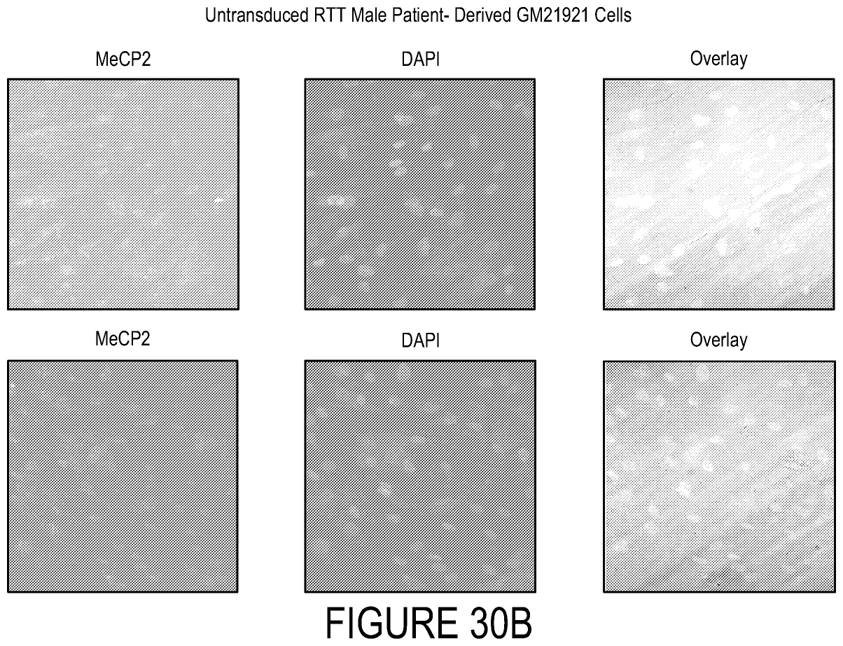

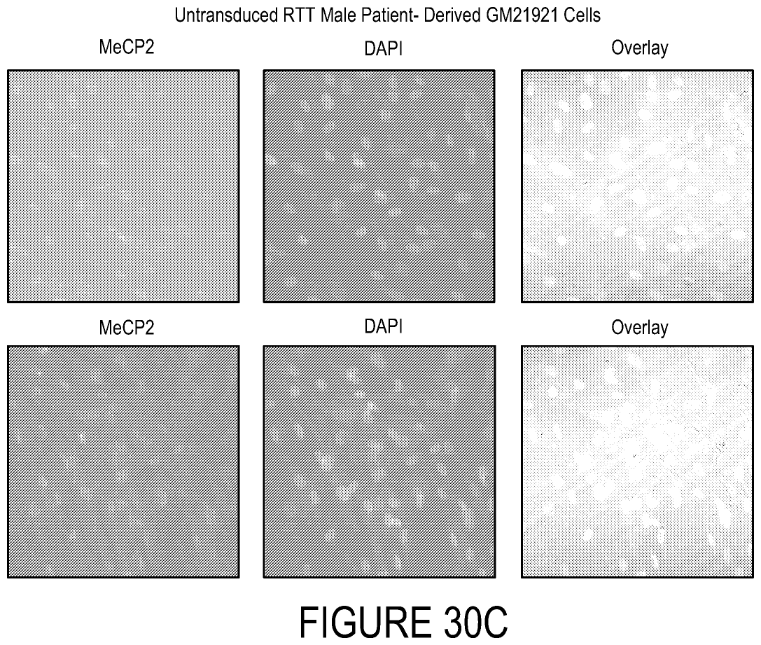

Editing Patterns Observed from 3′ Sequence Analysis of AAVHSC7-Edited R282X Patient-Derived Cells (FIG. 28)

(A) Recombination Pattern A sequence
(SEQ ID NO: 41)
CTAGTGACTACAAAGACCATGACGGTATCGATCGTACCTGAGTTCAAACTTGGGAAT
GTTCTAGATGGTGACTCAGGCCCAGGCACCAACCAGCAGAATGGGCCTCAGCCTGA
CAACCCTTCTGTACCAGGCCTGACTCTTTGGTTGCTGAACTTTGGAGAGGCCTGGGG
GGGTCAGCGGCAGGCAGACGAGTGAGTGGCTTTGGTGACAGGTCCTCAGGGGCAGC
CAGGCAGTGTGACTCTCGTTCAATAGTAACGTTTGTCAGAGCGTTGTCACCACCATC
CGCTCTGCCCTATCTCTGACATTGCTATGGAGAGCCTCTAATTGTTCCTTGTGTCTTT
CTGTTTGTCCCCACAGTCCCCAGGGAAAAGCCTTTCGCTCTAAAGTGGAGTTGATTG
CGTACTTCGAAAAGGTAGGCGACACATCCCTGGACCCTAATGATTTTGACTTCACGG
TAACTGGGAGAGGGAGCCCCTCCCGGCGAGAGCAGAAACCACCTAAGAAGCCCAA
ATCTCCCAAAGCTCCAGGAACTGGCAGAGGCCGGGGACGCCCCAAAGGGAGCGGC
ACCACGAGACCCAAGGCGGCCACGTCAGAGGGTGTGCAGGTGAAAAGGGTCCTGG
AGAAAAGTCCTGGGAAGCTCCTTGTCAAGATGCCTTTTCAAACTTCGCCAGGGGGCA
AGGCTGAGGGGGGTGGGGCCACCACATCCACCCAGGTCATGGTGATCAAACGCCCC
GGCAGGAAGCGAAAAGCTGAGGCCGACCCTCAGGCCATTCCCAAGAAACGGGGCC
GAAAGCCGGGGAGTGTGGTGGCAGCCGCTGCCGCCGAGGCCAAAAAGAAAGCCGT
GAAGGAGTCTTCTATCCGATCTGTGCAGGAGACCGTACTCCCCATCAAGAAGCGCA
AGACCCGGGAGACGGTCAGCATCGAGGTCAAGGAAGTGGTGAAGCCCCTGCTGGTG
TCCACCCTCGGTGAGAAGAGCGGGAAAGGACTGAAGACCTGTAAGAGCCCTGGGCG
GAAAAGCAAGGAGAGCAGCCCCAAGGGGCGCAGCAGCAGCGCCTCCTCACCCCCC
AAGAAGGAGCACCACCACCATCACCACCACTCAGAGTCCCCAAAGGCCCCCGTGCC
ACTGCTCCCACCCCTGCCCCCACCTCCACCTGAGCCCGAGAGCTCCGAGGACCCCAC
CAGCCCCCCTGAGCCCCAGGACTTGAGCAGCAGTGTCTGCAAAGAGGAGAAGATGC
CCAGAGGAGGCTCACTGGAGAGCGACGGCTGCCCCAAGGAGCCAGCTAAGACTCAG
CCCGCGGTTGCCACCGCCGCCACGGCCGCAGAAAAGTACAAACACCGAGGGGAGG
GAGAGCGCAAAGACATTGTTTCATCCTCCATGCCAAGGCCAAACAGAGAGGAGCCT
GTGGACAGCCGGACGCCCGTGACCGAGAGAGTTAGCTGACTTTACACGGAGCGGAT
TGCAAAGCAAACCAACAAGAATAAAGGCAGCTGTTGTCTCTTCTCCTTATGGGTAGG
GCTCTGACAAAGCTTCCCGATTAACTGAAATAAAAAATATTTTTTTTTCTTTCAGTAA
ACTTAGAGTTTCGTGGCTTCAGGGTGGGAGTAGTTGGAGCATTGGGGATGTTTTTCT
TACCGACAAGCACAGTCAGGTTGAAGACCTAACCAGGGCCAGAAGTAGCTTTGCAC
TTTTCTAAACTAGGCTCCTTCAACAAGGCTTGCTGCAGATACTACTGACCAGACAAG
CTGTTGACCAGGCACCTCCCCTCCCGCCCAAACCTTTCCCCCATGTGGTCGTTAGAG
ACAGAGCGACAGAGCAGTTGAGAGGACACTCCCGTTTTCGGTGCCATCAGTGCCCC
GTCTACAGCTCCCCCAGCTCCCCCCACCTCCCCCACTCCCAACCACGTTGGGACAGG
GAGGTGTGAGGCAGGAGAGACAGTTGGATTCTTTAGAGAAGATGGATATGACCAGT
GGCTATGGCCTGTGCGATCCCACCCGTGGTGGCTCAAGTCTGGCCCCACACCAGCCC
CAATCCAAAACTGGCAAGGACGCTTCACAGGACAGGAAAGTGGCACCTGTCTGCTC
CAGCTCTGGCATGGCTAGGAGGGGGGAGTCCCTTGAACTACTGGGTGTAGACTGGC
CTGAACCACAGGAGAGGATGGCCCAGGGTGAGGTGGCATGGTCCATTCTCAAGGGA
CGTCCTCCAACGGGTGGCGCTAGAGGCCATGGAGGCAGTAGGACAAGGTGCAGGCA
GGCTGGCCTGGGGTCAGGCCGGGCAGAGCACAGCGGGGTGAGAGGGATTCCTAATC
ACTCAGAGCAGTCTGTGACTTAGTGGACAGGGGAGGGGGCAAAGGGGGAGGAGAA
GAAAATGTTCTTCCAGTTACTTTCCAATTCTCCTTTAGGGACAGCTTAGAATTATTTG
CACTATTGAGTCTTCATGTTCCCACTTCAAAACAAACAGATGCTCTGAGAGCAAACT
GGCTTGAATTGGTGACATTTAGTCCCTCAAGCCACCAGATGTGACAGTGTTGAGAAC
TACCTGGATTTGTATATATACCTGCGCTTGTTTTAAAGTGGGCTCAGCACATAGGGTT
CCCACGAAGCTCCGAAACTCTAAGTGTTTGCTGCAATTTTATAAGGACTTCCTGATT
GGTTTCTCTTCTCCCCTTCCATTTCTGCCTTTTGTTCATTTCATCCTTTCACTTCT
(B) Recombination Pattern B sequence
(SEQ ID NO: 42)
CTAGTGACTACAAAGACCATGACGGTATCGATCGTACCTGAGTTCAAACTTGGGAAT
GTTCTAGATGGTGACTCAGGCCCAGGCACCAACCAGCAGAATGGGCCTCAGCCTGA
CAACCCTTCTGTACCAGGCCTGACTCTTTGGTTGCTGAACTTTGGAGAGGCCTGGGG
GGGTCAGCGGCAGGCAGACGAGTGAGTGGCTTTGGTGACAGGTCCTCAGGGGCAGC
CAGGCAGTGTGACTCTCGTTCAATAGTAACGTTTGTCAGAGCGTTGTCACCACCATC
CGCTCTGCCCTATCTCTGACATTGCTATGGAGAGCCTCTAATTGTTCCTTGTGTCTTT
CTGTTTGTCCCCACAGTCCCCAGGGAAAAGCCTTTCGCTCTAAAGTGGAGTTGATTG
CGTACTTCGAAAAGGTAGGCGACACATCCCTGGACCCTAATGATTTTGACTTCACGG
TAACTGGGAGAGGGAGCCCCTCCCGGCGAGAGCAGAAACCACCTAAGAAGCCCAA
ATCTCCCAAAGCTCCAGGAACTGGCAGAGGCCGGGGACGCCCCAAAGGGAGCGGC
ACCACGAGACCCAAGGCGGCCACGTCAGAGGGTGTGCAGGTGAAAAGGGTCCTGG
AGAAAAGTCCTGGGAAGCTCCTTGTCAAGATGCCTTTTCAAACTTCGCCAGGGGGCA
AGGCTGAGGGGGGTGGGGCCACCACATCCACCCAGGTCATGGTGATCAAACGCCCC
GGCAGGAAGCGAAAAGCTGAGGCCGACCCTCAGGCCATTCCCAAGAAACGGGGCT
GAAAGCCGGGGAGTGTGGTGGCAGCCGCTGCCGCCGAGGCCAAAAAGAAAGCCGT
GAAGGAGTCTTCTATCCGATCTGTGCAGGAGACCGTACTCCCCATCAAGAAGCGCA
AGACCCGGGAGACGGTCAGCATCGAGGTCAAGGAAGTGGTGAAGCCCCTGCTGGTG
TCCACCCTCGGTGAGAAGAGCGGGAAAGGACTGAAGACCTGTAAGAGCCCTGGGCG
GAAAAGCAAGGAGAGCAGCCCCAAGGGGCGCAGCAGCAGCGCCTCCTCACCCCCC
AAGAAGGAGCACCACCACCATCACCACCACTCAGAGTCCCCAAAGGCCCCCGTGCC
ACTGCTCCCACCCCTGCCCCCACCTCCACCTGAGCCCGAGAGCTCCGAGGACCCCAC
CAGCCCCCCTGAGCCCCAGGACTTGAGCAGCAGTGTCTGCAAAGAGGAGAAGATGC
CCAGAGGAGGCTCACTGGAGAGCGACGGCTGCCCCAAGGAGCCAGCTAAGACTCAG
CCCGCGGTTGCCACCGCCGCCACGGCCGCAGAAAAGTACAAACACCGAGGGGAGG
GAGAGCGCAAAGACATTGTTTCATCCTCCATGCCAAGGCCAAACAGAGAGGAGCCT
GTGGACAGCCGGACGCCCGTGACCGAGAGAGTTAGCTGACTTTACACGGAGCGGAT
TGCAAAGCAAACCAACAAGAATAAAGGCAGCTGTTGTCTCTTCTCCTTATGGGTAGG
GCTCTGACAAAGCTTCCCGATTAACTGAAATAAAAAATATTTTTTTTTCTTTCAGTAA
ACTTAGAGTTTCGTGGCTTCAGGGTGGGAGTAGTTGGAGCATTGGGGATGTTTTTCT
TACCGACAAGCACAGTCAGGTTGAAGACCTAACCAGGGCCAGAAGTAGCTTTGCAC
TTTTCTAAACTAGGCTCCTTCAACAAGGCTTGCTGCAGATACTACTGACCAGACAAG
CTGTTGACCAGGCACCTCCCCTCCCGCCCAAACCTTTCCCCCATGTGGTCGTTAGAG
ACAGAGCGACAGAGCAGTTGAGAGGACACTCCCGTTTTCGGTGCCATCAGTGCCCC
GTCTACAGCTCCCCCAGCTCCCCCCACCTCCCCCACTCCCAACCACGTTGGGACAGG
GAGGTGTGAGGCAGGAGAGACAGTTGGATTCTTTAGAGAAGATGGATATGACCAGT
GGCTATGGCCTGTGCGATCCCACCCGTGGTGGCTCAAGTCTGGCCCCACACCAGCCC
CAATCCAAAACTGGCAAGGACGCTTCACAGGACAGGAAAGTGGCACCTGTCTGCTC
CAGCTCTGGCATGGCTAGGAGGGGGGAGTCCCTTGAACTACTGGGTGTAGACTGGC
CTGAACCACAGGAGAGGATGGCCCAGGGTGAGGTGGCATGGTCCATTCTCAAGGGA
CGTCCTCCAACGGGTGGCGCTAGAGGCCATGGAGGCAGTAGGACAAGGTGCAGGCA
GGCTGGCCTGGGGTCAGGCCGGGCAGAGCACAGCGGGGTGAGAGGGATTCCTAATC
ACTCAGAGCAGTCTGTGACTTAGTGGACAGGGGAGGGGGCAAAGGGGGAGGAGAA
GAAAATGTTCTTCCAGTTACTTTCCAATTCTCCTTTAGGGACAGCTTAGAATTATTTG
CACTATTGAGTCTTCATGTTCCCACTTCAAAACAAACAGATGCTCTGAGAGCAAACT
GGCTTGAATTGGTGACATTTAGTCCCTCAAGCCACCAGATGTGACAGTGTTGAGAAC
TACCTGGATTTGTATATATACCTGCGCTTGTTTTAAAGTGGGCTCAGCACATAGGGTT
CCCACGAAGCTCCGAAACTCTAAGTGTTTGCTGCAATTTTATAAGGACTTCCTGATT
GGTTTCTCTTCTCCCCTTCCATTTCTGCCTTTTGTTCATTTCATCCTTTCACTTCT

### Example 6: Materials and Methods

### Cell Lines and Primary Cells

Primary fibroblasts and B-lymphoblastoid cell lines (B-LCLs) derived from Rett patients bearing different mutations in the MECP2 gene were obtained from Coriell Institute. Fibroblasts with MeCP2 R282X mutation were obtained from Dr. Narayanan, Tgen. Primary fibroblasts were passaged using 0.25% Trypsin-EDTA (Gibco, Ref 25200-056) and cultured in DMEM+GlutaMAX (Gibco, Ref 10569-010)+10% HI-FBS, and B-LCLs were cultured in RPMI Medium 1640 (Gibco, Ref 21870-076)+Glutamine+15% HI-FBS.

### Design of AAVHSC Editing Vector for Correction of MECP2

The AAVHSC editing vectors were designed to encode the MECP2 editing sequences flanked by AAV2 inverted repeats (ITR)s. The editing cassette comprised the following components: 1) 522 bp of intron 2, 2) exon 3, 3) intron 3, 4) exon 4 and 5) 797 bp of 3′ untranslated region (3′ UTR) sequence of the human MECP2 gene (NCBI genome assembly version GRCh38.p14), 6) a unique linker sequence of 28 bp was inserted in intron 2, 7) a 33 bp linker 2 sequence was inserted in intron 3, to allow detection of successful editing, 8) promoterless expression cassette consisting of a T2A sequence, followed by the Venus open reading frame (ORF) was inserted immediately downstream of exon 4 and before the start of the 3′ UTR. The linker sequence 1 in intron 2 and the linker sequence 2 in intron 3 were inserted to allow detection of edited genomes. The promoterless reporter expression cassette was designed to allow expression after correct insertion downstream from the MECP2 promoter. The reporter cassette is only for preclinical studies and will be removed from the final therapeutic vector.

### Packaging, Purification and Titration of AAVHSC Editing Vectors

The AAVHSC editing vectors were packaged in AAVHSC15 and AAVHSC7 capsids in HEK293 cells using HSV-1 (Herpes Simplex Virus) helper virus as described previously (Chatterjee et al., 1992; Fisher-Adams et al., 1996; Smith et al., 2018). Briefly, the packaged cells were harvested 72 h post-transfection and lysed by freeze-thaw and sonication. The cell lysates were then treated with Benzonase to degrade cellular genomic DNA and residual plasmid DNA. The lysate was further treated with sodium deoxycholate and trypsin prior to purification by two rounds of CsCl₂ density gradient centrifugation. Viral vector fractions were collected after the gradient centrifugation and dialyzed.

The vectors were titered by qPCR using primers and a probe specific for the Venus sequence. Titers were calculated from a standard curve. The sequences of primers and the probe used are:

qVenus-Fwd:
(SEQ ID NO: 25)
CTGCTGCCCGACAACCA
qVenus-Rev:
(SEQ ID NO: 26)
TGTGATCGCGCTTCTCGTT
qVenus probe:
(SEQ ID NO: 27)
FAM-AGCTACCAGTCCGCCCTGAGCAAAGA-TAMRA

### Transductions and Flow Cytometry Analysis

Cells were plated in a 96-well plate (30,000 cells per well) and transduced with the AAVHSC vectors at a particular multiplicity of infection (MOI) ranging from 150,000 to 450,000. After 48 h, the cells were harvested and pelleted, washed with 2% FBS in PBS twice, and resuspended. DAPI was added to the cells at a final concentration of 3 μM along with 200 μl PBS immediately before flow cytometric analysis. The flow cytometry was performed on 10,000 or 20,000 cells per sample using an Attune Nxt Flow Cytometer. The cells were first gated based on forward and side scatter. This was followed by gating on live cells by excluding DAPI-positive dead cells. The reporter gene expression was determined from the live cell population. The data was analyzed using FlowJo software.

### Transductions and Targeted Integration (TI) Analysis

Cells were plated in wells of a 24-well plate (200,000 cells per well) and transduced with the vector at indicated MOT. After 48 h, the cells were harvested, pelleted, washed and the pellets were frozen at −80° C. until further use.

Cells were thawed and genomic DNA was extracted from the cell pellet using standard protocols (Sambrook, 1989). TI assays were performed by amplification with a chromosome-specific primer, one of which annealed to the genome external to the homology arms and a primer specific for linker 2 sequence. Touchdown amplification was performed using either Q5 Hi Fidelity DNA polymerase or KAPA HiFi HotStart DNA polymerase. The PCR product was gel purified and either sequenced directly or cloned using NEB PCR cloning kit (Cat no. E1202). The clones were screened and clones that yielded the expected sized bands were sequenced by Sanger sequencing.

The primers used for TI assays are listed in the table below:

| Primer | Sequence | Description |
|---|---|---|
| MeCP2-USD-F2 | GCTCACAGTAAGGATGCCTAGATG | Forward primer |
| | (SEQ ID NO: 28) | upstream of the |
| | | 5′ homology arm |
| MeCP2-USD-F3 | GGGTTACCTGTTGTCAAGATAAGGAAAC | Forward primer |
| | (SEQ ID NO: 29) | upstream of the |
| | | 5′ homology arm |
| MeCP2-DSD-R2 | GGGAACATGAAGACTCAATAGTGC | Reverse primer |
| | (SEQ ID NO: 30) | downstream of 3′ |
| | | homology arm |
| MeCP2-DSD-R7 | AGAAGTGAAAGGATGAAATGAACAAAAG | Reverse primer |
| | (SEQ ID NO: 31) | downstream of 3′ |
| | | homology arm |
| 226-linker2-R | CCGTCATGGTCTTTGTAGTCACTAG | Reverse primer |
| | (SEQ ID NO: 32) | annealing to |
| | | linker 2 sequence |
| 226-linker2-F | CTAGTGACTACAAAGACCATGACGG | Forward primer |
| | (SEQ ID NO: 33) | annealing to |
| | | linker 2 sequence |
| 226-linker2-F1 | CAAAGACCATGACGGTATCGATC | Forward primer |
| | (SEQ ID NO: 34) | annealing to |
| | | linker 2 sequence |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

### EMBODIMENTS

Embodiment 1: A replication-defective adeno-associated virus (AAV) comprising: (a) an AAV capsid; and (b) a correction genome comprising: (i) an editing element for editing a target locus in the MeCP2 gene; (ii) a 5′ homology arm nucleotide sequence 5′ to the editing element having homology to a first genomic region 5′ to the target locus; and (iii) a 3′ homology arm nucleotide sequence 3′ to the editing element having homology to a second genomic region 3′ to the target locus; wherein the editing element comprises a MECP2 Exon 3 nucleic acid sequence that encodes a mutation-deficient MeCP2 Exon 3 protein sequence or portion thereof, or wherein the editing element comprises a MeCP2 Exon 4 nucleic acid sequence that encodes a mutation-deficient MeCP2 Exon 4 protein sequence.

Embodiment 2: The AAV of Embodiment 1, wherein the Exon 3 nucleic acid sequence encodes a wild-type MeCP2 Exon 3 protein sequence or a fragment thereof.

Embodiment 3: The AAV of Embodiment 2, wherein the Exon 3 nucleic acid sequence is a codon-altered nucleic acid sequence.

Embodiment 4: The AAV of Embodiment 1, wherein the Exon 4 nucleic acid sequence encodes a wild-type MeCP2 Exon 4 protein sequence or a fragment thereof.

Embodiment 5: The AAV of Embodiment 4, wherein the Exon 4 nucleic acid sequence is a codon-altered Exon 4 nucleic acid sequence.

Embodiment 6: The AAV of any one of Embodiments 1-3, wherein the editing element comprises said MeCP2 Exon 3 nucleic acid sequence or a portion thereof.

Embodiment 7: The AAV of Embodiment 6, wherein the editing element comprises SEQ ID NO: 1.

Embodiment 8: The AAV of Embodiments 6 or 7, wherein the 5′ homology arm nucleotide sequence comprises a MECP2 intron 2 nucleic acid sequence.

Embodiment 9: The AAV of Embodiment 8, wherein the 5′ homology arm nucleotide sequence comprises SEQ ID NO: 2.

Embodiment 10: The AAV of any one of Embodiments 6-9, wherein the 3′ homology arm nucleotide sequence comprises a MECP2 intron 3 nucleic acid sequence and exon 4 nucleic acid sequence.

Embodiment 11: The AAV of Embodiment 10, wherein the 3′ homology arm nucleotide sequence comprises SEQ ID NO: 3.

Embodiment 12: The AAV of any one of Embodiments 1 and 4-5, wherein the editing element comprises said MECP2 Exon 4 nucleic acid sequence.

Embodiment 13: The AAV of claim 12, wherein the editing element comprises SEQ ID NO: 4.

Embodiment 14: The AAV of Embodiment 13, wherein the 5′ homology arm nucleotide sequence comprises a MECP2 intron 2, exon 3 and intron 3 nucleic acid sequence.

Embodiment 15: The AAV of Embodiment 14, wherein the 5′ homology arm nucleotide sequence comprises SEQ ID NO: 5.

Embodiment 16: The AAV of any one of Embodiments 12-15, wherein the 3′ homology arm nucleotide sequence comprises a MECP2 3′ UTR nucleic acid sequence.

Embodiment 17: The AAV of Embodiment 16, wherein the 3′ homology arm nucleotide sequence comprises SEQ ID NO: 6.

Embodiment 18: The AAV of any one of Embodiments 1-17, wherein the AAV capsid comprises an AAV Clade F capsid protein.

Embodiment 19: The AAV of any one of Embodiments 1-18, wherein the codon-altered nucleic acid sequence is a nucleic acid sequence which is at least 80% homologous to SEQ ID NO: 7 or SEQ ID NO: 9.

Embodiment 20: The AAV of any one of Embodiments 1-19, wherein the editing element comprises a MECP2 Exon 3 nucleic acid sequence that encodes a mutation-deficient MeCP2 Exon 3 protein sequence or portion thereof and a MeCP2 Exon 4 nucleic acid sequence that encodes a mutation-deficient MeCP2 Exon 4 protein sequence.

Embodiment 21: A replication-defective adeno-associated virus (AAV) comprising a nucleic acid molecule comprising an editing element for editing a target locus in the MeCP2 gene, said nucleic acid molecule comprising a sequence which is at least 90% identical to SEQ ID NO: 13; wherein the editing element comprises a MECP2 Exon 3 nucleic acid sequence that encodes a mutation-deficient MeCP2 Exon 3 protein sequence or portion thereof; or wherein the editing element comprises a MeCP2 Exon 4 nucleic acid sequence that encodes a mutation-deficient MeCP2 Exon 4 protein sequence.

Embodiment 22: A replication-defective adeno-associated virus (AAV) comprising a nucleic acid molecule comprising an editing element for editing a target locus in the MeCP2 gene, said nucleic acid molecule comprising a sequence which is at least 90% identical to SEQ ID NO: 14; wherein the editing element comprises a MECP2 Exon 3 nucleic acid sequence that encodes a mutation-deficient MeCP2 Exon 3 protein sequence or portion thereof, or wherein the editing element comprises a MeCP2 Exon 4 nucleic acid sequence that encodes a mutation-deficient MeCP2 Exon 4 protein sequence.

Embodiment 23: A replication-defective adeno-associated virus (AAV) comprising a nucleic acid molecule comprising an editing element for editing a target locus in the MeCP2 gene, said nucleic acid molecule comprising a sequence which is at least 90% identical to SEQ ID NO: 15; wherein the editing element comprises a MECP2 Exon 3 nucleic acid sequence that encodes a mutation-deficient MeCP2 Exon 3 protein sequence or portion thereof, or wherein the editing element comprises a MeCP2 Exon 4 nucleic acid sequence that encodes a mutation-deficient MeCP2 Exon 4 protein sequence.

Embodiment 24: A replication-defective adeno-associated virus (AAV) comprising a nucleic acid molecule comprising an editing element for editing a target locus in the MeCP2 gene, said nucleic acid molecule comprising a sequence which is at least 90% identical to SEQ ID NO: 16; wherein the editing element comprises a MECP2 Exon 3 nucleic acid sequence that encodes a mutation-deficient MeCP2 Exon 3 protein sequence or portion thereof, or wherein the editing element comprises a MeCP2 Exon 4 nucleic acid sequence that encodes a mutation-deficient MeCP2 Exon 4 protein sequence.

Embodiment 25: A replication-defective adeno-associated virus (AAV) comprising a nucleic acid molecule comprising an editing element for editing a target locus in the MeCP2 gene, said nucleic acid molecule comprising a sequence which is at least 90% identical to SEQ ID NO: 17; wherein the editing element comprises a MECP2 Exon 3 nucleic acid sequence that encodes a mutation-deficient MeCP2 Exon 3 protein sequence or portion thereof, or wherein the editing element comprises a MeCP2 Exon 4 nucleic acid sequence that encodes a mutation-deficient MeCP2 Exon 4 protein sequence.

Embodiment 26: A method for correcting a mutation in a MECP2 gene in a cell, the method comprising transducing the cell with the replication-defective adeno-associated virus (AAV) of any one of Embodiments 1-25; wherein the cell is transduced without co-transducing or co-administering an exogenous nuclease or a nucleotide sequence that encodes an exogenous nuclease.

Embodiment 27: The method of Embodiment 26, wherein the cell is a hepatocyte, an endothelial cell, a neuronal cell, a glial cell, an astrocyte, a fibroblast, a P-lymphoblast, a dermal cell, an osteocyte, a mucosal cell, a myocyte, a blood cell, an adipocyte, or a connective tissue cell.

Embodiment 28: The method of Embodiment 26 or Embodiment 27, wherein the cell is in a mammalian subject and the AAV is administered to the subject in an amount effective to transduce the cell in the subject.

Embodiment 29: The method of any one of Embodiments 26-28, wherein MeCP2 expression level in the subject is preserved.

Embodiment 30: A method of treating a subject having a disease or disorder associated with a MeCP2 gene mutation, the method comprising administering to the subject an effective amount of the replication-defective adeno-associated virus (AAV) of any one of Embodiments 1-24; wherein the AAV is administered to the subject without an exogenous nuclease or a nucleotide sequence that encodes an exogenous nuclease.

Embodiment 31: The method of Embodiment 30, wherein the disease or disorder is Rett syndrome.

Embodiment 32: The method of Embodiment 30 or Embodiment 31, wherein the subject is a human subject.

Embodiment 33: The method of any one of Embodiments 30-32, wherein MeCP2 expression level in the subject is preserved.

### REFERENCES

1. Chatterjee, S., Johnson, P. R., and Wong, K. K., Jr. (1992). Dual-target inhibition of HIV-1 in vitro by means of an adeno-associated virus antisense vector. Science 258, 1485-1488.
2. Fisher-Adams, G., Wong, K. K., Jr., Podsakoff, G., Forman, S. J., and Chatterjee, S. (1996). Integration of adeno-associated virus vectors in CD34+ human hematopoietic progenitor cells after transduction. Blood 88, 492-504.
3. Sambrook, J. (1989). Molecular Cloning: A laboratory manual (Cold Spring Harbor Laboratory Press).
4. Smith, L. J., Wright, J., Clark, G., Ul-Hasan, T., Jin, X., Fong, A., Chandra, M., St Martin, T., Rubin, H., Knowlton, D., et al. (2018). Stem cell-derived clade F AAVs mediate high-efficiency homologous recombination-based genome editing. Proc Natl Acad Sci USA 115, E7379-E7388.

1-33. (canceled)

34. A replication-defective adeno-associated virus comprising:
(a) an replication-defective adeno-associated virus capsid; and
(b) a correction genome comprising:
(i) an editing element for editing a target locus in the MECP2 gene;
(ii) a 5′ homology arm nucleotide sequence 5′ to the editing element having homology to a first genomic region 5′ to the target locus; and
(iii) a 3′ homology arm nucleotide sequence 3′ to the editing element having homology to a second genomic region 3′ to the target locus;
wherein the editing element comprises a MECP2 Exon 3 nucleic acid sequence that encodes a mutation-deficient MECP2 Exon 3 protein sequence or portion thereof, or wherein the editing element comprises a MECP2 Exon 4 nucleic acid sequence that encodes a mutation-deficient MECP2 Exon 4 protein sequence.

35. The replication-defective adeno-associated virus of claim 34, wherein the Exon 3 nucleic acid sequence encodes: (i) a wild-type MECP2 Exon 3 protein sequence or a fragment thereof, or (ii) a wild-type MECP2 Exon 4 protein sequence or a fragment thereof.

36. The replication-defective adeno-associated virus of claim 35, wherein: (i) the Exon 3 nucleic acid sequence is a codon-altered nucleic acid sequence, and (ii) the Exon 4 nucleic acid sequence is a codon-altered Exon 4 nucleic acid sequence.

37. The replication-defective adeno-associated virus of claim 34, wherein the editing element comprises the MECP2 Exon 3 nucleic acid sequence or a portion thereof.

38. The replication-defective adeno-associated virus of claim 34, wherein the editing element comprises SEQ ID NO: 1.

39. The replication-defective adeno-associated virus of claim 34, wherein the 5′ homology arm nucleotide sequence comprises a MECP2 intron 2 nucleic acid sequence.

40. The replication-defective adeno-associated virus of claim 34, wherein the 5′ homology arm nucleotide sequence comprises SEQ ID NO:2, and the 3′ homology arm nucleotide sequence comprises SEQ ID NO:3.

41. The replication-defective adeno-associated virus of claim 34, wherein the 3′ homology arm nucleotide sequence comprises a MECP2 intron 3 nucleic acid sequence and exon 4 nucleic acid sequence.

42. The replication-defective adeno-associated virus of claim 34, wherein the editing element comprises the MECP2 Exon 4 nucleic acid sequence.

43. The replication-defective adeno-associated virus of claim 34, wherein the editing element comprises SEQ ID NO:4.

44. The replication-defective adeno-associated virus of claim 34, wherein the 5′ homology arm nucleotide sequence comprises a MECP2 intron 2, exon 3 and intron 3 nucleic acid sequence.

45. The replication-defective adeno-associated virus of claim 34, wherein the 3′ homology arm nucleotide sequence comprises a MECP2 3′ UTR nucleic acid sequence.

46. The replication-defective adeno-associated virus of claim 34, wherein the 5′ homology arm nucleotide sequence comprises SEQ ID NO:5, and the 3′ homology arm nucleotide sequence comprises SEQ ID NO:6.

47. The replication-defective adeno-associated virus of claim 34, wherein the replication-defective adeno-associated virus capsid comprises an replication-defective adeno-associated virus Clade F capsid protein.

48. The replication-defective adeno-associated virus of claim 34, wherein the codon-altered nucleic acid sequence is at least 80% homologous to SEQ ID NO:7 or SEQ ID NO:9.

49. The replication-defective adeno-associated virus of claim 34, wherein the editing element comprises a MECP2 Exon 3 nucleic acid sequence that encodes a mutation-deficient MECP2 Exon 3 protein sequence or portion thereof and a MECP2 Exon 4 nucleic acid sequence that encodes a mutation-deficient MECP2 Exon 4 protein sequence.

50. A replication-defective adeno-associated virus comprising a nucleic acid molecule comprising an editing element for editing a target locus in the MECP2 gene, wherein
(a) the nucleic acid molecule comprises a sequence which is at least 90% identical to SEQ ID NO:13, at least 90% identical to SEQ ID NO:14, at least 90% identical to SEQ ID NO:15, at least 90% identical to SEQ ID NO:16, at least 90% identical to SEQ ID NO:17;
(b) the editing element comprises:
(i) a MECP2 Exon 3 nucleic acid sequence that encodes a mutation-deficient MECP2 Exon 3 protein sequence or portion thereof, or
(ii) a MECP2 Exon 4 nucleic acid sequence that encodes a mutation-deficient MECP2 Exon 4 protein sequence.

51. A method for correcting a mutation in a MECP2 gene in a cell, the method comprising transducing the cell with the replication-defective adeno-associated virus of claim 34; wherein the cell is transduced without co-transducing or co-administering an exogenous nuclease or a nucleotide sequence that encodes an exogenous nuclease.

52. The method of claim 51, wherein the cell is a hepatocyte, an endothelial cell, a neuronal cell, a glial cell, an astrocyte, a fibroblast, a P-lymphoblast, a dermal cell, an osteocyte, a mucosal cell, a myocyte, a blood cell, an adipocyte, or a connective tissue cell.

53. A method of treating a subject having a disease or disorder associated with a MECP2 gene mutation, the method comprising administering to the subject an effective amount of the replication-defective adeno-associated virus of claim 34; wherein the replication-defective adeno-associated virus is administered to the subject without an exogenous nuclease or a nucleotide sequence that encodes an exogenous nuclease.


SEQUENCE LISTING (42 sequences)

SEQ ID NO: 1 - DNA, 348 bp, synthetic construct
  source 1..348: mol_type = other DNA
gaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatc

SEQ ID NO: 2 - DNA, 2001 bp, synthetic construct
  source 1..2001: mol_type = other DNA
gactcagagacatgaaaaggaagctctggctgggcacggtggctcacgcctgtaatcccagcactttgggaggctgaggcgggtcgatcacctgaggtcaggagttcgagaccagcctgcccaacatggtgaaaccccgtctccactaaacatacaaaaattagcctggcgtggtagcacacgcctgtgatcccagctactccggaggctgaggcaggagaataacttgaacccggcaggcggaggttgcgatgagccgatattatgccatcggcaacagagcgagactccatctcaaaaaaaaaacaaaaaacaaaacaaggcgagctctgtgctgggacagattagggacccctctttacagcaagaaagactgctctgtgggttgtaggatgcctttgtgtatgcagtggctctaggtgactctggcagccacactctgggccctaaacttctggaggaagatacaggatagggaggaactcaggggtgagtcatggtggggacaagacattccctcactctaagaccttgtcactagattggaacatctcttgcctccctacacctgacctgatggttctggagagatacgtccttgcagcttctgagtcccagcactaagcagcctttggtaacttccctacatcatttgagttctggtttcctaaggatgcttgccagtgagtgccatggtgccctcattgcacagtctgtgcagtgtagacaagaggggaagtctcttggggtagaccagccgcaaggcggtgactagcactgatgtgaaccacatgggacaggggagttgtggggctgagaacacggagggtgggagtagtcatgctcttttccagaatgaactgctaacgaagggactcgcaggtggctgctgcttctttccaagctgcccctgttgttgcagaggctctggagtcctaggaggtttcacggtggcatactcgacagagtactagagcatcatggccagatagtgatgctggggtgtggggcctcacggtggccatttctgacgagaccccaccgggccaaagtgatgtgtagagaaggagctgcttcggtcaccagaaaagaacggggaagcctcccacactagaataggtagggtgctcttctccaccggcaggaaggggatgtatggctctgcctggaccacacctttttccttgctcttccctccgcatctgctgtggccgaggccattcctcatcagggaacatgtgttagaggctcacgccacctgggaccacttgtcttatcacccccaggaccctaggcggtagtttcctgtggcctgagttagctgatatttatatagtgccattgtgccttttctcctgtgatgctcacagtaaggatgcctagatggggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagaccccttccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctggggaaggacatcaagatctgagtgtatgatggcctggggccttgcatgtggtgggggtccaagcctgcctctgctcacttgttctgcagactggcatgttctctgtgatacttacatacttgtttaacacttcagg

SEQ ID NO: 3 - DNA, 1839 bp, synthetic construct
  source 1..1839: mol_type = other DNA
aagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcaccgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagc

SEQ ID NO: 4 - DNA, 1080 bp, synthetic construct
  source 1..1080: mol_type = other DNA
ccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagc

SEQ ID NO: 5 - DNA, 1629 bp, synthetic construct
  source 1..1629: mol_type = other DNA
caggttggctggctccaaacctgtgtgtgctttccatgacactgtactgtgtgcctcattgagcctcattctagaaaaccaaaaacacacccaaggcccggccttcacaaaggagaccccttccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctggggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcaccgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagt

SEQ ID NO: 6 - DNA, 2003 bp, synthetic construct
  source 1..2003: mol_type = other DNA
tgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttca

SEQ ID NO: 7 - DNA, 351 bp, synthetic construct
  source 1..351: mol_type = other DNA
ggaggagaaaagtgaggatcaggatctgcaaggtttgaaagataagccactgaaattcaagaaagtgaaaaaggacaaaaaggaggaaaaggaaggcaaacacgaaccggtgcagcccagtgcacatcattccgccgaacctgctgaagctggcaaggctgaaaccagtgagggcagtggttctgcacccgccgtgcccgaggcctccgcatctcctaagcagcgccggtctatcatccgagataggggcccgatgtatgacgatccaacactccccgagggctggacccgcaaattgaaacagcggaagtccggacggtccgccggcaaatatgacgtgtatcttatcaa

SEQ ID NO: 8 - protein, 116 aa, synthetic construct
EEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLI

SEQ ID NO: 9 - DNA, 1084 bp, synthetic construct
  source 1..1084: mol_type = other DNA
tccccaggggaaggccttcaggtccaaggtcgagctgatcgcctattttgagaaagtgggagacactagcctcgaccccaatgacttcgactttaccgtgaccggccggggaagtccaagcagacgagagcagaagccccccaaaaaacctaagtccccaaaggcccccgggaccggacggggtagagggaggcctaagggctctggaacgacccggccaaaagccgcaaccagtgaaggcgttcaggtcaagcgcgtgcttgagaagtcacccggaaaactgctggtgaaaatgcccttccagacctcgcccggaggaaaagccgagggcggcggagccacgactagcacgcaagtgatggtaattaagaggccaggacgcaaacgaaaggccgaggctgacccccaggcgatcccgaaaaagagaggtcgaaaacccggctcagtggtcgccgcagccgcggctgaggccaagaaaaaggctgttaaagagtcctccattcgatccgtacaggagacagtgctgccaattaaaaaaaggaaaacgagagaaaccgtgagtattgaggtgaaagaggtcgtgaaacctctcctggtcagcactctgggcgagaaatctggaaaggggctcaaaacgtgcaaaagtcccggcagaaagtctaaagagagttccccaaaaggaaggagctcctccgccagcagtcctcctaaaaaagagcaccatcaccaccaccatcacagtgagagccccaaagcacctgttccccttctgccccctctcccaccccccccccccgaacctgagtctagcgaggacccaacgtctcctcccgagcctcaggacctgtcttcttctgtgtgtaaggaggagaaaatgccacgggggggaagtttggagtctgacggttgtccaaaagagcccgccaaaacccagcctgccgtggcgactgctgccaccgcagccgagaaatataagcaccgaggcgagggggagaggaaggacatcgtgagtagcagcatgccccgccccaaccgggaggagcccgtcgattctagaaccccagtaacagagcgggtgtcttga

SEQ ID NO: 10 - protein, 360 aa, synthetic construct
PQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 11 - DNA, 100 bp, synthetic construct
  source 1..100: mol_type = other DNA
ggggccttgcatgtggtgggggtccaagcctgcctctgctcacttgttctgcagactggcatgttctctgtgatacttacatacttgtttaacacttcag

SEQ ID NO: 12 - DNA, 100 bp, synthetic construct
  source 1..100: mol_type = other DNA
ctttacacggagcggattgcaaagcaaaccaacaagaataaaggcagctgttgtctcttctccttatgggtagggctctgacaaagcttcccgattaact

SEQ ID NO: 13 - DNA, 4702 bp, synthetic construct
  source 1..4702: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggcggccgcatatggaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaagtaagtaagagcaagtcttgtgtctatagaacaagaaggccaggtgggtaggaaggctaggatggaaaagctttattgcactgaaatggcttcccaaaaaggctggacaccatgccttggttaaaatggaggaatgttttggtggtggattaggtctgggtaccaaccaaagatattcagtctagcccaatgacccccaagtactagacctcactccttcagtttaaggattgtggaaaagccagggagtacagaggagtgggcttgaaggccggcaggtcctctgttttcagatccagctgttttggcttctgagcaagctgtaacatggtttctacccatgcaactagtgactacaaagaccatgacggtatcgattctggcccggttgtgttccagtttgttccttgttcacttacaatgtcttagaagaaagcttgtcagcatattttctatctcagtctctatgttgtcaaaaagcttctgactattgtttgtgtctctctgtttgtttctacagtccccaggggaaggcttttcggtctaaagtagagcttattgcatactttgagaaagtgggggataccagccttgacccaaatgactttgatttcacggtaactggaaggggaagccctagcagaagggagcagaagcctccaaaaaagcctaaatcccctaaggctcctgggactggcagaggtcgggggcggcctaaaggaagcggcactggaaggcctaaagcagcagcatcagagggtgttcaggtgaagagagtcctggaaaaaagcccaggaaagttggttgtcaagatgccattccaagcatcgccaggaggtaaaggtgaagggggtggagctaccacatccgctcaggtcatggtgattaaacggccaggcaggaagcgaaaggctgaggctgaccctcaggccattccaaaaaaacggggtaggaagccaggatcagtggtggctgcagcagctgcagaggccaaaaaaaaggccgtgaaagaatccagcatacggtccgtgcacgagactgtgctccctatcaaaaagcggaaaacccgggaaacggtcagcatcgaggtcaaggaggtggtgaaacctctgctggtgagcaccttgggtgaaaaaagcggaaaagggctgaaaacctgcaagagcccaggacgtaagagcaaagagagcagccctaagggacggagcagcagtgcctcttctcctccaaaaaaggaacatcaccaccatcatcaccattcagagtctacaaaagcccctatgcctctgctcccttctcctcctcctccagaaccagagagctccgaggatcctatcagccctccagaaccacaggaccttagcagcagcatctgcaaagaggagaaaatgcctcgagggggctcactggagagcgacggctgccctaaagagcctgctaagactcagccaatggtggctactaccactaccgtcgctgagaaatataaacatcgcggagaaggggaacggaaagatatcgtcagttcctctatgcctagacctaatagggaagaaccagtggattcccggactcctgtcactgaaagggtttccgctagcggcagcggagagggcagaggaagtcttctaacatgcggtgacgtggaggagaatcccggccctaggctcgagatggaggatgccaagaatattaagaaaggccctgccccattctaccctctggaagatggcactgctggtgagcaactgcacaaggccatgaagaggtatgccctggtccctggcaccattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttaggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacttaccccctggcttcaatgagtatgactttgtgcctgagagctttgacagggacaagaccattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcctacaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgacattgcctactgggatgaggatgagcactttttcattgtggacaggctgaagagcctcatcaagtacaaaggctaccaagtggcacctgctgagctagagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggtggcaaaatcgccgtctaggaattcctttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaaccagggccagaagtagctttgcacttttctaaactaggctccttcaacaaggcttgctgcagatactactgaccagacaagctgttgaccaggcactccccccaacaatatcctccctcttccccccccccacccccgccccgtgtgctcgttagggcaattgagaggacactcccatttttggtgccattgatgccctgtccataatagcttccctgacttttacaccaccccaactcccaatctgaaggactgggaggtgtgatgcaggagaaactatgggactcttgggagaagactatggagttggccagtgattaaggcccagtaattccaactgtggtagcacagatctggctccacatcaacccaatccaaaactgacaaggatattttgcaaaaaaagaaagtggcacctgtctgatccagctctgacatggctagaggtgagtcctaaactgatggcttataaactagcctgagccacagaagagtatggcccagagtgaagtgtcatcatctgttcacaaggcatgctcccctagaagataatgctaaagagcctgcaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 14 - DNA, 4700 bp, synthetic construct
  source 1..4700: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggcggccgcatatgcaggttggctggctccaaacctgtgtgtgctttccatgacactgtactgtgtgcctcattgagcctcattctagaaaaccaaaaacacacccaaggcccggccttcacaaaggagaccccttccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctgggaggtttcccggaaccttttggaaaaccggaaggacatcaagatctgagtgtatgatggcctggggccttgcatgtggtgggggtccaagcctgcctctgctcacttgttctgcagactggcatgttctctgtgatacttacatacttgtttaacacttcagggaagagaaaagtgaggatcaggatctgcaaggtttgaaagataagccactgaaattcaagaaagtgaaaaaggacaaaaaggaggaaaaggaaggcaaacacgaaccggtgcagcccagtgcacatcattccgccgaacctgctgaagctggcaaggctgaaaccagtgagggcagtggttctgcacccgccgtgcccgaggcctccgcatctcctaagcagcgccggtctatcatccgagataggggcccgatgtatgacgatccaacactccccgagggctggacccgcaaattgaaacagcggaagtccggacggtccgccggcaaatatgacgtgtatcttatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacggtatcgatcgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagcgcggccgccggcagcggagagggcagaggaagtcttctaacatgcggtgacgtggaggagaatcccggccctaggggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaactcgagtgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacatgcatttcctgcaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 15 - DNA, 4700 bp, synthetic construct
  source 1..4700: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggcggccgcatatgcaggttggctggctccaaacctgtgtgtgctttccatgacactgtactgtgtgcctcattgagcctcattctagaaaaccaaaaacacacccaaggcccggccttcacaaaggagaccccttccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctgggaggtttcccggaaccttttggaaaaccggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacggtatcgatcgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccaggggaaggccttcaggtccaaggtcgagctgatcgcctattttgagaaagtgggagacactagcctcgaccccaatgacttcgactttaccgtgaccggccggggaagtccaagcagacgagagcagaagccccccaaaaaacctaagtccccaaaggcccccgggaccggacggggtagagggaggcctaagggctctggaacgacccggccaaaagccgcaaccagtgaaggcgttcaggtcaagcgcgtgcttgagaagtcacccggaaaactgctggtgaaaatgcccttccagacctcgcccggaggaaaagccgagggcggcggagccacgactagcacgcaagtgatggtaattaagaggccaggacgcaaacgaaaggccgaggctgacccccaggcgatcccgaaaaagagaggtcgaaaacccggctcagtggtcgccgcagccgcggctgaggccaagaaaaaggctgttaaagagtcctccattcgatccgtacaggagacagtgctgccaattaaaaaaaggaaaacgagagaaaccgtgagtattgaggtgaaagaggtcgtgaaacctctcctggtcagcactctgggcgagaaatctggaaaggggctcaaaacgtgcaaaagtcccggcagaaagtctaaagagagttccccaaaaggaaggagctcctccgccagcagtcctcctaaaaaagagcaccatcaccaccaccatcacagtgagagccccaaagcacctgttccccttctgccccctctcccaccccccccccccgaacctgagtctagcgaggacccaacgtctcctcccgagcctcaggacctgtcttcttctgtgtgtaaggaggagaaaatgccacgggggggaagtttggagtctgacggttgtccaaaagagcccgccaaaacccagcctgccgtggcgactgctgccaccgcagccgagaaatataagcaccgaggcgagggggagaggaaggacatcgtgagtagcagcatgccccgccccaaccgggaggagcccgtcgattctagaaccccagtaacagagcgggtgtctgcggccgccggcagcggagagggcagaggaagtcttctaacatgcggtgacgtggaggagaatcccggccctaggggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaactcgagtgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacatgcatttcctgcaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 16 - DNA, 4285 bp, synthetic construct
  source 1..4285: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggcggccgcatatgccacactagaataggtagggtgctcttctccaccggcaggaaggggatgtatggctctgcctggaccacacctttttccttgctcttccctccgcatctgctgtggccgaggccattcctcatcagggaacatgtgttagaggctcacgccacctgggaccacttgtcttatcacccccaggaccctaggcggtagtttcctgtggcctgagttagctgatatttatatagtgccattgtgccttttctcctgtgatgctcacagtaaggatgcctagatggggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagaccccttccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctgggaggtttcccggaaccttttggaaaaccggaaggacatcaagatctgagtgtatgatggcctggggccttgcatgtggtgggggtccaagcctgcctctgctcacttgttctgcagactggcatgttctctgtgatacttacatacttgtttaacacttcagggaagagaaaagtgaggatcaggatctgcaaggtttgaaagataagccactgaaattcaagaaagtgaaaaaggacaaaaaggaggaaaaggaaggcaaacacgaaccggtgcagcccagtgcacatcattccgccgaacctgctgaagctggcaaggctgaaaccagtgagggcagtggttctgcacccgccgtgcccgaggcctccgcatctcctaagcagcgccggtctatcatccgagataggggcccgatgtatgacgatccaacactccccgagggctggacccgcaaattgaaacagcggaagtccggacggtccgccggcaaatatgacgtgtatcttatcaatccccaggggaaggccttcaggtccaaggtcgagctgatcgcctattttgagaaagtgggagacactagcctcgaccccaatgacttcgactttaccgtgaccggccggggaagtccaagcagacgagagcagaagccccccaaaaaacctaagtccccaaaggcccccgggaccggacggggtagagggaggcctaagggctctggaacgacccggccaaaagccgcaaccagtgaaggcgttcaggtcaagcgcgtgcttgagaagtcacccggaaaactgctggtgaaaatgcccttccagacctcgcccggaggaaaagccgagggcggcggagccacgactagcacgcaagtgatggtaattaagaggccaggacgcaaacgaaaggccgaggctgacccccaggcgatcccgaaaaagagaggtcgaaaacccggctcagtggtcgccgcagccgcggctgaggccaagaaaaaggctgttaaagagtcctccattcgatccgtacaggagacagtgctgccaattaaaaaaaggaaaacgagagaaaccgtgagtattgaggtgaaagaggtcgtgaaacctctcctggtcagcactctgggcgagaaatctggaaaggggctcaaaacgtgcaaaagtcccggcagaaagtctaaagagagttccccaaaaggaaggagctcctccgccagcagtcctcctaaaaaagagcaccatcaccaccaccatcacagtgagagccccaaagcacctgttccccttctgccccctctcccaccccccccccccgaacctgagtctagcgaggacccaacgtctcctcccgagcctcaggacctgtcttcttctgtgtgtaaggaggagaaaatgccacgggggggaagtttggagtctgacggttgtccaaaagagcccgccaaaacccagcctgccgtggcgactgctgccaccgcagccgagaaatataagcaccgaggcgagggggagaggaaggacatcgtgagtagcagcatgccccgccccaaccgggaggagcccgtcgattctagaaccccagtaacagagcgggtgtctgcggccgccggcagcggagagggcagaggaagtcttctaacatgcggtgacgtggaggagaatcccggccctaggggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaactcgagtgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacatgcatttcctgcaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 17 - DNA, 4701 bp, synthetic construct
  source 1..4701: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggcggccgcatatggaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaagtaagtaagagcaagtcttgtgtctatagaacaagaaggccaggtgggtaggaaggctaggatggaaaagctttattgcactgaaatggcttcccaaaaaggctggacaccatgccttggttaaaatggaggaatgttttggtggtggattaggtctgggtaccaaccaaagatattcagtctagcccaatgacccccaagtactagacctcactccttcagtttaaggattgtggaaaagccagggagtacagaggagtgggcttgaaggccggcaggtcctctgttttcagatccagctgttttggcttctgagcaagctgtaacatggtttctacccatgcaactagtgactacaaagaccatgacggtatcgattctggcccggttgtgttccagtttgttccttgttcacttacaatgtcttagaagaaagcttgtcagcatattttctatctcagtctctatgttgtcaaaaagcttctgactattgtttgtgtctctctgtttgtttctacagtccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagcgctagcggcagcggagagggcagaggaagtcttctaacatgcggtgacgtggaggagaatcccggccctaggctcgagatggaggatgccaagaatattaagaaaggccctgccccattctaccctctggaagatggcactgctggtgagcaactgcacaaggccatgaagaggtatgccctggtccctggcaccattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttaggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacttaccccctggcttcaatgagtatgactttgtgcctgagagctttgacagggacaagaccattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcctacaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgacattgcctactgggatgaggatgagcactttttcattgtggacaggctgaagagcctcatcaagtacaaaggctaccaagtggcacctgctgagctagagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggtggcaaaatcgccgtctaggaattcctttacatagagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcggggtgggagtagttggagcattgggatgtttttcttaccgacaagcacagtcaggttgaagacctaaccagggccagaagtagctttgcacttttctaaactaggctccttcaacaaggcttgctgcagatactactgaccagacaagctgttgaccaggcactccccccaacaatatcctccctcttccccccccccacccccgccccgtgtgctcgttagggcaattgagaggacactcccatttttggtgccattgatgccctgtccataatagcttccctgacttttacaccaccccaactcccaatctgaaggactgggaggtgtgatgcaggagaaactatgggactcttgggagaagactatggagttggccagtgattaaggcccagtaattccaactgtggtagcacagatctggctccacatcaacccaatccaaaactgacaaggatattttgcaaaaaaagaaagtggcacctgtctgatccagctctgacatggctagaggtgagtcctaaactgatggcttataaactagcctgagccacagaagagtatggcccagagtgaagtgtcatcatctgttcacaaggcatgctcccctagaagataatgctaaagagcctgcaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcca

SEQ ID NO: 18 - DNA, 1446 bp, synthetic construct
  source 1..1446: mol_type = other DNA
gctcacagtaaggatgcctagatggggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagacccctcccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctggggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacgg

SEQ ID NO: 19 - DNA, 1422 bp, synthetic construct
  source 1..1422: mol_type = other DNA
gggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagacccctcccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctggggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacatggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacgg

SEQ ID NO: 20 - DNA, 1474 bp, synthetic construct
  source 1..1474: mol_type = other DNA
gctcacagtaaggatgcctagatggggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagacccctcccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctgggaggtttcccggaaccttttggaaaaccggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacgg

SEQ ID NO: 21 - DNA, 1450 bp, synthetic construct
  source 1..1450: mol_type = other DNA
gggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagaccccttccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctgggaggtttcccggaaccttttggaaaaccggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacgg

SEQ ID NO: 22 - DNA, 2410 bp, synthetic construct
  source 1..2410: mol_type = other DNA
cgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttccc

SEQ ID NO: 23 - DNA, 2410 bp, synthetic construct
  source 1..2410: mol_type = other DNA
cgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccagggaaaagcctttcgctgtaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttccc

SEQ ID NO: 24 - DNA, 2432 bp, synthetic construct
  source 1..2432: mol_type = other DNA
caaagaccatgacggtatcgatcgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttccc

SEQ ID NO: 25 - DNA, 17 bp, synthetic construct
  source 1..17: mol_type = other DNA
ctgctgcccgacaacca

SEQ ID NO: 26 - DNA, 19 bp, synthetic construct
  source 1..19: mol_type = other DNA
tgtgatcgcgcttctcgtt

SEQ ID NO: 27 - DNA, 26 bp, synthetic construct
  source 1..26: mol_type = other DNA
agctaccagtccgccctgagcaaaga

SEQ ID NO: 28 - DNA, 24 bp, synthetic construct
  source 1..24: mol_type = other DNA
gctcacagtaaggatgcctagatg

SEQ ID NO: 29 - DNA, 28 bp, synthetic construct
  source 1..28: mol_type = other DNA
gggttacctgttgtcaagataaggaaac

SEQ ID NO: 30 - DNA, 24 bp, synthetic construct
  source 1..24: mol_type = other DNA
gggaacatgaagactcaatagtgc

SEQ ID NO: 31 - DNA, 28 bp, synthetic construct
  source 1..28: mol_type = other DNA
agaagtgaaaggatgaaatgaacaaaag

SEQ ID NO: 32 - DNA, 25 bp, synthetic construct
  source 1..25: mol_type = other DNA
ccgtcatggtctttgtagtcactag

SEQ ID NO: 33 - DNA, 25 bp, synthetic construct
  source 1..25: mol_type = other DNA
ctagtgactacaaagaccatgacgg

SEQ ID NO: 34 - DNA, 23 bp, synthetic construct
  source 1..23: mol_type = other DNA
caaagaccatgacggtatcgatc

SEQ ID NO: 35 - DNA, 1422 bp, synthetic construct
  source 1..1422: mol_type = other DNA
gggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagacccctcccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctggggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacgg

SEQ ID NO: 36 - DNA, 1450 bp, synthetic construct
  source 1..1450: mol_type = other DNA
gggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagacccctcccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctgggaggtttcccggaaccttttggaaaaccggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagccaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacgg

SEQ ID NO: 37 - DNA, 1450 bp, synthetic construct; identical to SEQ ID NO: 21
  source 1..1450: mol_type = other DNA

SEQ ID NO: 38 - DNA, 1450 bp, synthetic construct; identical to SEQ ID NO: 36
  source 1..1450: mol_type = other DNA

SEQ ID NO: 39 - DNA, 1422 bp, synthetic construct; identical to SEQ ID NO: 35
  source 1..1422: mol_type = other DNA

SEQ ID NO: 40 - DNA, 1422 bp, synthetic construct
  source 1..1422: mol_type = other DNA
gggttacctgttgtcaagataaggaaactgaagcacagaatgctgaggtcatttgctgggttcatgtttggaaagcggcaaaggatttcagtgcaggttggctggctccaaacctgtgtgtgctttccatgacactgtactgtgtgcctcattgagcctcattctagaaaaccaaaaacacacccaaggcccggccttcacaaaggagacccctcccccatttggctccctttccagcagtcgacggcctcttgtcagccatcgagcccagagtcccttgaagtgcgactcatgctggggtggtatgctcaggagccgcagtgtttccgctcagaggaaagggctctgattctcctgcagtgctaggagacttgtgggtggccacagtgcaggtcaggcacaccggccagcaccacccacagcccaaattcctaaagaaatatttgggtcccagcttggcccgagtctctgttgtcctggggaaggacatcaagatctgagtgtatgatggcctggggccttgcatgtggtgggggtccaagcctgcctctgctcacttgttctgcagactggcatgttctctgtgatacttacatacttgtttaacacttcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaagtaagtaagagcaactcctatctctacagggcagggagggcagggacaaggatccctcatggagcaggaaaatgtatgtgcccagggtggggtcggggggaacataaacaatgaacactgagaccaggtgtgcttgaaatgaccgtgtacagaggtcgctgccctgagtgggaagttctcaaggtagcaggccctctatcctctccacacctcaagtctttatctggggatggaatagctgcggaagcagaggaacttgcagagctaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcacactagtgactacaaagaccatgacgg

SEQ ID NO: 41 - DNA, 2704 bp, synthetic construct
  source 1..2704: mol_type = other DNA
ctagtgactacaaagaccatgacggtatcgatcgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagtgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttct

SEQ ID NO: 42 - DNA, 2704 bp, synthetic construct
  source 1..2704: mol_type = other DNA
ctagtgactacaaagaccatgacggtatcgatcgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggctgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagtgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttct